(19) United States
(12) Patent Application Publication
(10) Pub. No.: US 2023/0357824 A1
(43) Pub. Date: Nov. 9, 2023

(54) EXTRACTION SOLUTION FOR PREPARING A BIOLOGICAL SAMPLE FOR AMPLIFICATION BASED PATHOGEN DETECTION

(71) Applicant: Qiagen GmbH, Hilden (DE)

(72) Inventors: Miroslav VRANES, Hilden (DE); Norbert HOCHSTEIN, Hilden (DE); Ralf PEIST, Hilden (DE); Stefan CORNELIUS, Hilden (DE); Thorsten SINGER, Hilden (DE); Kerstin STEINERT, Hilden (DE); Kai TE KAAT, Hilden (DE)

(21) Appl. No.: 18/029,538

(22) PCT Filed: Oct. 6, 2021
(86) PCT No.: PCT/EP2021/077632

§ 371 Date: Mar. 30, 2023

Related U.S. Application Data
(60) Provisional application No. 63/088,423, filed on Oct. 6, 2020.

(30) Foreign Application Priority Data
Oct. 6, 2020 (EP) 20200425.5
Oct. 6, 2020 (EP) 20200426.3
Dec. 16, 2020 (EP) 20214412.7

(51) Int. Cl. C12Q 1/70; C12Q 1/6806

(52) U.S. Cl. CPC C12Q 1/6806; C12Q 1/701

(57) ABSTRACT

The present invention provides method and kits for amplification based detection of target nucleic acids and pathogens using crude biological samples without prior target purification.

### FIELD OF INVENTION

The invention describes an extraction composition which allows the direct detection of a pathogen by PCR, especially a RNA virus as SARS-CoV-2, by its nucleic acid genome without prior extraction and purification of the target nucleic acid.

The extraction composition according to the invention is added to a crude biological sample whereby the so prepared sample is made compatible for a subsequent direct amplification of the one or more target nucleic acids without prior purification of the target nucleic acids. The technology of the invention increases the sensitivity compared to prior art methods. Furthermore, means are provided that allow to avoid a loss of sensitivity due to inhibition of the amplification reaction due to components comprised in the prepared biological sample.

### BACKGROUND OF THE INVENTION

Methods for detecting the presence or absence of target nucleic acids in a biological sample are widely in use and of particular relevance in the diagnostic field. Such methods are widely used for determining whether a subject is infected with a pathogen of interest. A standard procedure for the detection of a pathogen, such as bacteria or virus, is the proof for the presence of the genomic nucleic acid of the pathogen, i.e. DNA or RNA. In standard prior art methods, the nucleic acid of the pathogen, such as a virus, is purified from a patient’s sample using a sample preparation method as described in the literature. The purified nucleic acid is then applied to a nucleic acid amplification reaction, amplified and detected for determining the presence or absence of the pathogen.

Due to its sensitivity, polymerase chain reaction (PCR) is often used for the detection, in particular the detection of a virus. As is commonly known in the art, for viruses that have an RNA genome, a further step is necessary before detection by PCR, which converts the RNA into DNA. The enzyme that catalyzes this process is reverse transcriptase (RT). The transcription of RNA into cDNA and the subsequent amplification of the genome via PCR is commonly known as RT-PCR and widely used in the art. This technique has been well-established for various human pathogenic viruses, including but not limited to Coronaviridae, such as SARS-CoV[-1] (severe acute respiratory syndrome coronavirus [1]), MERS-CoV (Middle East respiratory syndrome coronavirus) and SARS-CoV-2 (severe acute respiratory syndrome coronavirus 2).

For the detection of many pathogens, including SARS-CoV-2, the cause of the Covid-19 disease, a biological sample is obtained from the subject, e.g. by nasopharyngeal or oropharyngeal swabbing. The swab with the collected biological sample is then placed into a medium, like UTM (Universal Transport Medium; Copan) or VTM (Viral Transport Medium; CDC: https://www.cdc.gov/coronavirus/2019-ncov/downloadsViral-Transport-Medium.pdf) for transportation and sent to a laboratory for further the amplification based analysis. The nucleic acids, including the SARS-CoV-2 RNA genome if present in the collected sample, are then isolated from the collected biological sample by using a viral DNA/RNA extraction method (e.g. QlAamp Viral RNA Mini Kit; QIAGEN) followed by the detection of one or more target nucleic acids of the viral RNA genome by a specific PCR. The prior purification of the nucleic acids from the collected biological sample provides a pure eluate containing the template for the amplification reaction. Impurities and inhibitors of the amplification reaction that are contained in the biological sample as such or in the collection medium are removed in advance, thereby ensuring that the amplification reaction is not inhibited and can detect the presence or absence of the target with high sensitivity.

However, in the situation of a pandemic, such as in the current Covid-19 pandemic, where a very large number of patient samples must be analyzed in the shortest time possible this classic workflow shows significant disadvantages. The initial sample preparation comprising a nucleic acid isolation step slows down the whole diagnostic process even if the method is performed in an automated manner. This can therefore result in an undesired and inacceptable backlog in the diagnosis. Furthermore, due to the high number of samples to be processed during a pandemic, there is a risk of shortcoming of the reagents needed for the nucleic acid extraction, thus increasing the backlog even more. Therefore, there is an urgent need for reliable and sensitive methods that allow the amplification based detection of the presence or absence of a pathogen in a collected biological sample that do not require the purification of the target nucleic acids prior to performing the amplification based detection.

In view of this urgent need, several approaches were developed that aimed at circumventing the sample preparation step involving nucleic acid purification and instead aimed at detecting a pathogen such as SARS-CoV-2 directly from the transport media meaning that an aliquot of the biological sample in its transport media is directly subjected to a standard PCR reaction. This is described in a number of publications published on the pre-print servers bioRxiv (https://www.biorxiv.org/) and medRxiv (https://www.medrxiv.org/). However, not surprisingly, the components of the transport media severely inhibited the PCR resulting in a significantly increase of Ct-values and therefore inacceptable loss in sensitivity. Therefore, standard PCR compositions do not work for direct detection as it is described for example in Alcoba-Florez et al (2020) and Foomsgard ans Rosenstierne (2020).

There are also some commercial PCR Kits available which can cope with the direct application of the transport media. However, they still require time consuming manual pre-processing steps of the original sample such as heating followed by centrifugation (Takara; PrimeDirect Probe RT-qPCR Mix) or addition of pretreatment solutions followed by heating (Shimadzu; 2019 Coronavirus Detection Kit). Another restriction of these systems is that even after the pre-processing steps only a limited amount of sample can be applied which may also lead to reduced sensitivity compared to PCR detection after viral RNA purification.

Previous studies also investigated the influence of an initial heat inactivation step on the PCR sensitivity and accuracy. These studies consistently confirmed that heating of the samples prior to SARS-CoV-2 detection by PCR results in dramatically increased Ct values and thus in decreased detection rates independent of whether viral nucleic acids were extracted subsequent to the heating step or not and also independent of the assay used (E, N, ORF1 ab) (Zou et al., 2020; Alcoba-Florez et al., 2020; Fomsgaard & Rosenstierne, 2020). This loss in sensitivity is especially problematic in case the viral load is low. Furthermore, from these previous reports it follows that even though a signal can be detected and time can be saved when the nucleic acid extraction step is omitted, these direct methods are not comparable with respect to the PCR sensitivity and accuracy to the established standard methods that incorporate a viral nucleic acid extraction step which is currently the gold standard in the field of SARS-CoV-2 detection (Alcoba-Florez et al., 2020; Fomsgaard & Rosenstierne, 2020).

It is thus an object of the present invention to avoid the drawbacks of the prior art and provide improved technologies, such as methods and kits, for the amplification based detection of target nucleic acids comprised in a biological sample.

It was also an object to provide a method that enables the detection of the presence or absence of a pathogen, such as a SARS virus, using an amplification based method that avoids a prior nucleic acid purification while ensuring a good sensitivity in the amplification reaction. There is in particular a need for rapid methods that avoid time consuming pretreatment steps. It is thus a further object of the present invention to also provide a rapid, direct amplification based method that does not require purification of the pathogen nucleic acid, such as SARS-CoV-2 RNA and avoids complicated pre-processing steps. In embodiments, such method should furthermore allow the addition of as much of the crude biological sample as possible directly to the amplification reaction without compromising the detection sensitivity.

### SUMMARY OF THE INVENTION

The present invention overcomes core drawbacks of the prior art. In particular, the present invention provides methods and kits that provide a solution to the aforementioned problems and difficulties as is demonstrated by the examples and explained herein.

In particular, rapid methods and useful kits are provided that allow the direct detection of target nucleic acids, in particular target nucleic acids derived from a pathogen, from crude biological samples, including samples contained in transport medium, without the need for prior extraction of the target nucleic acid. The technology described herein is based on the pretreatment of the crude biological sample with a specifically designed extraction composition that prepares the biological sample for direct amplification without prior nucleic acid purification. As is demonstrated by the examples, using the pretreatment technology of the present invention improves the sensitivity in direct amplification protocols. The direct amplification technology disclosed herein is rapid and avoids not only a nucleic acid purification prior to amplification, it also omits time consuming or sample compromising pre-processing steps such as filtration or centrifugation. The pretreatment protocol disclosed herein furthermore allows subjecting large amount of the pretreated crude biological sample into the amplification reaction, whereby the sensitivity of the subsequent amplification may be increased. The methods according to the present invention are compatible with standard thermocycling and isothermal amplification procedures and advantageously may be performed in a single reaction vessel. Thereby, the benefits of standard amplification, such as standard reverse-transcription amplification, such as quantitative reverse-transcription PCR, are combined with the benefits of direct detection. Because of its rapidness and straightforward workflow, the methods and kits according to the present invention are particularly suitable for the processing of a large number of crude biological samples for rapid pathogen detection, as it is e.g. required during pandemic situations. The present invention thereby greatly improves the detection of pathogens in biological samples. As is shown in the present examples, the method is particularly suitable for detecting the presence or absence of RNA viruses such as SARS-CoV-2, in respiratory samples, such as swab samples. The present invention therefore makes an important contribution to the art.

According to a first aspect, a method is provided for preparing a biological sample for amplification based detection of at least one target nucleic acid comprised in the biological sample without prior target nucleic acid purification, comprising
contacting the biological sample with an extraction composition comprising
   (a) at least one surfactant,
   (b) at least one nuclease inhibitor, and
   (c) optionally at least one reducing agent, and
incubating the admixture comprising the biological sample in contact with the extraction composition to provide the prepared biological sample for amplification based detection of the target nucleic acid.

The method according to first aspect is particularly suitable preparing a crude biological sample, such as a biological medium contained in transport medium, for the direct amplification based detection of the presence of absence of a pathogen in a biological sample. As disclosed herein, the pathogen may be an RNA virus, such as in particular a coronavirus. As disclosed herein, the method is particularly suitable for preparing a biological sample for the detection of the presence or absence of SARS-CoV-2 in a biological sample, such as respiratory specimens.

According to a second aspect, a method is provided for amplification based detection of at least one target nucleic acid comprised in a biological sample without prior purification of the target nucleic acid, comprising
(A) preparing the biological sample for amplification based detection of the target nucleic acid, wherein preparing comprises
   contacting the biological sample with an extraction composition comprising
      (a) at least one surfactant,
      (b) at least one nuclease inhibitor, and
      (c) optionally at least one reducing agent, and
   incubating the admixture comprising the biological sample in contact with the extraction composition to provide the prepared biological sample;
(B) subjecting at least an aliquot or all of the prepared biological sample to an amplification reaction and amplifying the at least one target nucleic acid, optionally wherein a reverse transcription reaction is performed in order to reverse transcribe RNA to cDNA prior to amplification. As disclosed herein, step (A) is preferably performed using the method according to the first aspect described herein. Advantageously, and in contrast to many prior art methods, no nucleic acid purification is performed in advance of the amplification reaction. Instead, the crude sample that has been prepared as described herein is used in the amplification reaction.

According to a third aspect, a method is provided for detecting the presence or absence of a pathogen in a biological sample based on amplifying at least one target nucleic acid derived from the pathogen, comprising
(A) preparing the biological sample for amplification based detection of the target nucleic acid, wherein preparing comprises
   contacting the biological sample with an extraction composition comprising
      (a) at least one surfactant,
      (b) at least one nuclease inhibitor, and
      (c) optionally at least one reducing agent, and
   incubating the admixture comprising the biological sample in contact with the extraction composition to provide the prepared biological sample;
(B) subjecting at least an aliquot or all of the prepared biological sample to an amplification reaction and amplifying the at least one target nucleic acid, optionally wherein a reverse transcription reaction is performed in order to reverse transcribe RNA to cDNA prior to amplification. Again step (A) is preferably performed using the method according to the first aspect described herein. Advantageously, nucleic acid purification prior to amplification can be avoided thereby rendering the method less time and resource consuming.

The methods according to the second and third aspect are particularly suitable for detecting the presence or absence of a pathogen such as a virus in a biological sample. As disclosed herein, the pathogen may be a RNA virus, such as in particular a coronavirus. As disclosed herein, the methods are particularly suitable for detecting the presence or absence of SARS-CoV-2 in various biological samples, such as in particular respiratory specimens.

According to a fourth aspect, a kit is provided that comprises (a) the extraction composition according to the present invention. This extraction composition is disclosed in detail in conjunction with the method according to the first aspect. The kit according to the present invention is suitable for performing the method according to the first, second or third aspect. The kit may therefore also comprise instructions for performing such methods. The kit according to the fourth aspect may furthermore comprise one or more or preferably all of the following components:
(b) a DNA polymerase;
(c) a reverse transcriptase;
(d) an amplification reaction buffer comprising a Mg²⁺ source, a buffering agent and optionally further additives;
(e) a dNTP mix; and
(f) primers for amplifying the at least one target nucleic acid.

According to a fifth aspect, the present disclosure pertains to the use of a kit according to the fourth aspect in a method according to the first, second and/or third aspect.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1: Ct-thresholds for (RT-)PCR reactions with increasing amounts of Tween20. The left column (gray) indicates the results with human DNA as target, the right column (black) indicates the results with the QN IC RNA as target. The y-axis gives the number of cycles until the threshold was reached.

FIG. 2: Ct-thresholds for (RT-)PCR reactions with increasing amounts of Tween20, Tween60, and Brij58 in the reaction. Internal controls for DNA (left columns, gray) and RNA (right columns, black) were used as targets, respectively. The y-axis gives the number of cycles until the threshold was reached.

FIG. 3: Ct-thresholds for (RT-)PCR reactions without (left columns, black) and with RNase inhibitor (right columns, gray) (5000 copies of N2 target / reaction). The two negative samples were obtained from swabs of a patient with a normal throat (sample 1) and a second patient with a conspicuous throat (sample 2). The y-axis gives the number of cycles until the threshold was reached.

FIG. 4: Ct-thresholds for RT-PCR reactions without (left columns, black) or with RNase inhibitor (right columns, gray), with 5, 50, 500 copies respectively of the N2 target / reaction after incubation for 5, 10, and 30 minutes on ice. The y-axis gives the number of cycles until the threshold was reached.

FIG. 5: Ct-thresholds for RT-PCR reactions when the “extraction solution” comprising Tween20 with and without RNAse inhibitor was used in combination with heating or not for 500 and 5000 copies. The data represented on the x-axis from left to right: extraction solution (i) without heating (ice) and with RNAse inhibitor (left column in lightest gray), (ii) without heating (ice) and without RNase inhibitor (second left column), (iii) with heating at 45° C. and with RNase inhibitor (third column from left), (iv) with heating at 45° C. without RNase inhibitor (third column from right), (v) with heating at 95° C. and with RNase inhibitor (second column from right), and (vi) with heating at 95° C. and without RNase inhibitor (right last column). The y-axis gives the number of cycles until the threshold was reached.

FIG. 6: Ct-thresholds for RT-PCR reactions with different RNase inhibitors in the “extraction solution” and heating at either 45° C. or 85° C. before the RT-PCR reaction as indicated. A non-heated sample which was directly applied to the PCR without a previous incubation step was used as control (no incubation). The values 0, 500 and 5000 indicate the number of copies. Water was used as negative control. The data on the x-axis as presented from left to right: without RNase inhibitor (white) or with different RNase inhibitors (NxGen: dark grey / second left column, QIAGEN: black / third left column, Promega: light grey / right column). The y-axis gives the number of cycles until the threshold was reached. In case of the non-heated throat swab control sample with 0 copies a signal could be detected which might be explained with an unspecific amplification.

FIG. 7: Ct-thresholds of PCR (black, right column) and RT-PCR (gray, left column) amplifications in the presence of the reducing agents TCEP, N-Acetyl-L-cysteine, and DTT. The y-axis gives the number of cycles until the threshold was reached.

FIG. 8: Ct-thresholds of RT-PCR amplifications with different compositions of the “extraction solution” for 500 (gray, left column) and 5000 copies (black, right column). Numbers indicate the concentrations of the respective tested additive in the solution (see Table 2). The y-axis gives the number of cycles until threshold was reached.

FIG. 9: Ct-thresholds of RT-PCR amplifications with the three different compositions of the “extraction solution” (ES) (see Table 3). The number 10 on the x-Axis indicates that 10 µl prepared sample was used per PCR. The y-axis gives the number of cycles until the threshold was reached.

FIGS. 10: Ct-thresholds of PCR (black, left bars) and RT-PCR (gray, right bars) amplifications with and w/o heating at 95° C. and with and w/o “extraction solution” added in (a) UTM (FIG. 10a) and (b) UTM spiked with Jurkat cells (FIG. 10b). The y-axis gives the number of cycles until the threshold is reached.

FIGS. 11: Ct-differences of PCR (black, left bars) and RT-PCR (gray, right bars) amplifications between timepoint zero (0 hours) and (a) 4 hours (upper) and (b) 3 days (lower). Signals >0 are increasing Ct-values after storage indicating decreasing sample quality. The y-axis gives the number of cycle differences.

FIG. 12: Ct-thresholds for PCR (gray, left bars) and RT-PCR (black, right bars) using internal controls as targets. The volumes (in µl) indicate the amount of the respective solution (UTM, PBS) added to the PCR reaction. The y-axis gives the number of cycles until the threshold was reached.

FIGS. 13: Comparison of the PCR and RT-PCR results between the standard QN master mix (FIG. 13a) and a modified version of the QN master mix in which the alkali metal salt concentration was reduced compared to the standard QN master mix (FIG. 13b). In FIGS. 13a and 13b, Ct-thresholds for PCR (gray, left bars) and RT-PCR (black, right bars) are indicated using internal controls (DNA and RNA) as targets. The volumes (in µl) give the amount of 0.9% NaCl added to the reaction. Water was used as control. The y-axis gives the number of cycles until the threshold was reached.

FIGS. 14: Ct-thresholds for PCR (gray, left bars) and RT-PCR (black, right bars) reactions using internal controls (DNA and RNA) as targets. The volumes (in µl) indicate the amount of the respective solution. FIG. 14a shows the addition of 0.9% NaCl (compare FIG. 13b), FIG. 14b shows the addition of UTM, and FIG. 14c shows the addition of PBS to the PCR reaction. Water was used as control. The y-axis gives the number of cycles until the threshold was reached.

FIG. 15: Ct-thresholds for RT-PCR reactions targeting the SARS-CoV-2 genes N1, N2, E, RdRP, and Orf1b. The assays used were according to the sequences published from the (US) CDC, Charite and used in the QIAstat-Dx instrument (QIAGEN). The values 0, 3, 6, 12 indicate the amount of transport medium UTM (in µl) added to the PCR reaction (0 µl: black, left bar; 3 µl: light gray, second left bar; 6 µl: middle gray, second right bar; 12 µl: dark gray, right bar). Below each set of columns the primer annealing temperature for the respective signals is presented. The y-axis gives the number of cycles until the threshold was reached.

FIG. 16: Ct-thresholds for RT-PCR reactions targeting the SARS-CoV-2 E-gene (Charite assay). The values 0, 3, 6, 12 indicate the amount of the two different transport media UTM (in µl) added to the PCR reaction (UTM lot 1: dark gray, left bar; UTM lot 2: middle gray, right bar). The y-axis gives the number of cycles until the threshold was reached.

FIG. 17: Ct-thresholds for RT-PCR reactions targeting the SARS-CoV-2 N2-gene (CDC assay). The values 0, 3, 6, 12 indicate the amount of solution added to the PCR reaction. The order of the bars from left to right are in accordance with the legend top down wherein the following PCR reaction buffers were tested: (1) PCR reaction buffer with KAc/NaAc acetic acid; (2) PCR reaction buffer with KAc/NaAc HCl; (3) PCR reaction buffer with KCI/NaCI HCl; (4) PCR reaction buffer without KCI/NaCI acetic acid; (5) PCR reaction buffer without KCI/NaCI HCI. The y-axis gives the number of cycles.

FIG. 18: Advantageous workflow of the method according to the present invention including the use of an “extraction solution” to prepare the biological sample for direct amplification without prior nucleic acid isolation.

FIG. 19: Overview of workflows according to the invention particularly suitable for swab samples in transport media.

FIG. 20: Overview of workflows according to the invention particularly suitable for saliva and gargle samples. (A) Workflow without the use of an additional digestion solution and (B) workflow with the use of an additional digestion solution comprising a proteolytic enzyme and a reducing agent for greater sensitivity with these sample types.

FIG. 21: Individual Ct-values of RT-PCR reactions for simultaneous detection of different respiratory viruses in one multiplex reaction. E.g. the FluA columns represent the signals for FluA in different transport media which were detected in parallel to the signals of the other respiratory viruses in the same amplification reaction. HSC: Human Sampling Control.

FIG. 22: Amplification plots of dilution series (10^1, 10^2, 10^3, 10^4, and 10^5 copies/reaction) of SARS-CoV-2 variants T478K (A) and E484K (B), respectively, in comparison to WT and NTC (baseline indicated by arrow). The upper horizontal line indicates the threshold.

FIG. 23: Ct-values of RT-PCR amplification reactions for SARS-CoV-2 targets (grey), the internal (white) and loading control (black). The black horizontal line gives the baseline value without a reducing agent (TCEP).

FIG. 24: Ct-values of the internal control RNA to detect for inhibition when different amounts of proteases (QIAGEN protease or proteinase K) are added.

FIG. 25: Ct-values when comparing different protocol variations for the detection of SARS-CoV-2 in saliva samples (square: with digestion solution, 5 min, 95° C.; circle: 15 min, 95° C.; triangle: 30 min, 95° C.).

FIG. 26: Hit rate represents positive detected samples at different dilution points for saliva samples spiked with Zeptometrix Natrol standard following protocol (A) (heating) or (B) (with digestion solution) presented in FIG. 20.

FIG. 27: Hit rate (HR) represents positive detected samples at different dilution points for saliva and gargle samples. (A) Protocol with initial heating according to FIGS. 20(A). (B) Protocol with digestion solution according to FIG. 20(B).

FIG. 28: Ct-values of lollipop swab pools containing 10 and 20 donors, respectively, with one known positive donor. The positive donor was measured for comparison reasons (first column each).

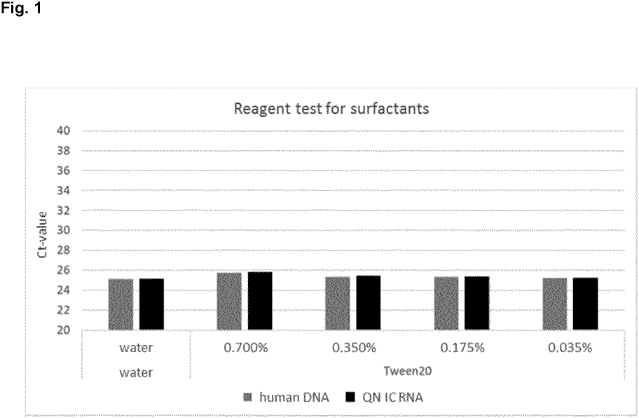

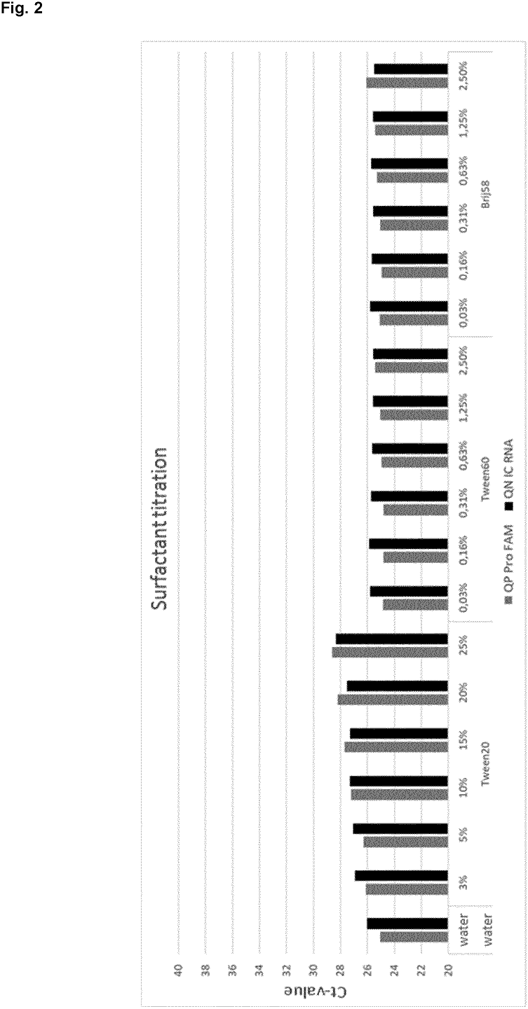

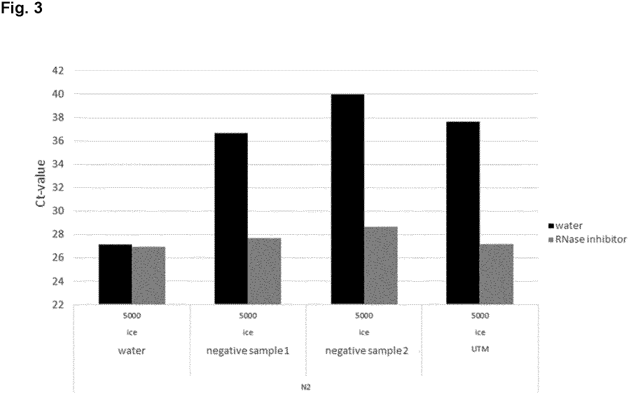

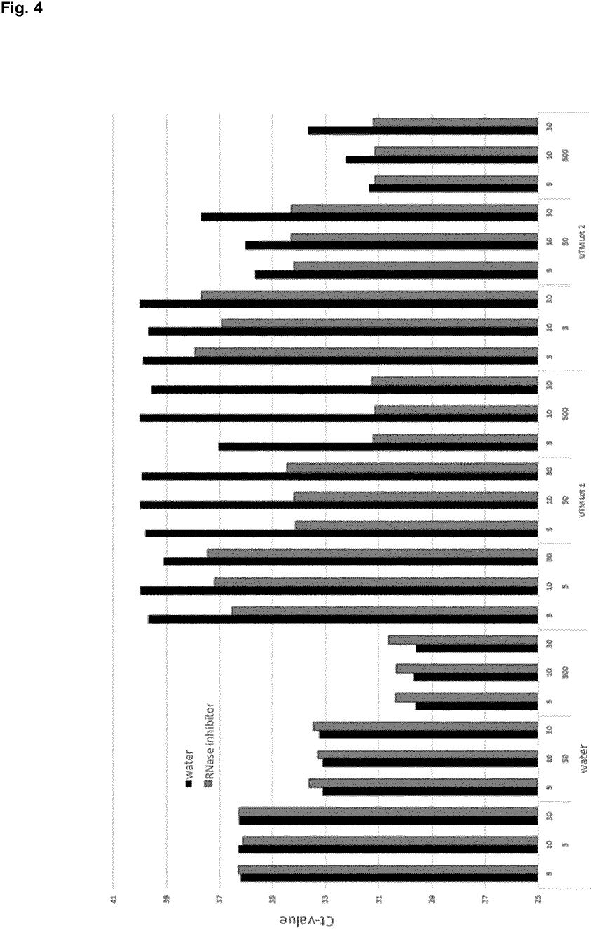

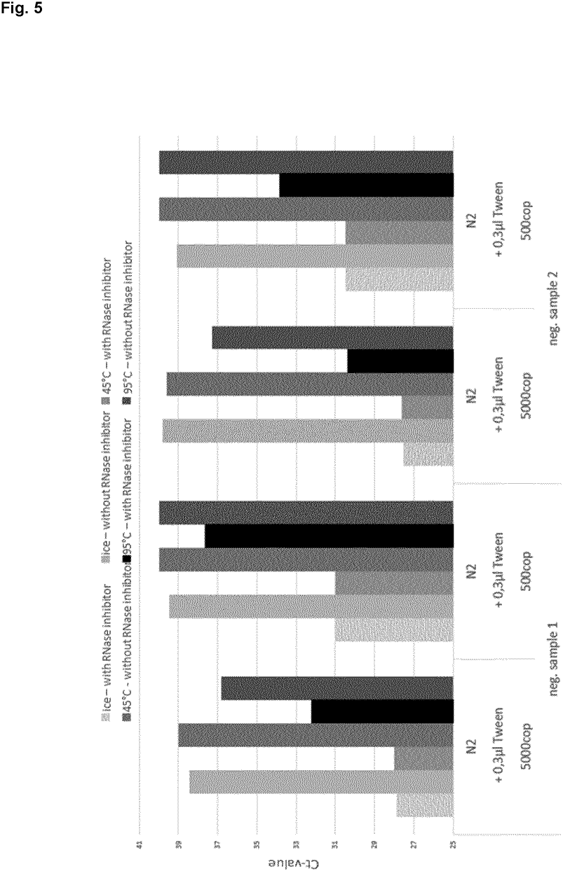

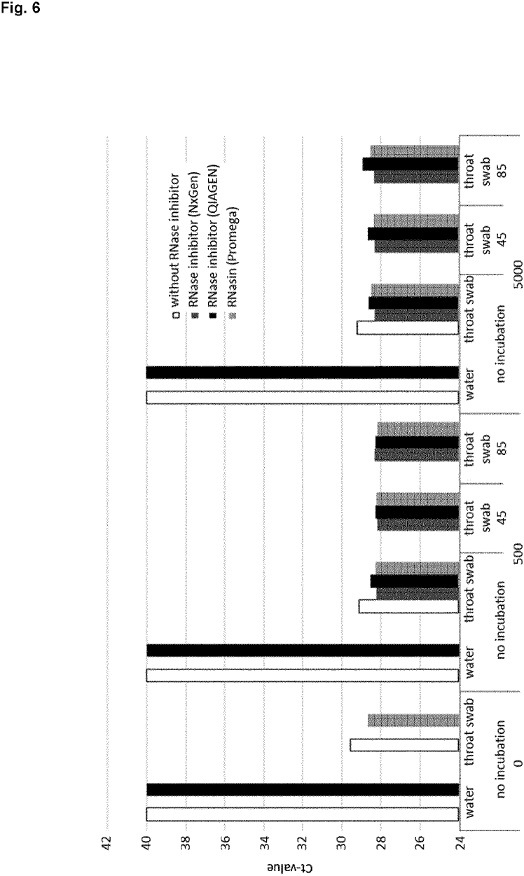

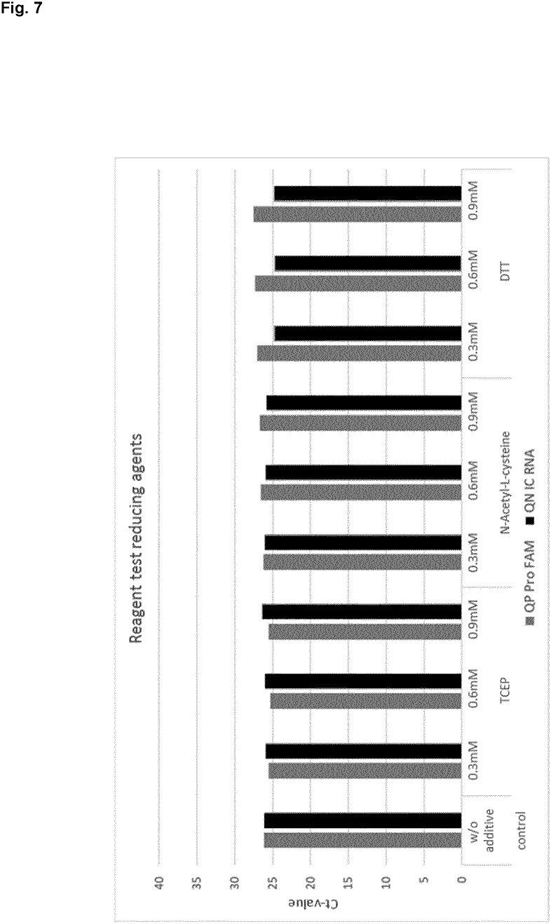

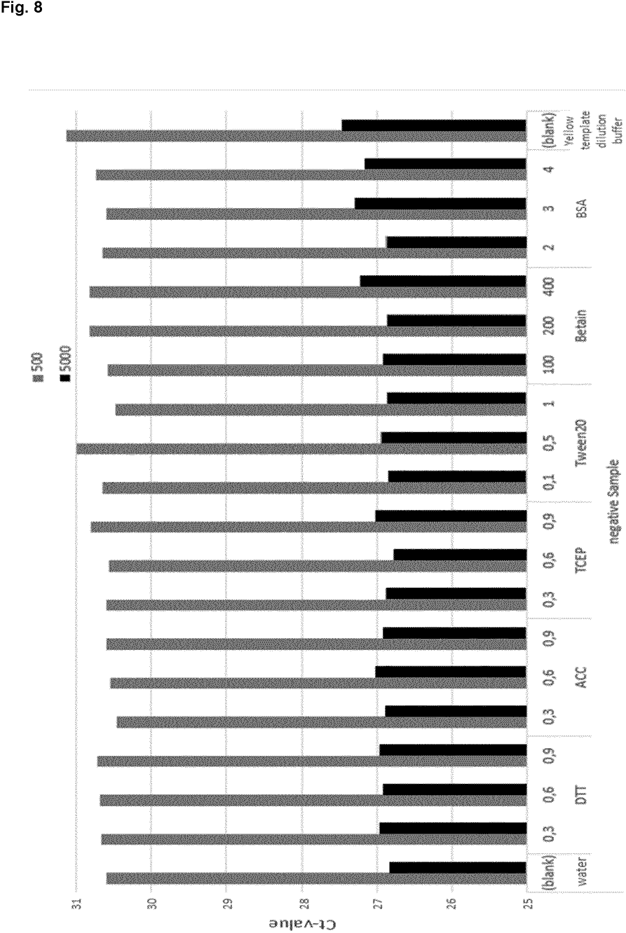

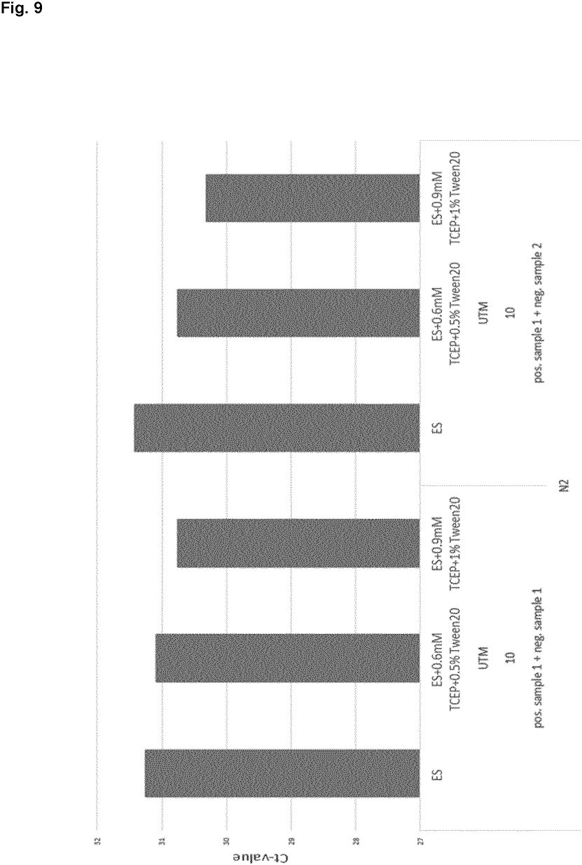

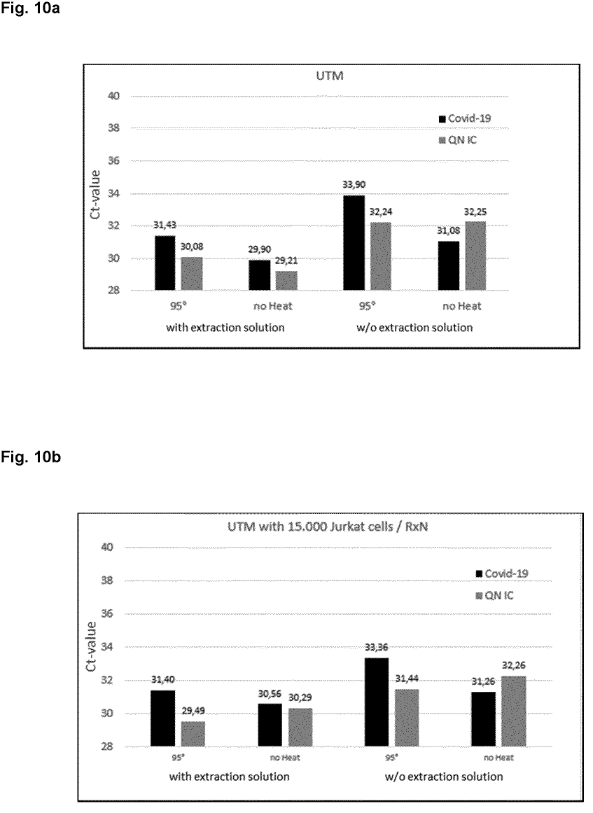

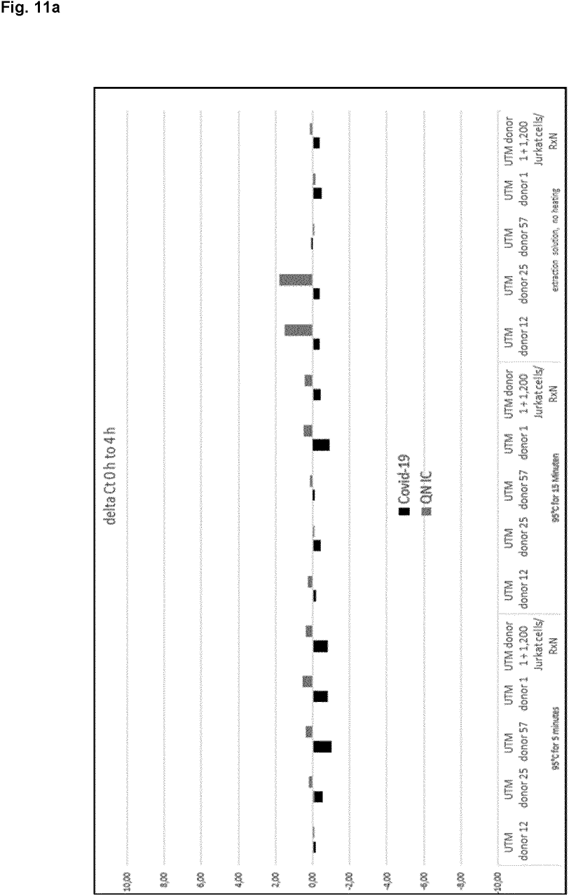

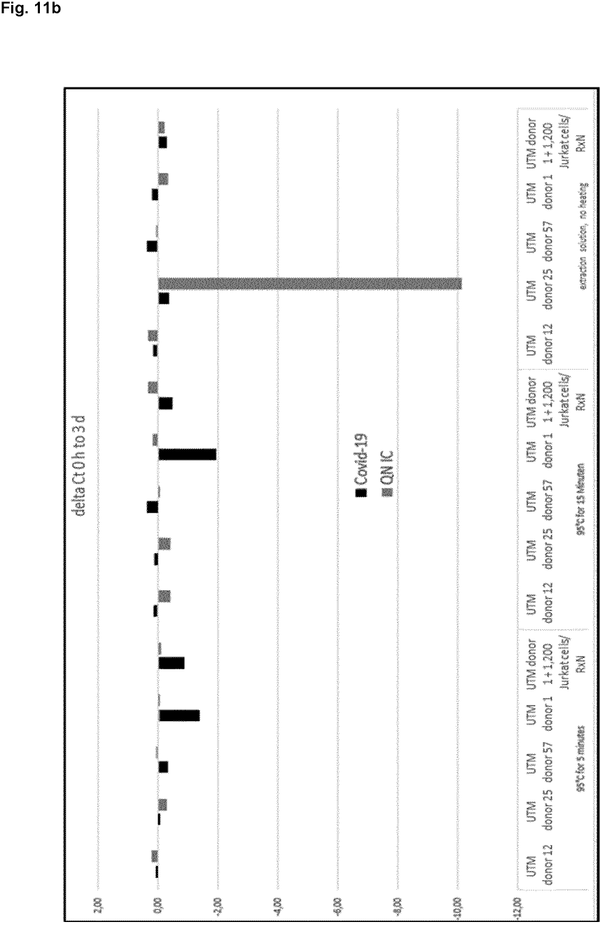

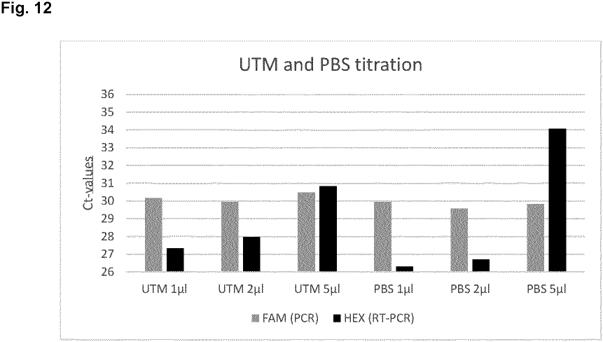

### DETAILED DESCRIPTION OF THE INVENTION

The different aspects and embodiments of the invention disclosed herein make important contributions to the art as is also explained in the following.

### THE METHOD ACCORDING TO THE FIRST ASPECT

According to a first aspect, a method is provided for preparing a biological sample for amplification based detection of at least one target nucleic acid comprised in the biological sample without prior target nucleic acid purification, comprising
contacting the biological sample with an extraction composition comprising
   (a) at least one surfactant,
   (b) at least one nuclease inhibitor, and
   (c) optionally at least one reducing agent, and
incubating the admixture comprising the biological sample in contact with the extraction composition to provide the prepared biological sample for amplification based detection of the target nucleic acid.

The individual steps and preferred embodiments will now be described in detail.

### The Extraction Composition of the Present Invention

The method according to the first aspect comprises contacting the biological sample with an extraction composition which comprises
(a) at least one surfactant,
(b) at least one nuclease inhibitor, and
(c) optionally at least one reducing agent.

The extraction composition that is contacted with the crude biological sample in order to prepare the biological sample for amplification based detection of the one or more target nucleic acids is a core aspect of the present invention. It is therefore also disclosed in isolation and may, e.g. be incorporated into the kit according to the fourth aspect of the invention. The use of such extraction composition greatly improves the results of the subsequently performed amplification reaction. As is demonstrated in the examples, the extraction composition advantageously prepares the crude biological sample for direct amplification without prior nucleic acid purification. It’s use can significantly improve the sensitivity of the subsequent amplification reaction, such as a reverse-transcription amplification reaction, as is shown by the examples. In particular, the extraction composition that is used according to the teachings of the present invention renders the target nucleic acids well accessible for the subsequent direct amplification reaction. This by supporting the lysis of the biological sample, including e.g. contained cells and/or virus particles containing the target nucleic acids, whereby the target nucleic acids (if present in the biological sample) are rendered accessible e.g. for the primers and enzymes that are used in the subsequent amplification reaction, which in a preferred embodiment is a reverse transcription amplification reaction. At the same time the extraction composition effectively inhibits the undesired degradation of the target nucleic acids by nucleases in the so prepared biological sample. This is particularly advantageous in case of RNA target nucleic acids, because RNA, including viral RNA, is particularly prone to degradation by RNases that are e.g. released from the eukaryotic cells additionally contained in the biological sample or the medium that contains the actual biological sample (the biological sample that is contacted with the extraction composition is in a core embodiment provided by a biological sample comprised in a collection/transport medium). It is therefore particularly important to protect the target RNA from degradation. This particularly if aiming at detecting a pathogenic RNA target nucleic acid (e.g. derived from a RNA virus such as a coronavirus) as there is otherwise a risk of false negative results.

The components comprised in the extraction composition achieve in combination the above mentioned beneficial effects in preparing the crude biological sample for direct amplification and advantageously do not interfere with each other or the subsequent direct amplification reaction (which in a preferred embodiment is a reverse transcription amplification reaction). The extraction composition of the present invention that is used for preparing the crude biological sample for direct amplification without prior nucleic acid purification is thus compatible with standard amplification and reverse transcription amplification methods.

The individual components of the extraction composition and preferred embodiments thereof are described in the following. As disclosed herein, the extraction composition is preferably an extraction solution. All disclosures and embodiments described in this application for the extraction composition in general, specifically apply and particularly refer to the preferred embodiment of using an extraction solution even if not explicitly stated.

### The Surfactant Comprised in the Extraction Composition

The extraction composition comprises at least one surfactant. The comprised surfactant supports the lysis of the crude biological sample, including contained cells and virus particles. The surfactant-induced lysis thereby assists in releasing the target nucleic acids, such as e.g. viral nucleic acids, and thereby renders them accessible for amplification/reverse transcription. The surfactant that is comprised in the extraction composition does not interfere with the subsequent enzymatic reaction (such as the amplification and/or reverse amplification). This at least in the concentration in which it is included into the enzymatic reaction via the prepared biological sample that comprises the biological sample and components of the extraction composition.

The surfactant may be selected from non-ionic and amphoteric surfactants. According to an advantageous embodiment, the surfactant is a non-ionic surfactant. As is demonstrated by the examples, different non-ionic surfactants may be used in conjunction with the present invention. According to one embodiment, the non-ionic surfactant is a polyoxyethylene-based non-ionic surfactant. It may be selected from the group consisting of polyoxyethylene fatty acid esters, in particular polyoxyethylene sorbitan fatty acid esters; polyoxyethylene fatty alcohol ether; polyoxyethylene alkylphenyl ethers; and polyoxyethylene-polyoxypropylene block copolymers. In advantageous embodiments, the non-ionic surfactant comprised in the extraction composition is selected from polyoxyethylene fatty acid esters, in particular polyoxyethylene sorbitan fatty acid esters, and polyoxyethylene fatty alcohol ethers.

According to one embodiment, the extraction composition comprises a polyoxyethylene fatty acid ester, comprising
a fatty acid derived from laureate, palmitate, stearate and oleate,
a polyoxyethylene component containing from 2 to 150, 4 to 100, 6 to 50 or 6 to 30 (CH₂CH₂O) units.

Polyoxyethylene sorbitan fatty acid esters, also referred to as polysorbates, are particularly preferred as non-ionic surfactant for the extraction composition. The non-ionic surfactant may be selected from polyoxyethylene (20) sorbitan monolaurate (e.g. Tween20), polyoxyethylene (4) sorbitan monolaurate (e.g. Tween21), polyoxyethylene (40) sorbitan monopalmitate (e.g. Tween40), polyoxyethylene (60) sorbitan monostearate (e.g. Tween60), polyoxyethylene (4) sorbitan monostearate (e.g. Tween61), polyoxyethylene (20) sorbitan tristearate (e.g. Tween65), polyoxyethylene (40) sorbitan monooleate (e.g. Tween80), polyoxyethylene (5) sorbitan monooleate (e.g. Tween81), polyoxyethylene sorbitan trioleate (e.g. Tween85), and polyoxyethylen (20) sorbitan monoisostearate. According to a preferred embodiment, the polyoxyethylene fatty acid ester is selected from polysorbate 20, polysorbate 40, polysorbate 60 and polysorbate 80. As is demonstrated in the examples, such non-ionic surfactants are advantageous because they assist the lysis and thus preparation of the crude biological sample and do not inhibit the subsequent amplification reaction (PCR and RT-PCR) even when used in higher concentrations. The use of polysorbate 20 is particularly preferred.

According to one embodiment the extraction composition comprises as non-ionic surfactant a polyoxyethylene fatty alcohol ether, comprising
a fatty alcohol component having from 6 to 22 carbon atoms, and
a polyoxyethylene component containing from 2 to 150, 4 to 100, 6 to 50 or 6 to 30 (CH₂CH₂O) units.

The polyoxyethylene fatty alcohol ether may be selected from the group consisting of polyoxyethylene lauryl ether, polyoxyethylene cetyl ether, polyoxyethylene stearyl ether and polyoxyethylene oleyl ether. In a preferred embodiment, the polyoxyethylene fatty alcohol ether is selected from the group comprising polyoxyethylene cetyl ether, polyoxyethylene stearyl ether and/or polyoxyethylene oleyl ether. Suitable examples include but are not limited to polyoxyethylene cetyl or polyoxyethylene oleyl alcohol ethers, such as polyoxyethylene(10) cetyl ether (Brij® 56), polyoxyethylene(20) cetyl ether (Brij® 58) and polyoxyethylene(20) oleyl ether (Brij® 98).

In one embodiment the extraction composition comprises as non-ionic surfactant a polyoxyethylene alkyl phenyl ether. The polyoxyethylene alkylphenyl ether may have an alkyl group containing from five to 15 carbon atoms, such as 6 to 10 carbon atoms. Also encompassed are branched or unbranched C₇- to C₁₀-alkyl groups, such as branched or unbranched C₈- and C₉-alkyl groups, e.g. isooctyl groups and nonyl groups. The polyoxyethylene alkylphenyl ether may be selected from the group consisting of polyoxyethylene nonylphenyl ether and polyoxyethylene isooctylphenyl ether. It may be Triton X 100.

Polyoxyethylene-polyoxypropylene block copolymers may also be included as non-ionic surfactant in the extraction composition. Polyoxyethylene-polyoxypropylene block copolymers are also referred to as “poloxamers”. Polyoxyethylene-polyoxypropylene block copolymers may be of the empirical formula HO(C₂H₄O)ₐ(C₃H₆O)_{b}(C₂H₄O)ₐH, where “a” refers to the number of polyoxyethylene units and “b” refers to the number of polyoxypropylene units, with the a/b weight ratio optionally being in the range from 0.1 to 3. Such polyoxyethylene-polyoxypropylene block copolymers can be obtained, for example, under the trade name Pluronic® or Synperonic®.

According to one embodiment, the crude biological sample is contacted with an extraction solution that comprises the surfactant, preferably a non-ionic surfactant as described above, in a concentration that lies in a range of 0.1% to 30% (w/v). Suitable ranges include but are not limited to 0.5% to 25% (w/v), 0.7% to 20% (w/v) and 1% to 15% (w/v). In further embodiments the surfactant concentration in the extraction solution is 1.2% to 10% (w/v), 1.5% to 8% (w/v) or 2% to 5% (w/v). Following the teachings of the present application, the skilled person can chose suitable surfactant concentrations for the extraction solution also depending on the amount of crude biological sample to be contacted with the extraction solution. To ensure that the prepared biological sample comprises a high amount of the original biological sample (which is optionally contained in medium) it is advantageous to use a concentrated extraction solution. This allows using a small amount of extraction solution in order to prepare a larger amount of crude biological sample. E.g. the extraction solution may be concentrated 3x or 5x. In other embodiments, the extraction solution is concentrated 10x, 15x or 20x. Hence, the concentration factor of the extraction solution may be in the range of 3x to 20x.

In advantageous embodiments, the resulting admixture that is prepared by contacting the biological sample (optionally contained in medium) with the extraction composition comprises the surfactant, preferably a non-ionic surfactant as described above, in a concentration that lies in a range of 0.075% to 20% (w/v). Suitable final concentration ranges for the surfactant, preferably a non-ionic surfactant, in the prepared admixture include but are not limited to 0.1% to 15% (w/v), 0.15% to 15% (w/v), 0.2% to 10% (w/v) and 0.25% to 8% (w/v). In further embodiments, the final surfactant concentration in the prepared admixture is 0.2% to 5% (w/v), 0.25% to 3% (w/v) or 0.3% to 2% (w/v).

According to a further embodiment, the surfactant is an amphoteric surfactant. The amphoteric surfactant may be a betaine such as N,N,N trimethylglycine. As is demonstrated by the examples, betaine does not interfere with the subsequent amplification reaction and is also compatible with the further components of the extraction composition, such as the proteinaceous RNase inhibitor that is preferably included in case of RNA target nucleic acids such as viral RNA target nucleic acids. In one embodiment the extraction solution comprises the amphoteric surfactant such as a betaine in a concentration lies in the range of 50 mM to 1 M, such as 100 mM — 500 mM.

The extraction solution may also comprise two or more surfactants, preferably selected from non-ionic surfactants and amphoteric surfactants.

### The Nuclease Inhibitor Comprised in the Extraction Composition

The extraction composition lyses the biological sample, including contained pathogens of interest such as viral particles, and at the same time inhibits nucleases that could degrade the target nucleic acids, such as target RNA or target DNA. It was found that biological samples as described herein, such as swab samples comprised in transport media, often contain high amounts of nucleases. The nucleases may origin from the comprised eukaryotic cells but also from undefined media components, such as fetal calf serum that may be comprised in standard swab transport medium.

To inhibit the degradation of target nucleic acids by nucleases in the prepared biological sample, the extraction composition comprises at least one nuclease inhibitor. It is preferred that the nuclease inhibitor achieves strong nuclease inhibition, in order to effectively protect the target nucleic acids that are released by the surfactant containing extraction composition from nuclease degradation. As disclosed herein, nucleases may also be released from the cells that are lysed by the extraction composition.

The nuclease inhibitor may be an RNase inhibitor or a DNase inhibitor. The extraction solution comprises a nuclease inhibitor that is capable of protecting the target nucleic acid of interest. The extraction composition may also comprise two or more nuclease inhibitors, such as (i) two or more RNase inhibitors, (ii) two or more DNase inhibitors or (iii) one or more RNase inhibitors and one or more DNase inhibitors. Using an extraction composition comprising a RNase inhibitor as well as a DNase inhibitor can be advantageous in order to provide a universal extraction composition and thus universal preparation method that is compatible with RNA and DNA target nucleic acids.

The nuclease inhibitor that is comprised in the extraction composition does not interfere with the subsequent enzymatic reaction (such as the amplification and/or reverse amplification) at least in the concentration in which it is included into the enzymatic reaction via the prepared biological sample that comprises the biological sample and components of the extraction composition. Hence, a reverse transcription reaction and/or an amplification reaction can be performed in the presence of the comprised nuclease inhibitor.

According to one embodiment, the nuclease inhibitor is a RNAase inhibitor. As is demonstrated by the examples, incorporating a RNase inhibitor into the extraction composition that is used for preparing the biological sample greatly improves the results of a subsequently performed direct reverse transcription amplification to which the prepared biological sample is subjected without prior nucleic acid purification. As disclosed herein, the target nucleic acid is in preferred embodiments a RNA, such as a viral RNA. Therefore, preventing degradation of the viral RNA is particularly advantageous to increase the sensitivity of the virus detection. The RNase inhibitor may have broad-spectrum RNase inhibitory properties and may inhibit RNase A, B and C as well as human placental RNase. It does not inhibit the reverse transcriptase or the DNA polymerase used, such as Taq polymerase.

The use of a strong RNase inhibitor is preferred in order to maximize the protection of the target RNA from degradation. Strong RNase inhibitors are well-known and are often provided by proteins, in particularly recombinantly produced proteinaceous RNase inhibitors. In an advantageous embodiment, the RNase inhibitor is thus a proteinaceous RNase inhibitor. Numerous proteinaceous RNase inhibitors are commercially available and can thus be used in conjunction with the present invention as is also demonstrated in the examples. Examples of proteinaceous RNase inhibitors include, but are not limited to, QIAGEN RNase Inhibitor, RNasin® Ribonuclease Inhibitor, NxGen RNase inhibitor and others.

The amount/concentration of the RNase inhibitor in the extraction composition of the present invention can be experimentally determined by the skilled person following the guidance provided in the application and e.g. the manufacturer instructions for the chosen RNase inhibitor. Incorporating more of the RNase inhibitor will usually achieve a stronger RNase inhibitory effect.

In one embodiment, where RNA target nucleic acids are of interest, the extraction composition comprises a RNase inhibitor, preferably a proteinaceous RNase inhibitor, but does not comprise a separate DNase inhibitor. In this embodiment, the RNA target nucleic acids are protected from degradation by the RNase inhibitor, while any degradation of non-target DNA would reduce the non-target nucleic acid background. Corresponding considerations apply where DNA target nucleic acids are of interest, and wherein the extraction composition comprises a DNase inhibitor but does not comprise a separate RNase inhibitor.

### The Reducing Agent Comprised in the Extraction Composition

In a preferred embodiment, the extraction composition comprises a reducing agent. Incorporating a reducing agent is advantageous as it assists the preparation of the biological sample for direct amplification.

The reducing agent preferably supports the destruction of disulfide bonds and denaturation of proteins comprised in the biological sample. The reducing agent can thus assist in the inhibition of the nucleases. It can furthermore support the release of the target nucleic acids. Furthermore, incorporating a reducing agent into the extraction composition is advantageous because it can assist to liquefy the biological sample. This can simplify the processing of viscous biological samples, such as respiratory samples. Liquefying a viscous biological sample is advantageous because the target nucleic acids are better accessible in a liquefied biological sample and the prepared biological sample is more homogeneous. Reducing agents are known in the art. The reducing agent that is comprised in the extraction composition does not interfere with the subsequent enzymatic reaction (such as the amplification and/or reverse amplification) at least in the concentration in which it is included into the enzymatic reaction via the prepared biological sample that comprises the biological sample and components of the extraction composition. Hence, a reverse transcription reaction and/or an amplification reaction can be performed in the presence of the comprised reducing agent.

In one embodiment, the reducing agent is selected from Tris(carboxyethyl)phosphine (TCEP), Dithiothreitol (DTT), N-acetyl cysteine, THPP (Tris(hydroxypropyl)phosphine), 1-thioglycerol and beta-mercaptoethanol. In one embodiment, the comprised reducing agent is selected from Tris(carboxyethyl)phosphine (TCEP), Dithiothreitol (DTT), N-acetyl cysteine, THPP (Tris(hydroxypropyl)phosphine) and 1-thioglycerol. In one embodiment, the comprised reducing agent is selected from Tris(carboxyethyl)phosphine (TCEP), Dithiothreitol (DTT) and N-acetyl cysteine. As is demonstrated in the examples, these reducing agents do not interfere with the subsequent reverse transcription reaction and/or an amplification reaction. In a particular embodiment, the extraction composition comprises Tris(carboxyethyl)phosphine (TCEP) as reducing agent. TCEP is storage stable and therefore, is advantageous for ready-to-use kit formats.

As is demonstrated in the examples, an extraction composition comprising in addition to the RNase inhibitor and surfactant a reducing agent such as TCEP further improves the subsequently performed direct amplification reaction to which the prepared biological sample is subjected.

In one embodiment, the extraction composition comprises the reducing agent in a concentration that lies in a range of 0.3 mM to 50 mM. Suitable concentration ranges for the reducing agent include but are not limited to 0.5 mM to 25 mM, 1 mM to 20 mM and 1.5 mM to 15 mM. In embodiments, the extraction composition comprises the reducing agent in a concentration in a range of 2 mM to 10 mM or 2 mM to 5 mM. The extraction composition comprises in one embodiment a reducing agent that is selected from Tris(carboxyethyl)phosphine (TCEP), Dithiothreitol (DTT), N-acetyl cysteine, THPP (Tris(hydroxypropyl)phosphine) and 1-thioglycerol in a concentration that lies in the range of 1 mM to 10 mM or 2 mM to 5 mM, wherein in a preferred embodiment the reducing agent is TCEP. Following the teachings of the present application, the skilled person can chose a suitable reducing agent concentration for the extraction composition. E.g. in case of more viscous biological samples, the concentration may be increased to further support the rapid liquefaction of the biological sample, also when the biological sample is contained in medium. As noted above, to ensure that the prepared biological sample comprises a high amount of the original biological sample (which is optionally contained in medium) it is advantageous to use a concentrated extraction solution. This allows using a small amount of extraction solution in order to prepare a larger amount of crude biological sample. E.g. the extraction solution may be concentrated 3x or 5x. In other embodiments, the extraction solution is concentrated 10x, 15x or 20x. Hence, the concentration factor of the extraction solution may be in the range of 3x to 20x.

In advantageous embodiments, the resulting admixture that is prepared by contacting the biological sample (optionally contained in medium) with the extraction composition comprises the reducing agent in a concentration that lies in a range of 0.1 mM to 15 mM. Suitable concentration ranges for a reducing agent such as TCEP in the prepared admixture include but are not limited to 0.2 mM to 10 mM, 0.25 mM to 8 mM and 0.3 mM to 5 mM. In further embodiments, the final reducing agent concentration in the prepared admixture is 0.35 mM to 2 mM or 0.4 mM to 1.5 mM.

### Embodiments of the Extraction Composition

As noted above, in preferred embodiments the extraction composition is provided as liquid composition. The use of an extraction solution is advantageous because such solution can be easily mixed with the crude biological sample, which in preferred embodiments is a biological sample comprised in medium. The active components of the extraction solution, i.e. the nuclease inhibitor (preferably a proteinaceous RNase inhibitor), the surfactant (preferably a non-ionic surfactant) and the preferably comprised reducing agent, can be quickly dispersed in the biological sample and can thereby ensure the efficient lysis and preparation of the biological sample and protection of the target nucleic acids. This process can be assisted by agitation, such as vortexing, to ensure that the extraction solution and the biological sample are mixed well.

Particularly advantageous extraction solutions suitable to prepare a crude biological sample such as a biological sample contained in medium for direct reverse transcription and amplification of the target nucleic acids without prior purification of the contained nucleic acids are described in the following. As is demonstrated by the examples, accordingly designed extraction solutions achieve particularly favorable results. In embodiments, the subsequently described extraction solutions consist essentially of or consist of the carrier liquid (which may comprise a buffering agent or can be unbuffered) of the extraction solution and the identified active ingredients.

According to one embodiment, the extraction solution comprises
(a) at least one non-ionic surfactant,
(b) at least one proteinaceous RNase inhibitor, and
(c) at least one reducing agent.

Suitable and preferred embodiments of the non-ionic surfactant and the reducing agent were described above and it is referred to the respective disclosure.

According to one embodiment, the extraction solution comprises
(a) at least one polyoxyethylene-based non-ionic surfactant,
(b) at least one proteinaceous RNase inhibitor, and
(c) at least one reducing agent selected from Tris(carboxyethyl)phosphine (TCEP), Dithiothreitol (DTT), N-acetyl cysteine, THPP (Tris(hydroxypropyl)phosphine) and 1-thioglycerol.

As disclosed herein, the active ingredients of the extraction solution may consist essentially of or may consist of
(a) a non-ionic surfactant, preferably a polyoxyethylene-based non-ionic surfactant,
(b) a proteinaceous RNase inhibitor, and
(c) a reducing agent, preferably selected from Tris(carboxyethyl)phosphine (TCEP), Dithiothreitol (DTT), N-acetyl cysteine, THPP (Tris(hydroxypropyl)phosphine) and 1-thioglycerol.

Suitable and preferred embodiments of the one polyoxyethylene-based non-ionic surfactant were described above and it is referred to the respective disclosure. In advantageous embodiments, the non-ionic surfactant comprised in the extraction solution is selected from polyoxyethylene fatty acid esters, in particular polyoxyethylene sorbitan fatty acid esters, and polyoxyethylene fatty alcohol ethers.

According to a preferred embodiment, the extraction solution comprises
(a) at least one polysorbate,
(b) at least one proteinaceous RNase inhibitor, and
(c) Tris(carboxyethyl)phosphine (TCEP).

As is demonstrated by the examples, such extraction solution is very advantageous and allows to prepare even difficult biological samples, including respiratory specimens comprised in medium, for direct reverse transcription and amplification of comprised RNA target nucleic acids (such as viral RNA targets) with favorable sensitivity. Suitable polysorbates that can be included as non-ionic surfactant are disclosed above and it is referred to the respective disclosure. As described, the polysorbate may be selected from polysorbate 20, polysorbate 40, polysorbate 60 and polysorbate 80. Polysorbate 20 is a particularly preferred polysorbate that can be included in the extraction solution as non-ionic surfactant. In one embodiment, the active ingredients of the extraction solution may consist essentially of or may consist of
(a) the polysorbate (e.g. polysorbate 20),
(b) the proteinaceous RNase inhibitor, and
(c) Tris(carboxyethyl)phosphine (TCEP).

The extraction solution may have a pH in the range of 6.0 to 9.0, such as 6.0 to 8.5 or 6.3 to 8.0. The pH may be in the range of 6.5 to 7.5, such as about 7.0.

The extraction solution may in embodiments comprise a buffering agent. If a buffering agent is incorporated, it preferably does not comprise any ions that could have a negative effect on the subsequent amplification.

In an advantageous embodiment, the extraction solution is unbuffered.

The extraction composition should not comprise ingredients in a concentration that could inhibit the subsequently performed amplification/reverse transcription amplification of the one or more target nucleic acids when the prepared biological sample, that comprises the components of the extraction composition, is subjected to the amplification reaction/reverse transcription amplification reaction. Furthermore, the extraction composition should not comprise ingredients that counteract or damage the comprised core ingredients i.e. the surfactant, the nuclease inhibitor and, if comprised, the reducing agent. It is thus advantageous if the extraction composition/extraction solution does not comprise one or more, two or more, three or more or all of the following components:
an ionic surfactant;
a chaotropic salt;
chloride ions in a concentration exceeding 20 mM or exceeding 10 mM, wherein preferably the extraction solution does not comprise chloride ions (this embodiment is particularly advantageous if the target nucleic acid is RNA);
an aliphatic C1-C5 alcohol; and/or
a proteinase enzyme.

The components of the extraction composition/extraction solution are comprised in the prepared biological sample and are thus transferred to the subsequent amplification reaction, which preferably is a reverse transcription amplification reaction. It is therefore advantageous to design the extraction solution as simple as possible. In preferred embodiments, the active ingredients of the extraction composition, respectively the extraction solution, therefore consist essentially of or consist of (a) a surfactant, preferably a non-ionic surfactant, (b) the nuclease inhibitor and (c) the reducing agent, if comprised. For preparing biological samples for a subsequently performed direct reverse transcription amplification reaction (without prior nucleic acid purification) the nuclease inhibitor is as described herein a RNase inhibitor, preferably a proteinaceous RNase inhibitor.

### Further Features and Embodiments of the Method According to the First Aspect

Preparation of the admixture may comprise agitating the biological sample in contact with the extraction composition to ensure a thorough admixture of the crude biological sample and the extraction composition. For agitation, the admixture may e.g. be aspirated and dispensed and/or vortexted.

The admixture that is prepared by contacting the crude biological sample with the extraction composition according to the present invention is preferably incubated so that the ingredients comprised in the extraction composition can digest the biological sample, while protecting the target nucleic acids. In embodiments, incubation occurs for 1 to 60 min, 1 to 30 min or 1 to 20 min. In further embodiments, incubation occurs for 1 to 15 min or 1 to 10 min. As is demonstrated in the examples, the extraction composition according to the present invention is highly effective, so that very short incubation times of 1.5 to 5 min or 1.5 to 3 min, such as 2 min, are sufficient in order to prepare a crude biological sample for direct amplification. This is highly advantageous because it significantly shortens the processing time compared to workflows that require nucleic acid purification prior to amplification or incorporate other more time consuming preparation steps. However, also longer incubation times are feasible without compromising the quality of the target nucleic acids because target degradation is effectively reduced with the extraction composition of the present invention. This is highly advantageous where a high number of biological samples are processed in parallel. The biological samples first contacted with the extraction composition may simply be incubated for a longer time without compromising the quality of the prepared biological sample until the last biological samples were also contacted with the extraction composition and incubated for an appropriate time. The preparation protocol of the present invention is therefore very robust and ensures uniform results even if the incubation time varies between prepared biological samples.

Advantageously, the steps of contacting the biological sample with the extraction composition and incubating the admixture may be carried out at ambient temperature (e.g. room temperature) as is demonstrated in the examples. In embodiments, all preparation steps apart from the enzymatic reaction (amplification or reverse-transcription amplification) are carried out at ambient temperature. This simplifies the performance of the method according to the present invention. If desired, these steps may also be carried out on ice as is also shown in the examples.

In one embodiment, the method for preparing the biological sample for amplification based detection of the target nucleic acid does not involve heating the biological sample in contact with the extraction composition to a temperature ≥ 75° C., such as ≥ 70° C., ≥ 65° C., ≥ 60° C., ≥ 55° C., ≥ 50° C., ≥ 45° C. or ≥ 40° C. for at least 2 min prior to subjecting at least an aliquot or all of the prepared biological sample to the subsequent enzymatic reaction selected from reverse transcription amplification and amplification. As is demonstrated in the examples, such heating step may reduce the performance of the prepared biological sample in the subsequent amplification/reverse transcription amplification reaction and is therefore preferably avoided. In particular, the method should not involve heating the biological sample in contact with the extraction composition to a temperature that would denature a comprised proteinaceous RNase inhibitor prior to subjecting at least an aliquot or all of the prepared biological sample to the enzymatic reaction selected from reverse transcription and amplification. As disclosed herein, such strong proteinaceous RNase inhibitor is particularly advantageous in order to protect the labile RNA targets from degradation during preparation of the biological sample for direct amplification based detection of the target nucleic acid. Therefore, heating steps that would denature the proteinaceous RNase inhibitor should be avoided to ensure the correct performance of the extraction composition. In particularly preferred embodiments, the admixture comprising the biological sample and the extraction composition is not heated prior to subjecting at least an aliquot or all of the prepared biological sample to the subsequent enzymatic reaction selected from reverse transcription amplification and amplification. After subjecting the prepared biological sample to the enzymatic reaction, heating steps may of course be performed and are usually performed to establish e.g. the conditions for the reverse transcription reaction and/or amplification reaction and for activating “hot start” applications.

Hence, in particular where the extraction composition/solution comprises a proteinaceous RNase inhibitor, is the incubation for providing the prepared biological sample for amplification based detection of the target nucleic acid performed at a temperature where the proteinaceous RNase inhibitor is functioning and thus is not denatured. As shown in the examples, incubation may e.g. be performed at room temperature or on ice. Heating steps are avoided during this incubation step where the biological sample is in contact with the extraction composition for preparing the biological sample for amplification. After incubation, the so prepared biological sample may then be contacted with the reagents necessary for performing the amplification reaction and the amplification reaction is performed, which in an advantageous embodiment is a reverse transcription amplification. As illustrated in the examples, for sample preparation, the biological sample may furthermore be contacted with the extraction composition that has already been contacted with the reagents necessary for performing the amplification. This is e.g. feasible when processing samples, such as saliva or gargle samples, that were in advance pretreated with a digestion solution comprising a proteolytic enzyme and a reducing agent and heating (see examples). However, during incubation for preparing the biological sample, where the biological sample is thus in contact with the extraction composition comprising the proteinaceous RNase inhibitor, heating is avoided as described above. After incubation and thus preparation of the prepared biological sample, the amplification reaction, which in embodiments is a reverse transcription reaction, can be started and accordingly, all of the prepared biological sample is subjected to the amplification reaction in which the at least one target nucleic acid is then amplified. Heating steps can then be performed during the amplification/reverse transcription amplification.

The method according to the present invention provides a rapid and simple workflow that does not require elaborate pretreatment steps. Contacting the crude biological sample with the extraction composition and shortly incubating the admixture is sufficient to provide the prepared biological sample that enables a direct amplification of the target nucleic acids without prior nucleic acid purification.

Therefore, in preferred embodiments, the method according to the present invention does not involve centrifuging the prepared biological sample prior to subjecting at least an aliquot or all of the prepared biological sample to the enzymatic reaction selected from reverse transcription amplification and amplification. Accordingly, no centrifugation steps are required prior to contacting the prepared biological sample with the components necessary for performing the reverse-transcription amplification or amplification. In particular, no centrifugation steps are required to remove components (e.g. cellular debris) from the incubated admixture comprising the biological sample and the extraction composition and which provides the prepared biological sample that is subjected to the enzymatic reaction selected from reverse transcription amplification and amplification. If desired, a brief centrifugation step may be included in order to e.g. collect liquid at the bottom of the reaction vessel, e.g. after contacting the prepared biological sample with the components necessary for performing the reverse-transcription amplification or amplification as it is also described in the examples.

Advantageously, the method according to the present invention can be performed so that it does not involve removing cellular components from the prepared biological sample prior to performing an enzymatic reaction selected from reverse transcription and amplification. As disclosed herein, the method according to the present invention furthermore does not require purifying the target nucleic acid prior to performing an enzymatic reaction selected from reverse transcription and amplification and therefore allows to omit such purification step. This significantly simplifies and streamlines the workflow.

### Performing a Heating Step Prior to Contact With the Extraction Composition

According to one embodiment, the biological sample that is contacted with the extraction composition is a pathogen heat-inactivated biological sample. As disclosed herein, the biological sample may be comprised in a medium (such as a transport medium described herein) so that the biological sample contained in medium is processed as sample and contacted with the extraction composition as disclosed herein. To process a pathogen heat-inactivated biological sample is advantageous with regard to biosafety and biosecurity because heat-inactivating pathogens potentially comprised in the biological sample reduces the infection risk during sample handling and allows to simplify processing.

According to one embodiment, the method comprises heating the biological sample in the absence of the extraction composition at an elevated temperature suitable to inactivate pathogens prior to contacting the pathogen heat-inactivated biological sample with the extraction composition. Heating for inactivating pathogens potentially comprised in the biological sample prior to contacting the heat-inactivated biological sample with the extraction composition comprises heating the biological sample at a temperature that is suitable to inactivate pathogens. Heating for inactivating pathogens prior to contacting the heat-inactivated biological sample with the extraction composition may comprise heating the biological sample to a temperature ≥50° C., ≥55° C., ≥60° C. or ≥75° C. Such heating protocols for pathogen inactivation are known in the art. Heating temperatures at the lower end usually require longer heating times for pathogen inactivation, such as virus inactivation.

In preferred embodiments of the method that comprises heating prior to contacting the heat-inactivated biological sample with the extraction composition comprises heating the biological sample to a temperature ≥ 85° C., preferably ≥ 90° C. or more preferably ≥ 95° C. Heating at such higher temperatures is advantageous as the heating period necessary to achieve pathogen inactivation can be shorter, allowing the use of short heating times for pathogen inactivation as is also demonstrated in the examples. Furthermore, the use of such higher heating temperatures for pathogen inactivation may also denature proteins comprised in the crude biological sample that could negatively affect the comprised target nucleic acids.

As is demonstrated in the examples, such heating the biological sample in the absence of the extraction solution advantageously leads to pathogen inactivation and the following addition of the extraction composition of the present invention, preferably comprising (a) a non-ionic surfactant, (b) a nuclease inhibitor, preferably a proteinaceous RNase inhibitor in case of RNA targets and (c) a reducing agent, prevents the subsequent degradation of the target nucleic acid due to inhibition of the RNases thereby providing improved results without impairing signal intensity in the subsequent amplification. As is shown in the examples, these beneficial effects are not seen with prior art heating procedures which report a decrease of the signal intensity (see prior art reported in the background).

In one embodiment, heating for inactivating pathogens potentially comprised in the biological sample prior to contacting the heat-inactivated biological sample with the extraction composition comprises heating the biological sample in the collection container used for collecting the biological sample from the donor, optionally wherein the biological sample is comprised in a medium in the collection container. In one embodiment, the collection container has not been opened after collection of the biological sample and prior to heating for inactivating pathogens potentially comprised in the biological sample. In a further embodiment, an aliquot of the crude biological sample is obtained and heated for pathogen inactivation as described herein prior to contacting the heat-inactivated biological sample with the extraction composition comprises.

After heating, the biological sample may be contacted within ≤ 2 h, ≤ 1 h, ≤ 0.5 h or ≤ 20 min with the extraction composition for sample preparation. Therefore, the heat-inactivated biological samples may be directly further processed by contacting the heated biological sample with the extraction composition, if desired. Optionally, a cooling step can be performed in-between heating and contacting the biological sample with the extraction composition.

As is demonstrated in the examples, contacting the heat-inactivated biological sample with the extraction composition may also be delayed. Therefore, the pathogen heat-inactivated biological sample may be put on hold, stored or transported prior to contacting the pathogen heat-inactivated biological sample with the extraction composition of the present invention. As is apparent from the examples, short- as well as long-term storage of the pathogen heat-inactivated biological sample prior to contact with the extraction composition is possible. Good amplification results were achieved either way as is shown in the examples. According to one embodiment, the time span between heating the biological sample for providing the pathogen heat-inactivated biological sample and contacting the obtained pathogen heat-inactivated biological sample with the extraction composition is > 2 h. In embodiments, the time-span is within a range of ≥ 2 h and ≤ 150 h, ≥ 3 h and ≤ 100 h or ≥ 4 h and ≤ 75 h. In further embodiments, the time-span is at least 12 h, at least 24 h and may be at least 2 days or at least 3 days.

As is shown in the examples, heating the biological sample in the absence of the extraction composition of the invention is furthermore advantageous when processing protein rich samples, such as saliva samples or gargle samples. According to one embodiment, the biological sample, such as a saliva or gargle sample, is heated at a temperature ≥ 85° C., preferably ≥ 90° C. or more preferably ≥ 95° C. for ≤ 30 min, such as ≤ 20 min. Preferably, heating at such temperature is ≤ 15 min, thereby ensuring a fast workflow. According to one embodiment, the biological sample, such as a saliva or gargle sample, is treated with a digestion solution comprising a proteolytic enzyme and a reducing agent, prior to contacting the digested biological sample with the extraction composition according to the present invention. Digestion with the digestion solution is preferably assisted by heating at a temperature ≥ 80° C. or ≥ 85° C. Preferably, heating is at ≥ 90° C. and more preferably ≥ 95° C. This assists the lysis/digestion of the biological sample. Furthermore, heating at such high temperature ensures that the proteolytic enzyme of the digestion solution is at least at the end of the heating step inactivated. Thus, after heat-inactivation, the proteolytic enzyme comprised in the digested sample, which is then contacted with the extraction composition according to the invention, cannot degrade proteins that are used either for sample preparation and/or in the subsequent amplification reaction/reverse transcription amplification reaction, such as the proteinaceous RNase inhibitor, polymerase and/or reverse transcriptase. Suitable reducing agents were discussed elsewhere herein and it is referred to the respective disclosure. According to one embodiment, the reducing agent is selected from Tris(carboxyethyl)phosphine (TCEP), Dithiothreitol (DTT) and beta-mercaptoethanol. According to an advantageous embodiment, the reducing agent is Tris(carboxyethyl)phosphine (TCEP). The proteolytic enzyme comprised in the digestion solution may be a protease, such as preferably proteinase K. Suitable concentrations can be determined by the skilled person following the guidance presented herein and the examples. According to one embodiment, the biological sample, such as a saliva or gargle sample, is contacted after collection with the digestion solution. Contacting the digestion solution with the biological sample may be performed at a ratio of 1:4 to 1:1, such as 1:3 to 1:2. As is demonstrated by the examples, digesting a protein rich biological sample (such as a saliva or gargle sample) with a digestion solution that comprises e.g. proteinase K and a reducing agent such as TCEP and assisted by heating, increases the sensitivity of amplification based detection of target nucleic acids.

The workflow of the present invention thus may comprise an additional step in which the biological sample is first contacted with the digestion solution and heat-inactivated. According to one embodiment, the heat-inactivated biological sample is then contacted with the extraction composition according to the invention as described above. According to an alternative embodiment, the heat-inactivated biological sample is contacted with the extraction composition and components of the amplification reaction which in preferred embodiments is a reverse transcription amplification reaction.

### The Target Nucleic Acids and Target Pathogens to Be Detected

The target nucleic acid may be selected from RNA and/or DNA. As disclosed herein, the one or more, two or more or three or more target nucleic acids may be amplified/detected in the subsequent amplification step, which preferably is a reverse transcription amplification.

According to one embodiment, the at least one target nucleic acid is a pathogen-derived nucleic acid. The pathogen may be selected from the group consisting of a virus, a bacterium, a protozoan, a viroid and a fungus. As disclosed herein, the technology of the present invention allows the detection of the presence or absence of a pathogen (including different pathogens) in the biological sample, based on the amplification based detection of at least one target nucleic acid that is derived from and thus indicative for the pathogen. According to a preferred embodiment, the pathogen is a virus. A virus may be a capsid or non-capsid virus. In one embodiment, the virus is a RNA virus.

In preferred embodiments, the at least one target nucleic acid is thus a viral nucleic acid derived from a virus, preferably a RNA virus. As is demonstrated in the examples, the technology of the invention is particularly suitable for preparing crude biological samples for amplification based detection of viral target RNA derived from n RNA virus. The at least one target nucleic acid is in advantageous embodiments derived from a coronavirus, in particular a coronavirus infectious for humans.

The virus, the presence or absence of which in the biological sample may be detected using the technology of the present invention, may be a coronavirus, in particular a human coronavirus. A human coronavirus as used herein in particular refers to a coronavirus that is infectious to a human (but e.g. may also infect other animals).

According to other embodiments, the virus is an influenza virus, such as influenza-A, influenza-B, influenza-C, influenza-D, influenza-H1N1, or influenza H3N2, a parainfluenza virus, a respiratory syncytial virus (RSV), an adenovirus, an enterovirus or a rhinovirus.

According to a preferred embodiment, the at least one target nucleic acid is derived from a severe acute respiratory syndrome-related coronavirus, preferably severe acute respiratory syndrome coronavirus 2 (SARS-CoV-2) or severe acute respiratory syndrome coronavirus (SARS-CoV or SARS-CoV-1) or middle east respiratory syndrome (MERS). In preferred embodiments, the at least one target nucleic acid is a severe acute respiratory syndrome coronavirus 2 (SARS-CoV-2)-derived nucleic acid.

Hence, the target nucleic acid is derived from a coronavirus, in particular a human coronavirus. As noted, a human coronavirus in particular refers to a coronavirus that is infectious to a human. The coronavirus may in particular be a severe acute respiratory syndrome-related coronavirus, such as severe acute respiratory syndrome coronavirus 2 (SARS-CoV-2 also referred to as COVID-19) or severe acute respiratory syndrome (SARS-CoV or SARS-CoV-1). Accordingly, in a preferred embodiment, the target nucleic acid is derived from severe acute respiratory syndrome coronavirus 2 (SARS-CoV-2). A coronavirus may also be a middle east respiratory syndrome-related coronavirus, such as middle east respiratory syndrome coronavirus (MERS-CoV). In a further embodiment, a coronavirus is a human coronavirus 229E (HCoV-229E), HKU1 (HCoV-HKU1), NL63 (HCoV-NL63), OC43 (HCoV-OC43) or B814 (HCoV-B814), human enteric coronavirus (HECV). According to a further embodiment, the coronavirus is a betacoronavirus, sarbecovirus, murine hepatitis virus, murine coronavirus, hedgehog coronavirus, pipistrellus bat coronavirus, such as HKU5, HKU4, HKU1, HKU9, or HCOV-HKU1, tylonycteris derived coronavirus, rousettus derived coronavirus, Ty-BatCoV HKU5, or rhinolophus-derived coronavirus.

As is demonstrated in the examples, the technology of the invention is particularly suitable for testing biological samples for the presence of absence of SARS-CoV-2 and provides an advantageous, rapid and simple workflow that significantly improves existing SARS-CoV-2 testing methods as well as testing methods for other RNA viruses. According to one embodiment, the one or more target nucleic acids are derived from SARS-CoV-2, optionally wherein the target nucleic acid sequences are derived from the SARS-CoV-2 genes N, N1, N2, RdRP, E and Orf1b.

For reduction of experimental effort and increase of diagnostic speed, parallel detection of multiple target nucleic acids in one amplification reaction is advantageous (multiplex detection). For instance, this may include simultaneous detection of different amplicons of the same pathogen and/or parallel detection of nucleic acids derived from different pathogens. Performing the method of the present invention for multiplex detection saves time and costs compared to prior art methods. According to one embodiment, the method thus comprises detecting at least two target nucleic acids. In embodiments, the at least two target nucleic acids are derived from at least two different pathogens. The at least two pathogens may be viruses. The at least two viruses may be selected from severe acute respiratory syndrome coronavirus 2 (SARS-CoV-2), influenza-A, influenza-B, and respiratory syncytial virus (RSV). According to one embodiment, the at least two target nucleic acids are amplified simultaneously in (B), optionally wherein the at least two viruses are selected from severe acute respiratory syndrome coronavirus 2 (SARS-CoV-2), influenza-A, influenza-B, and respiratory syncytial virus (RSV). As demonstrated in the examples, the method according to the invention allows for the simultaneous detection of target nucleic acids derived from at least two different viruses, e.g. SARS-CoV-2, influenza-A, influenza-B, and RSV independent of the transport medium. The method of the invention can be extended to any pathogen of interest using respective primers.

Furthermore, the method of the present invention allows to detect different variants of the same pathogen. It can thus be used for the genotyping of pathogen variants, such as virus variants, e.g. RNA virus variants. In embodiments, the method of the invention is used for multiplex detection of different pathogen variants, such as virus variants. For instance, during the COVID-19 pandemic various new variants of SARS-CoV-2 have evolved of which some are more contagious than the wild-type strain. Consequently, identification of the virus variant with which a person is infected is of paramount importance to take effective measures and to hinder further spreading. In contrast to time-consuming sequencing analyses, the method of the present invention provides fast detection of different virus variants. As demonstrated by the examples the workflow provided herein enables clear differentiation between SARS-CoV-2 variants using specific primer/probe combinations. Thus, the method of the present invention allows rapid genotyping and identification of different virus strains or other pathogens.

### The Biological Sample

The method according to the first aspect enables the rapid preparation of biological samples for amplification based detection of the one or more target nucleic acids without prior target nucleic acid purification. The method is thus particularly suitable for preparing crude biological samples for pathogen testing by amplification, including reverse-transcription amplification, in a rapid and robust manner.

In particular, the biological sample can be a body sample (also referred to as bodily sample) and preferably is a cell-containing sample. Known embodiments of such body samples include, but are not limited to, swab samples, smear samples, blood and blood derived samples, urine, saliva, aspirates.

The biological sample may be derived from a human and may thus be a human sample. This is particularly advantageous for diagnostic applications that rely on the amplification based detection of one or more target nucleic acids e.g. in order to identify the infection with one or more pathogens, the status of a disease and/or or other health conditions that can be determined based on the presence or absence of a target nucleic acid.

According to advantageous core embodiments, the biological sample is a respiratory specimen. The respiratory specimen may be collected from the upper or lower respiratory tract and is preferably collected from the upper respiratory tract. Such biological samples are particularly suitable for the detection of viruses, including RNA viruses, such as in particular an acute respiratory syndrome-related coronavirus. As is demonstrated in the examples, the method according to the first aspect is particularly suitable to prepare respiratory specimen samples for direct amplification based detection of contained pathogenic targets, such as RNA target nucleic acids, without prior target nucleic acid isolation.

According to one embodiment, the biological sample is an oral sample, a nasal sample, a nasopharyngeal sample, an oropharyngeal sample, a throat sample or a combination of the foregoing. In a particular embodiment, the biological sample is selected from saliva, sputum, spittle, mucus, drool, bronchoalveolar lavage, pharynx secretions, nasal secretions, nasopharyngeal secretions, salivary secretions, a swab or smear derived from mouth, nose and/or throat and a combination of the foregoing.

According to a particular embodiment, the biological sample is selected from nasopharyngeal, oropharyngeal and nasal samples, preferably selected from a nasopharyngeal, oropharyngeal or nasal swab, smear or wash/aspirate samples, more preferably selected from swab or smear samples.

According to one embodiment, the biological sample is selected from saliva, sputum and mucus.

In preferred embodiments, the biological sample is a swab sample, preferably contained in a medium, and the target nucleic acid is a viral RNA. The viral RNA may be derived from a virus selected from a severe acute respiratory syndrome-related coronavirus, preferably severe acute respiratory syndrome coronavirus 2 (SARS-CoV-2) or severe acute respiratory syndrome coronavirus (SARS-CoV or SARS-CoV-1), more preferably SARS-CoV-2. In particular, the biological sample may be a nasopharyngeal, oropharyngeal or nasal swab sample, preferably contained in a medium, and the target nucleic acid is a viral RNA derived from a coronavirus, preferably a human coronavirus, such as in particular severe acute respiratory syndrome coronavirus 2 (SARS-CoV-2). In a further embodiment, the biological sample is saliva, sputum or mucus and the target nucleic acid is a viral RNA derived from a coronavirus, preferably a human coronavirus, such as in particular severe acute respiratory syndrome coronavirus 2 (SARS-CoV-2).

As shown in the examples, the biological sample may be an oral and/or throat sample. It may be selected from saliva and gargle sample. According to one embodiment, the biological sample is a saliva sample. According to one embodiment, the saliva sample is a swab soaked with saliva. For example, the biological sample may be a lollipop swab soaked with saliva, also known as “lollipop test”.

### The Medium Comprising the Biological Sample

As disclosed herein, many biological samples are collected into a medium or are transferred to a medium prior to processing the biological sample for amplification based detection of one or more target nucleic acids. It is a particular advantage of the method of the present invention that it allows to prepare a biological sample contained in a medium, such as a transport medium, for amplification based detection of at least one target nucleic acid without prior target nucleic acid purification or removal of the medium. As is demonstrated in the examples, the method according to the invention is robust and advantageously allows to prepare biological samples contained in various different media for amplification based detection of at least one target nucleic acid without prior target nucleic acid purification. The biological sample contained in medium can be directly processed and there is no need to remove the medium in advance.

In a core embodiment of the method according to the present invention, the processed biological sample is thus comprised in a medium. As disclosed herein, at least an aliquot of the medium containing the biological sample is contacted as biological sample with the extraction composition. All disclosures and embodiments described in this application for the biological sample in general, specifically apply and particularly refer to the core embodiment wherein a biological sample comprised in medium is processed and contacted with the extraction composition, even if not explicitly stated.

In certain embodiments, the biological sample is collected from the subject and directly transferred into a medium, such as a transport medium. E.g. a biological sample may be collected by a swabbing and the swab is placed into a transport medium prior to transportation and/or storage. In other embodiments, the biological sample is collected from the subject and after a delay, which optionally comprises storing and/or transporting the sample, is the biological sample contacted with the medium to provide a biological sample contained in medium that is then contacted with the extraction composition of the present invention. E.g. the biological sample may be collected into a container without any liquid and transported. Such “dry” collection of a biological sample, such as a swab sample, is sometimes performed in the situation of a pandemic where a large number of samples are collected and there is a shortage of transport media. In this case, the biological sample is preferably contacted with a liquid medium, such as physiological salt solution, to receive the biological sample in a medium. At least an aliquot of the medium containing the swabbed biological sample is then contacted as biological sample with the extraction composition according to the present invention.

According to one embodiment, the medium containing the biological sample is a transport medium. Preferably, the medium is a transport medium for swab and/or smear samples. Suitable embodiments of such transport media are known to the skilled person and furthermore described herein.

The medium is preferably an aqueous solution. The medium may be a saline solution suitable to keep the osmotic pressure in cells comprised in the biological sample when the medium is in contact with the biological sample. The medium may stabilize cells and/or viral particles comprised in the biological sample. This supports the protection of the target nucleic acids by inhibiting e.g. the release of nucleases from cells contained in the biological sample and preserving viral particles that contain the target nucleic acids. Using such media for receiving the biological sample is advantageous as it preserves the targets during transportation/storage as is well-known known in the art. The medium may also stabilize the at least one target nucleic acid against degradation. It is preferred that the medium for receiving the biological sample does not result in cross-linking or other fixation of the contained nucleic acids that could hamper and thus impair the subsequent direct amplification based detection of the one or more target nucleic acids in the prepared biological sample due to the cross-links/fixation.

According to one embodiment, the medium that comprises the biological sample is a salt containing solution. The medium is in embodiments a salt containing solution. The total salt concentration in the medium may lie in a range of 50 mM to 250 mM, such as 75 mM to 225 mM or 100 mM to 200 mM. The total salt concentration in the medium may lie in a range of 120 mM to 175 mM or 125 mM to 150 mM. Many common media used for the collection of biological samples, such as swab samples, have a salt concentration in the aforementioned range. Many common transport media used for collecting biological samples such as swab samples comprise Hank’s balanced salt solution as core component. In embodiments of the present invention, the medium in which the biological sample is contained prior to contact with the extraction composition comprises or consists of Hank’s balanced salt solution, Universal Transport Medium (UTM), Viral Transport Medium (VTM) or a medium having a total salt concentration in a range +/- 30% or +/- 20% compared to one or more of the aforementioned media. In embodiments, the medium is a physiological salt solution. The medium comprising the biological sample may be a 0.7% to 1.0% (w/v) alkali metal salt solution. In embodiments, the medium is a 0.9% (w/v) sodium chloride solution. In further embodiments, the medium comprising the biological sample is provided by a phosphate buffer, optionally a PBS buffer. As is demonstrated in the examples, the method according to the present invention allows to prepare a biological sample that is contained in such media for amplification based detection of one or more target nucleic acids without prior nucleic acid purification or removal of the medium by contacting the biological sample comprised in medium with the extraction composition according to the present invention thereby providing an admixture that comprises the extraction composition, the medium and the biological sample. This is highly advantageous, because a robust preparation method is provided that can process biological samples contained in various different media, in particular different media commonly used for receiving, e.g. collecting, respiratory specimens. Where the biological sample is comprised in a medium that contains a high amount of salt, the ionic strength of the amplification reaction buffer that is used for setting up the amplification reaction admixture may be reduced to thereby compensate the introduction of ions into the amplification reaction admixture due to the prepared biological sample that comprises the extraction composition, the biological sample and the salt-containing medium. This embodiment allows to incorporate a high amount of prepared biological sample into the amplification reaction admixture (e.g. up to 40%, up to 50% or up to 60% of the total volume of the amplification reaction admixture that comprises all components used in the amplification, which preferably is a reverse transcription amplification) without detrimental inhibition of the amplification reaction by the components that are carried over from the salt-containing medium into the prepared biological sample and thus the amplification reaction. Alternatively, the amount of prepared biological sample in the amplification reaction admixture can be reduced to ensure a high performance of the amplification reaction, in particular a reverse transcription amplification reaction.

According to one embodiment, the method comprises
obtaining the biological sample contained in medium and optionally agitating the sample;
contacting an aliquot of the obtained biological sample contained in medium with the extraction composition thereby providing an admixture comprising the extraction composition, the biological sample and the medium; and
incubating the admixture to provide the prepared biological sample for amplification based detection of the at least one target nucleic acid without prior target nucleic acid purification.

### Further Use of the Prepared Biological Sample

The method according to the first aspect provides a prepared biological sample for amplification based detection of one or more target nucleic acids without prior target nucleic acid purification. After incubation of the admixture comprising the biological sample, the extraction composition and optionally the medium that contained the biological sample to provide the prepared biological sample, the method according to the first aspect preferably further comprises
subjecting at least an aliquot of the prepared biological sample to an enzymatic reaction, preferably a reverse transcription and/or amplification reaction, and performing the enzymatic reaction.

As disclosed herein, a reverse transcription reaction and/or an amplification reaction, such as a reverse-transcription and amplification reaction, can be performed using the prepared biological sample. As is demonstrated by the examples, using an extraction composition as disclosed herein provides a prepared biological sample that is suitable for amplification based detection of one or more target nucleic acids, such as RNA target nucleic acids while ensuring a good performance and sensitivity.

According to one embodiment, the performed enzymatic reaction comprises an amplification reaction. Any kind of amplification can be performed, including but not limited to (i) a reverse transcription amplification reaction; (ii) a reverse transcription PCR; (iii) an isothermal amplification reaction; (iv) a polymerase chain reaction (PCR); (v) a quantitative PCR; (vi) a quantitative reverse transcription PCR or (vii) a digital PCR. As used herein, the term “PCR” comprises PCR (polymerase chain reaction; DNA amplification) as well as RT-PCR (reverse transcription - polymerase chain reaction; RNA amplification). In particular preferred embodiments, the PCR is a semi-quantitative or more preferably a quantitative PCR, such as a quantitative reverse-transcription PCR. Performing a quantitative PCR is particularly preferred for pathogen testing.

According to a preferred embodiment, the enzymatic reaction is a reverse-transcription amplification reaction, preferably a quantitative reverse-transcription polymerase chain reaction. As is demonstrated in the examples, the method according to the present invention is particularly useful in order to amplify RNA target nucleic acids, which is a core application for pathogen testing, e.g. in order to detect the presence of absence of SARS-CoV-2 and other RNA viruses.

Advantageously, in all methods according to the present invention the steps of
contacting the biological sample with the extraction composition,
incubating the admixture comprising the biological sample in contact with the extraction composition, and
performing the enzymatic reaction, preferably a reverse-transcription amplification reaction or DNA amplification reaction,
are performed within the same reaction vessel.

To perform all steps in a single vessel saves time and consumables and therefore represents an improvement compared to state of the art technologies in which the nucleic acid extraction and amplification steps are performed in separate vessels. For instance, the above described steps can be performed consecutively within one PCR tube or one well of a PCR plate. In times with high-throughput demand, as it is e.g. the case in pandemic situation, such efficient and accurate pathogen detection method as it is provided by the present invention that saves time and consumables is particularly advantageous.

According to one embodiment, the prepared biological sample that is subjected to the enzymatic reaction provides at least 20%, at least 30%, at least 40% or at least 45% of the total reaction volume of the enzymatic reaction. In embodiments, the prepared biological sample that is subjected to the enzymatic reaction provides up to 60% or up to 50% of the total reaction volume of the enzymatic reaction. Therefore, in preferred embodiments, wherein an amplification reaction, such as the reverse transcription amplification reaction, is performed, the prepared biological sample provides at least 20%, at least 30%, at least 40% or at least 45% of the total volume of the amplification reaction admixture which comprises the prepared biological sample and all components necessary for performing the amplification. In embodiments, the prepared biological sample provides up to 60% or up to 50% of the total volume of the amplification reaction admixture which comprises the prepared biological sample and all components necessary for performing the amplification as is also demonstrated in the examples. The possibility to subject a high volume of the prepared biological sample to the amplification reaction, such as the reverse transcription amplification reaction is advantageous because it increases the sensitivity. As disclosed herein and shown in the examples, despite processing a crude biological sample without prior nucleic acid purification, the pretreatment step disclosed herein wherein the crude biological sample is contacted with the extraction composition provides prepared biological samples in which the target nucleic acids, including RNA target nucleic acids can be reliably identified with good sensitivity. The components of the extraction solution do not interfere with the subsequent amplification or reverse transcription amplification and can furthermore balance differences in the biological samples thereby ensuring robust results.

Preferred embodiments of the method according to the first aspect are again described in the following.

According to one embodiment, the method is for preparing a biological sample for amplification based detection of at least one pathogenic RNA target nucleic acid comprised in the biological sample without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture,
   optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid; and
subjecting at least an aliquot or all of the prepared biological sample to a reverse transcription and amplification reaction and performing the reaction.

As disclosed herein, the performed amplification allows detecting the presence or absence of the one more target nucleic acids in the biological sample. This is advantageous e.g. for pathogen testing as disclosed herein. Depending on whether an amplification signal is obtained for the one or more pathogen derived target nucleic acids in the amplification reaction, it can be determined whether the biological sample is positive or negative for the target pathogen(s). As shown by the examples, multiplex detections are feasible. E.g. two or more target nucleic acids derived from at least two different pathogens, such as different viruses, can be detected. Furthermore, the at least two target nucleic acids may be derived from two or more different variants of the same pathogen, such as virus variants.

According to one embodiment, the method is for preparing a biological sample for amplification based detection of at least one pathogenic RNA target nucleic acid without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture, wherein the admixture comprises
   (i) the non-ionic surfactant originating from the extraction solution in a concentration that lies in a range of 0.1% to 10% (w/v), optionally 0.2% to 5% (w/v) or 0.3% to 3% (w/v), and
   (ii) the reducing agent originating from the extraction solution in a concentration that lies in a range of 0.1 mM to 15 mM, optionally 0.2 mM to 10 mM, 0.3 mM to 5 mM or 0.4 mM to 1.5 mM;
   optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid; and
subjecting at least an aliquot or all of the prepared biological sample to a reverse transcription and amplification reaction and performing the reaction.

According to one embodiment, the method is for preparing a biological sample for amplification based detection of at least one pathogenic RNA target nucleic acid comprised in the biological sample without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture; optionally wherein the method comprises heating the biological sample in the absence of the extraction solution to inactivate pathogens potentially comprised in the biological sampleprior to contacting at least an aliquot of the pathogen heat-inactivated biological sample with the extraction solution;
incubating the admixture to provide the prepared biological sample for amplification based detection of the at least one pathogenic RNA target nucleic acid; and
subjecting at least an aliquot or all of the prepared biological sample to a reverse transcription and amplification reaction and performing the reaction, wherein at least the steps of
   contacting the biological sample with the extraction solution to prepare the admixture,
   incubating the admixture, and
   performing the reverse-transcription amplification reaction,
are performed within the same reaction vessel.

According to one embodiment, the method is for preparing a biological sample for amplification based detection of at least one pathogenic RNA target nucleic acid without prior nucleic acid purification, comprising
contacting the biological sample contained in medium with an extraction solution comprising
   (a) at least one non-ionic surfactant,
   (b) at least one proteinaceous RNase inhibitor, and
   (c) at least one reducing agent,
   to prepare an admixture, wherein the admixture comprises
   (i) the non-ionic surfactant originating from the extraction solution in a concentration that lies in a range of 0.1% to 10% (w/v), optionally 0.2% to 5% (w/v) or 0.3% to 3% (w/v), and
   (ii) the reducing agent originating from the extraction solution in a concentration that lies in a range of 0.1 mM to 15 mM, optionally 0.2 mM to 10 mM, 0.3 mM to 5 mM or 0.4 mM to 1.5 mM;
   optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture for at least 1 min, preferably at least 1.5 min or at least 2 min, to provide the prepared biological sample for amplification based detection of the at least one pathogenic RNA target nucleic acid; and
subjecting at least an aliquot or all of the prepared biological sample to a reverse transcription and amplification reaction and performing the reaction;
   wherein the prepared biological sample that is subjected to the reverse transcription and amplification reaction provides at least 30%, at least 40% or at least 45% of the total reaction volume of the reverse transcription and amplification reaction, and
   wherein at least the steps of
      contacting the biological sample with the extraction solution to prepare the admixture,
      incubating the admixture, and
      performing the reverse-transcription amplification reaction,
are performed within the same reaction vessel.

According to one embodiment, the method is for preparing a respiratory biological sample for amplification based detection of at least one pathogenic RNA target nucleic acid comprised in the biological sample without prior nucleic acid purification, comprising
contacting the respiratory biological sample contained in medium with an extraction solution comprising
   (a) at least one non-ionic surfactant,
   (b) at least one proteinaceous RNase inhibitor, and
   (c) at least one reducing agent,
   to prepare an admixture,
   optionally wherein the method comprises heating the respiratory biological sample contained in medium in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid; and
subjecting at least an aliquot or all of the prepared biological sample to a reverse transcription and amplification reaction and performing the reaction for detecting the presence or absence of the at least one pathogenic RNA target nucleic acid;
   wherein the prepared biological sample that is subjected to the reverse transcription and amplification reaction provides at least 30% or at least 40% of the total reaction volume of the reverse transcription and amplification reaction, which preferably is a quantitative RT-PCR reaction, and
   wherein at least the steps of
      contacting the respiratory biological sample contained in medium with the extraction solution to prepare the admixture,
      incubating the admixture, and
      performing the reverse-transcription amplification reaction,
      are performed within the same reaction vessel, and
   wherein the reverse transcription and amplification reaction comprises primers suitable for amplifying one or more, preferably two or more, target nucleic acids derived from a severe acute respiratory syndrome-related coronavirus, preferably severe acute respiratory syndrome coronavirus 2 (SARS-CoV-2).

According to one embodiment, the method is for preparing a biological sample for amplification based detection of at least one RNA target nucleic acid, such as at least one pathogenic RNA target nucleic acid, comprised in the biological sample without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture,
   wherein the method comprises heating the biological sample in the absence of the extraction solution to inactivate pathogens potentially comprised in the biological sample prior to contacting at least an aliquot of the pathogen heat-inactivated biological sample with the extraction solution, wherein the biological sample is heated to a temperature ≥ 85° C., preferably ≥ 90° C., more preferably ≥ 95° C.;
incubating the admixture to provide the prepared biological sample for amplification based detection of the at least one RNA target nucleic acid.

As shown in the examples, this embodiment is particularly suitable for protein-rich samples such as saliva or gargle samples. According to one embodiment, the method is for preparing a biological sample, such as a saliva or gargle sample, for amplification based detection of at least one RNA target nucleic acid, such as at least one pathogenic RNA target nucleic acid, comprised in the biological sample without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture,
   wherein the method comprises heating the biological sample in the absence of the extraction solution to a temperature ≥ 85° C., preferably ≥ 90° C., more preferably ≥ 95° C. prior to contacting at least an aliquot of the heat-treated biological sample with the extraction solution,
   optionally wherein heating occurs in the presence of a digestion solution that has been contacted with the biological sample, wherein the digestion solution comprises a proteolytic enzyme and a reducing agent,
incubating the admixture to provide the prepared biological sample for amplification based detection of the at least one RNA target nucleic acid.

After incubation, a reverse transcription and amplification reaction can be performed to detect the presence or absence of the RNA target nucleic acid in the biological sample. In one embodiment, a reverse transcription amplification is performed which provides a fast workflow. The examples illustrate various embodiments how the prepared biological sample can be contacted with the necessary reagents for performing the amplification reaction, respectively reverse transcription amplification reaction. E.g. the reagents necessary for reverse transcription/amplification can be added to the prepared biological sample or vice versa. Furthermore, the reagents necessary for amplification may also be pre-mixed with or added at the same time as the extraction composition according to the invention. As described elsewhere herein, the amplification/reverse transcription amplification reaction can then directly be initiated after the prepared biological sample has been provided by incubation in contact with the extraction composition (which is performed in the absence of a heating step as described elsewhere herein). The reaction can directly start, if desired, because the reagents necessary for amplification/reverse transcription amplification are already contained in the admixture. Also mixed embodiments are feasible, wherein some but not all, reagents necessary for the amplification/reverse transcription amplification are added after the prepared sample was provided by incubating the (preferably predigested) biological sample in the presence of the extraction composition. As is shown in the examples, this embodiment that is based on a pre-digestion using a digestion solution comprising a proteolytic enzyme (e.g. proteinase K) and a reducing agent (e.g. TCEP) assisted by heating at a high temperature as indicated above is e.g. advantageous for processing protein-rich sample types, such as saliva and gargle samples.

### THE METHODS ACCORDING TO THE SECOND AND THIRD ASPECT

According to a second aspect, a method is provided for amplification based detection of at least one target nucleic acid comprised in a biological sample without prior purification of the target nucleic acid, comprising
(A) preparing the biological sample for amplification based detection of the target nucleic acid, wherein preparing comprises
   contacting the biological sample with an extraction composition comprising
      (a) at least one surfactant,
      (b) at least one nuclease inhibitor, and
      (c) optionally at least one reducing agent, and
   incubating the admixture comprising the biological sample in contact with the extraction composition to provide the prepared biological sample;
(B) subjecting at least an aliquot or all of the prepared biological sample to an amplification reaction and amplifying the at least one target nucleic acid, optionally wherein a reverse transcription reaction is performed in order to reverse transcribe RNA to cDNA prior to amplification. As disclosed herein, the at least one target nucleic acid is in core embodiments derived from a pathogen.

According to a third aspect, a method is provided for detecting the presence or absence of a pathogen in a biological sample based on amplifying at least one target nucleic acid derived from the pathogen, comprising
(A) preparing the biological sample for amplification based detection of the target nucleic acid, wherein preparing comprises
   contacting the biological sample with an extraction composition comprising
      (a) at least one surfactant,
      (b) at least one nuclease inhibitor, and
      (c) optionally at least one reducing agent, and
   incubating the admixture comprising the biological sample in contact with the extraction composition to provide the prepared biological sample;
(B) subjecting at least an aliquot or all of the prepared biological sample to an amplification reaction and amplifying the at least one target nucleic acid, optionally wherein a reverse transcription reaction is performed in order to reverse transcribe RNA to cDNA prior to amplification.

In the methods according to the second and third aspect, (A) preparing the biological sample for amplification based detection of the target nucleic acid is preferably performed as described above in conjunction with the method according to the first aspect. Therein, suitable and preferred embodiments of the extraction composition of the present invention, which preferably is an extraction solution, are described in detail and it is referred to the respective disclosure which also applies here.

Pathogens that can advantageously be detected using the methods of the present invention were already disclosed in the context of the method according to the first aspect of the present invention and it is referred to the respective disclosure. As disclosed, the pathogen may be a virus, a bacterium, a protozoan, a viroid or a fungus. According to a preferred embodiment, the pathogen is a virus. A virus may be a capsid or non-capsid virus. In one embodiment, the virus is a RNA virus. As is demonstrated in the examples, the technology of the invention is particularly suitable for preparing crude biological samples for amplification based detection of viral target RNA derived from a RNA virus. The at least one target nucleic acid is in advantageous embodiments derived from a coronavirus, in particular a coronavirus infectious for humans. Hence, the pathogen to be detected may be a human coronavirus. As noted, a human coronavirus in particular refers to a coronavirus that is infectious to a human. The coronavirus to be detected may be a severe acute respiratory syndrome-related coronavirus, such as severe acute respiratory syndrome coronavirus 2 (SARS-CoV-2 also referred to as COVID-19) or severe acute respiratory syndrome (SARS-CoV or SARS-CoV-1). A coronavirus may also be a middle east respiratory syndrome-related coronavirus, such as middle east respiratory syndrome coronavirus (MERS-CoV). In a further embodiment, a coronavirus is a human coronavirus 229E (HCoV-229E), HKU1 (HCoV-HKU1), NL63 (HCoV-NL63), OC43 (HCoV-OC43) or B814 (HCoV-B814), human enteric coronavirus (HECV).

According to a further embodiment, the coronavirus is a betacoronavirus, sarbecovirus, murine hepatitis virus, murine coronavirus, hedgehog coronavirus, pipistrellus bat coronavirus, such as HKU5, HKU4, HKU1, HKU9, or HCOV-HKU1, tylonycteris derived coronavirus, rousettus derived coronavirus, Ty-BatCoV HKU5, or rhinolophus-derived coronavirus. In a core embodiment, embodiment the pathogen to be detected is severe acute respiratory syndrome coronavirus 2 (SARS-CoV-2).

Suitable embodiments for the at least one target nucleic acid are also disclosed in the context of the method according to the first aspect and it is referred to the above disclosure which also applies here. As disclosed therein, preferably, the one or more target nucleic acids are RNA targets and the amplification reaction is a reverse transcription amplification reaction. This embodiment is particular advantageous for detecting the presence or absence of a RNA virus, such as a coronavirus in the biological sample. According to a preferred embodiment, one or more SARS-CoV-2 target nucleic acids are reverse transcribed and amplified for detecting the presence of absence of SARS-CoV-2 in the biological sample. As disclosed herein, the one or more target nucleic acid sequences may be derived from the SARS-CoV-2 genes N, N1, N2, RdRP, E and Orf1b. Suitable primers and furthermore probes that can be used in a reverse-transcription amplification reaction, preferably a quantitative RT-PCR are known in the art and e.g. published by the CDC and WHO.

Embodiments of the biological sample are disclosed in conjunction with the method according to the first aspect and it is referred to the corresponding disclosure which also applies here. As discussed, in a preferred embodiment, that is particularly suitable for RNA virus such as coronavirus testing, the biological sample is a respiratory specimen comprised in a medium. Common media used for receiving biological samples, in particular respiratory specimens such as nasopharyngeal, oropharyngeal and nasal samples, such as in particular nasopharyngeal, oropharyngeal or nasal swab or smear samples, are disclosed in conjunction with the method according to the first aspect and it is referred to the respective disclosure.

According to one embodiment, the method according to the second and/or third aspect is characterized in that preparing in (A) comprises
obtaining the biological sample contained in medium and optionally agitating the sample;
contacting at least an aliquot of the obtained biological sample contained in medium with the extraction composition thereby providing an admixture; and
incubating the admixture to provide the prepared biological sample for amplification based detection of the target nucleic acid.

In embodiments, the method according to the second and/or third aspect comprises heating the biological sample in the absence of the extraction composition at a temperature suitable to inactivate pathogens prior to contacting the pathogen heat-inactivated biological sample with the extraction composition. Further details and advantages of such heating step are disclosed in the context of the method according to the first aspect and it is referred to the respective disclosure which also applies here. The pathogen heat-inactivated biological sample may be further processed as disclosed in conjunction with the method according to the first aspect.

Further embodiments, wherein such heating step in the absence of the extraction composition is performed after contacting the biological sample with a digestion solution comprising a proteolytic enzyme (preferably a protease such as proteinase K) and a reducing agent (such as TCEP) were described above and are also illustrated in the examples. Such workflows wherein the biological sample is contacted with the digestion solution comprising the proteolytic enzyme and the reducing agent for digestion of the biological sample assisted by heating (preferably at a temperature ≥ 80° C., such as ≥ 85° C., preferably ≥ 90° C., more preferably ≥ 95° C. - see above) are particularly suitable for processing protein-rich samples, such as saliva and gargle samples. As demonstrated by the examples, the sensitivity can be improved.

As disclosed herein, the prepared biological sample that is subjected to the amplification reaction may provide at least 20%, at least 30%, at least 40% or at least 45% of the total reaction volume of the amplification reaction. In embodiments, the prepared biological sample that is subjected to the amplification reaction provides up to 60% or up to 50% of the total reaction volume of the amplification reaction. Furthermore, at least the steps of
contacting the biological sample with the extraction solution to prepare the admixture,
incubating the admixture, and
performing the reverse-transcription amplification reaction,
may be performed within the same reaction vessel. As disclosed in conjunction with the method according to the first aspect, this is particularly advantageous as it is rapid and saves consumables.

According to a preferred embodiment of the method according to the first and/or second aspect, the biological sample is a respiratory biological sample contained in medium and the method is for amplification based detection of at least one pathogenic RNA target nucleic acid comprised in the biological sample without prior nucleic acid purification,
wherein the extraction composition used in (A) is an extraction solution comprising (a) a non-ionic surfactant, (b) a proteinaceous RNase inhibitor, and (c) a reducing agent, optionally wherein the extraction solution consists essentially of or consists of the aforementioned active ingredients comprised in liquid;
wherein in (B) a reverse transcription and amplification reaction, preferably a quantitative reverse transcription PCR, is performed for detecting the presence or absence of the at least one pathogenic RNA target nucleic acid;
   wherein the prepared biological sample that is subjected to the reverse transcription and amplification reaction provides at least 25%, at least 30% or at least 40% of the total reaction volume of the reverse transcription and amplification reaction, and
   wherein at least the steps of
      contacting the respiratory biological sample contained in medium with the extraction solution to prepare the admixture,
      incubating the admixture comprising the extraction solution, the biological sample, and medium to provide the prepared biological sample, and
      performing the reverse-transcription amplification reaction,
   are performed within the same reaction vessel, and
   wherein the reverse transcription and amplification reaction comprises primers suitable for amplifying one or more, preferably two or more, target nucleic acids derived from a severe acute respiratory syndrome-related coronavirus, preferably severe acute respiratory syndrome coronavirus 2 (SARS-CoV-2).

Further details of (A) and (B) are also disclosed in conjunction with the method according to the first aspect and it is referred to the respective disclosure.

As disclosed herein, it is a particular advantage that the method according to the present invention allows the processing of crude biological samples comprised in a salt containing medium. Examples of such media are disclosed above. Where the medium in which the biological sample is contained contains a high amount of salt, the ionic strength of the amplification reaction buffer that is used for setting up the amplification reaction admixture is preferably reduced to thereby compensate the introduction of ions into the amplification reaction admixture due to the prepared biological sample that comprises the extraction composition, the biological sample and the salt-containing medium. As is shown in the examples, if the overall chloride concentration is too high, this can inhibit e.g. the reverse transcription reaction. Furthermore, other ions such as sodium may inhibit the DNA polymerase which in one embodiment is a Taq polymerase. Therefore, to compensate these effects it is advantageous to reduce the ionic strength and in particular the chloride concentration in the amplification reaction buffer.

According to one embodiment, subjecting at least an aliquot or all of the prepared biological sample to an amplification reaction in (B) comprises contacting the prepared biological sample with the components used for performing the amplification or reverse transcription amplification reaction thereby providing an amplification reaction admixture.

Furthermore, as shown in the examples, components/reagents necessary for performing the amplification reaction or reverse transcription amplification reaction may also be pre-mixed with the extraction composition. Such embodiment is e.g. feasible in conjunction with the workflow that predigests the biological sample using a digestion solution and heating prior to contacting the biological sample with the extraction composition of the invention.

In one embodiment, the prepared amplification reaction admixture comprises:
(a) the prepared biological sample;
(b) a DNA polymerase;
(c) optionally a reverse transcriptase, which is included in case a reverse transcription amplification is performed;
(d) an amplification reaction buffer comprising a Mg²⁺ source, a buffering agent and optionally further additives;
(e) nucleotides, preferably a dNTP mix, optionally wherein the nucleotides comprise modified nucleotides or dUTP; and
(f) primers for amplifying the one or more target nucleic acids and optionally probes.

In preferred embodiments, the method comprises contacting the prepared biological sample with an amplification master mix comprising components (b) to (e) and separately provided primers for amplifying the one or more target nucleic acids (and optionally probes which is advantageous in case a quantitative amplification is performed).

According to an advantageous embodiment, the ionic strength of the amplification reaction buffer (d) or the amplification master mix comprising components (b) to (e) is reduced to thereby compensate the introduction of ions, in particular ions derived from alkali metal salts and/or chlorides, into the amplification reaction admixture due to the prepared biological sample that may contain the medium. As is demonstrated in the examples, many commonly used media comprise a high concentration of salts that may impair the amplification reaction, in particular a reverse transcription amplification reaction. Therefore, this embodiment is advantageous to compensate the ions introduced by the medium thereby ensuring that the amplification can work properly enabling sensitive testing.

According to one embodiment, the amplification reaction buffer (d) has one or more, preferably two or more, more preferably three of more of the following characteristics: (aa)The amplification reaction buffer (d) does not comprise sodium chloride in a concentration ≥ 30 mM. In embodiments, it does not comprise sodium chloride in a concentration ≥ 20 mM, ≥ 15 mM, ≥ 10 mM or ≥ 5 mM. Preferably, the amplification reaction buffer (d) contains no sodium chloride.

(bb) The amplification reaction buffer (d) does not comprise potassium chloride in a concentration ≥ 30 mM, such as ≥ 20 mM, ≥ 15 mM, ≥ 10 mM or ≥ 5 mM. Preferably, the amplification reaction buffer (d) contains no potassium chloride.

(cc) In preferred embodiments, the amplification reaction buffer (d) does not comprise potassium chloride or sodium chloride.

(dd) The alkali metal chloride concentration in the amplification reaction buffer (d) is ≤ 30 mM, ≤ 20 mM, ≤ 15 mM or ≤ 10 mM. Preferably, the amplification reaction buffer (d) does not contain alkali metal chlorides.

(ee) The alkali metal salt concentration in the amplification reaction buffer (d) is ≤ 30 mM, such as ≤ 20 mM, ≤ 15 mM or ≤ 10 mM. Preferably, the amplification reaction buffer (d) does not contain alkali metal salts.

In advantageous embodiments, the amplification reaction buffer (d) comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)-propanesulphonic acid, N-(2-hydroxyethyl)piperazine-N′-(2-ethanesulphonic acid), piperazine-1,4-bis(2-ethanesulphonic acid), N-cyclohexyl-2-aminoethanesulphonic acid and 2-(N-morpholino)ethanesulphonic acid. Preferably, the buffering agent is selected from tris(hydroxymethyl)aminomethane and 3-(N-morpholino)propanesulphonic acid.

In embodiments, the pH of the amplification reaction buffer (d) is adjusted with an acid that does not comprise chloride. As is demonstrated in the examples, the pH may be adjusted using an organic acid, preferably a carboxylic acid. This further reduces the chloride burden and therefore provides a robust method for processing different types of prepared biological samples. As disclosed herein, it allows the processing of crude biological samples that are comprised in salt-containing media or other media of high ionic strength that are prepared using the extraction composition according to the present invention.

According to one embodiment, the amplification reaction buffer (d) is characterized in that:
(i) it does not comprise potassium chloride or sodium chloride;
(ii) it comprises a buffering agent that does not comprise chloride ions, optionally wherein the buffering agent is selected from tris(hydroxymethyl)aminomethane and 3-(N-morpholino)-propanesulphonic acid, and
(iii) the pH of the amplification reaction buffer (d) is adjusted with an organic acid, preferably a carboxylic acid.

According to one embodiment, the amplification master mix comprising components (b) to (e) has one or more, preferably two or more or more preferably three or more of the following characteristics:

(aa) It does not comprise sodium chloride in a concentration ≥ 50 mM, ≥ 20 mM, ≥ 15 mM or ≥ 10 mM. Preferably, it contains no sodium chloride.

(bb) It does not comprise potassium chloride in a concentration ≥ 100 mM, ≥ 75 mM, ≥ 60 mM or ≥ 50 mM. Optionally, it contains no potassium chloride. However, a small amount of potassium chloride may be comprised in the amplification master mix, as it might be introduced via the comprised enzyme(s) such as the DNA polymerase. However, as disclosed herein, preferably no potassium chloride is introduced via the amplification reaction buffer (d).

(cc) The alkali metal chloride concentration in the amplification master mix is ≤ 100 mM, ≤ 75 mM, ≤ 60 mM, ≤ 50 mM or ≤ 45 mM. As disclosed herein, it may also not contain alkali metal chlorides.

(dd) The alkali metal salt concentration in the amplification master mix is ≤ 100 mM, ≤ 75 mM, such as ≤ 60 mM, ≤ 50 mM or ≤ 45 mM.

(ff) The chloride ion concentration is ≤ 250 mM, preferably ≤ 200 mM, ≤ 175 mM or ≤ 150 mM. As disclosed in the examples, the amplification master mix may be provided in concentrated form and the above mentioned concentrations are in particular suitable for a 3x or 4x amplification master mix.

The amplification master mix comprising components (b) to (e) may have one or both of the following characteristics:
(aa) it comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)propanesulphonic acid, N-(2-hydroxyethyl)piperazine-N′-(2-ethanesulphonic acid), piperazine-1,4-bis(2-ethanesulphonic acid), N-cyclohexyl-2-aminoethanesulphonic acid and 2-(N-morpholino)ethanesulphonic acid and preferably is selected from tris(hydroxymethyl)aminomethane and 3-(N-morpholino)propanesulphonic acid;
(bb) the pH is adjusted with an organic acid, preferably a carboxylic acid, optionally wherein the carboxylic acid selected from acetic acid, formic acid, propionic acid and butyric acid, preferably acetic acid.

These embodiments that use accordingly optimized amplification reagents allow to incorporate a high amount of prepared biological sample into the amplification reaction admixture (e.g. up to 40%, up to 50% or up to 60% of the total volume of the amplification reaction admixture that comprises all components used in the amplification, which preferably is a reverse transcription amplification) without detrimental inhibition of the amplification reaction by the components that are carried over from the salt-containing medium into the prepared biological sample and thus the amplification reaction. Alternatively, the amount of prepared biological sample in the amplification reaction admixture can be reduced to ensure a high performance of the amplification reaction, in particular a reverse transcription amplification reaction.

### THE KIT ACCORDING TO THE FOURTH ASPECT

According to a fourth aspect, a kit for performing the method according to the first, second and/or third aspect is provided. Said kit comprises:

(a) an extraction composition according to the present invention.

As disclosed, the extraction composition according to the present invention comprises
(a) at least one surfactant,
(b) at least one nuclease inhibitor, and
(c) optionally at least one reducing agent.
The extraction composition according to the present invention, including suitable and preferred embodiments for (a) the surfactant, (b) the at least one nuclease inhibitor which preferably is an RNase inhibitor and (c) the reducing agent as well as suitable concentration ranges are described in detail in conjunction with the method according to the first aspect and it is referred to the respective disclosure which also applies here. The reducing agent is preferably comprised in the extraction composition which preferably is an extraction solution.

According to one embodiment, the extraction solution comprises
(a) at least one non-ionic surfactant,
(b) at least one proteinaceous RNase inhibitor, and
(c) at least one reducing agent.

Suitable and preferred embodiments of the non-ionic surfactant and the reducing agent as well as suitable concentrations were described above and it is referred to the respective disclosure. As disclosed herein, the extraction solution may consist essentially of or may consist of the aforementioned active ingredients (a) to (c) comprised in a carrier liquid.

According to one embodiment, the extraction solution comprises
(a) at least one polyoxyethylene-based non-ionic surfactant,
(b) at least one proteinaceous RNase inhibitor, and
(c) at least one reducing agent selected from Tris(carboxyethyl)phosphine (TCEP), Dithiothreitol (DTT), N-acetyl cysteine, THPP (Tris(hydroxypropyl)phosphine) and 1-thioglycerol.

As disclosed herein, the active ingredients of the extraction solution may consist essentially of or may consist of
(a) a non-ionic surfactant, preferably a polyoxyethylene-based non-ionic surfactant,
(b) a proteinaceous RNase inhibitor, and
(c) a reducing agent, preferably selected from Tris(carboxyethyl)phosphine (TCEP), Dithiothreitol (DTT), N-acetyl cysteine, THPP (Tris(hydroxypropyl)phosphine) and 1-thioglycerol.

Suitable and preferred embodiments of the one polyoxyethylene-based non-ionic surfactant as well as suitable concentrations are described above and it is referred to the respective disclosure. In advantageous embodiments, the non-ionic surfactant comprised in the extraction solution is selected from polyoxyethylene fatty acid esters, in particular polyoxyethylene sorbitan fatty acid esters, and polyoxyethylene fatty alcohol ethers.

According to a preferred embodiment, the extraction solution comprises
(a) at least one polysorbate,
(b) at least one proteinaceous RNase inhibitor, and
(c) Tris(carboxyethyl)phosphine (TCEP).

As is demonstrated by the examples, such extraction solution is very advantageous and allows to prepare even difficult biological samples, including respiratory specimens comprised in medium, for direct reverse transcription and amplification of comprised RNA target nucleic acids (such as viral RNA targets) with favorable sensitivity. Suitable polysorbates that can be included into the extraction solution as non-ionic surfactant are disclosed above and it is referred to the respective disclosure. As described, the polysorbate may be selected from polysorbate 20, polysorbate 40, polysorbate 60 and polysorbate 80. Polysorbate 20 is a particularly preferred polysorbate that can be included in the extraction solution as non-ionic surfactant. In one embodiment, the active ingredients of the extraction solution may consist essentially of or may consist of
(a) the polysorbate (e.g. polysorbate 20),
(b) the proteinaceous RNase inhibitor, and
(c) Tris(carboxyethyl)phosphine (TCEP).

According to one embodiment, the kit according to the fourth aspect comprises a digestion solution comprising a proteolytic enzyme and a reducing agent. According to one embodiment, the reducing agent is selected from Tris(carboxyethyl)phosphine (TCEP), Dithiothreitol (DTT) and beta-mercaptoethanol and preferably is Tris(carboxyethyl)phosphine (TCEP). The proteolytic enzyme comprised in the digestion solution may be a protease, such as preferably proteinase K. Suitable concentrations can be determined by the skilled person following the guidance presented herein and the examples. As is demonstrated by the examples, digesting a protein rich biological sample (such as a saliva or gargle sample) with a digestion solution that comprises e.g. proteinase K and a reducing agent such as TCEP and assisted by heating, increases the sensitivity of amplification based detection of target nucleic acids.

In advantageous embodiments, the kit according to the fourth aspect additionally comprises one or more and preferably all of the following components:
(b) a DNA polymerase;
(c) a reverse transcriptase;
(d) an amplification reaction buffer comprising a Mg²⁺ source, a buffering agent and optionally further additives;
(e) nucleotides, preferably a dNTP mix; and
(f) primers for amplifying the at least one target nucleic acid.

A reverse transcriptase is preferably included in case a reverse transcription is performed. According to one embodiment components (b) to (e) are comprised in a single composition thereby providing an amplification master mix. The amplification master mix may be provided in a concentrated form, e.g. at least 2x, at least 3 or at least 4x. Using a concentrated amplification master mix is advantageous as it allows the incorporation of a high amount of prepared biological sample into the amplification reaction admixture that comprises the prepared biological sample, the amplification master mix comprising components (b) to (e) and further separately added components that are used in the amplification reaction such as primers, and optionally probes, dyes, internal controls and the like. Such additional components, e.g. probes and/or dyes for quantitative (real-time) PCR, such as quantitative RT-PCR, internal controls and the like, may also be comprised in the kits. These standard components that are also used in prior art amplification methods are well-known to the skilled person and therefore, do not need any detailed description. The nucleotides may also comprise modified nucleotides. The nucleotides enable the amplification of the target nucleic acid. dUTP may also be included. In embodiments, component (e) is a dNTP mix comprising dATP, dCTP, dGTP, and dTTP.

In embodiments, a direct amplification master mix is provided which comprises components (b) and (d) to (f), and optionally (c). Such direct amplification master mix thus already comprises the primers (and optionally probes) required for amplifying the one or more target nucleic acids.

The kit may also comprise additional components/additives used in the amplification reaction. Such components may also be comprised in an amplification master mix.

According to one embodiment, the kit comprises one or more of the following additives which are preferably comprised in the amplification reaction buffer (d) and may thus be contained in an amplification master mix that comprises components (b) to (e):
an ammonium salt, optionally selected from ammonium sulfate and ammonium chloride;
polyethylene glycol;
N,N,N-trimethylglycine;
serum albumin;
a metal ion chelator, optionally EGTA;
glycerol;
fish gelatine;
PVP (polyvinylpyrrolidone);
DMSO; and
formamide.

According to one embodiment, the amplification reaction buffer (d), which preferably is a PCR reaction buffer, comprises a soluble magnesium salt as Mg²⁺ source. The soluble the magnesium salt may be magnesium chloride or a chloride free magnesium salt such as magnesium sulfate or magnesium acetate.

### THE USE ACCORDING TO THE FIFTH ASPECT

In a fifth aspect, the present invention relates to the use of a kit according to the fourth aspect in the method according to the first, second and/or third aspect. Details of the respective kit and the methods are described in detail above and it is referred to the respective disclosure which also applies here.

### FURTHER EMBODIMENTS AND ITEMS ACCORDING TO THE PRESENT INVENTION

Also disclosed as part of the present invention are the following items of the technology of the present invention:

1. A method for preparing a biological sample for amplification based detection of at least one target nucleic acid comprised in the biological sample without prior target nucleic acid purification, comprising
contacting the biological sample with an extraction composition comprising
   (a) at least one surfactant,
   (b) at least one nuclease inhibitor, and
   (c) optionally at least one reducing agent, and
incubating the admixture comprising the biological sample in contact with the extraction composition to provide the prepared biological sample for amplification based detection of the target nucleic acid.

2. The method according to item 1, wherein the surfactant is selected from non-ionic and amphoteric surfactants.

3. The method according to item 1 or 2, wherein the surfactant is a non-ionic surfactant.

4. The method according to item 3, wherein the non-ionic surfactant is a polyoxyethylene-based non-ionic surfactant, preferably selected from the group consisting of
(aa) polyoxyethylene fatty acid esters, in particular polyoxyethylene sorbitan fatty acid esters;
(bb) polyoxyethylene fatty alcohol ether;
(cc) polyoxyethylene alkylphenyl ether, optionally wherein the polyoxyethylene alkyl phenyl ether is selected from the group consisting of polyoxyethylene nonylphenyl ether and polyoxyethylene isooctylphenyl ether; and
(dd) polyoxyethylene-polyoxypropylene block copolymers.

5. The method according to item 4, wherein the extraction composition comprises a polyoxyethylene fatty acid ester, comprising
a fatty acid derived from laureate, palmitate, stearate and oleate,
a polyoxyethylene component containing from 2 to 150, 4 to 100, 6 to 50 or 6 to 30 (CH₂CH₂O) units,
wherein preferably, the polyoxyethylene fatty acid ester is selected from polysorbate 20, polysorbate 40, polysorbate 60 and polysorbate 80.

6. The method according to item 4 or 5, wherein the extraction composition comprises a polyoxyethylene fatty alcohol ether, comprising
a fatty alcohol component having from 6 to 22 carbon atoms, and
a polyoxyethylene component containing from 2 to 150, 4 to 100, 6 to 50 or 6 to 30 (CH₂CH₂O) units,
wherein the polyoxyethylene fatty alcohol ether is preferably selected from the group consisting of polyoxyethylene lauryl ether, polyoxyethylene cetyl ether, polyoxyethylene stearyl ether and polyoxyethylene oleyl ether.

7. The method according to item 1 or 2, wherein the surfactant is an amphoteric surfactant, optionally a betaine such as N,N,N trimethylglycine.

8. The method according to any one of items 2 to 7, having one or more of the following characteristics:
(aa) wherein the extraction solution comprises the surfactant in a concentration that lies in a range of 0.1% to 30% (w/v), optionally selected from the ranges of 0.5% to 25% (w/v), 0.7% to 20% (w/v), 1% to 15% (w/v), 1.2% to 10% (w/v), 1.5% to 8% (w/v) and 2% to 5% (w/v); and/or
(bb) wherein the admixture comprising the biological sample in contact with the extraction composition comprises the surfactant in a concentration that lies in a range of 0.075% to 20% (w/v), optionally selected from the ranges of 0.1% to 15% (w/v), 0.15% to 15% (w/v), 0.2% to 10% (w/v), 0.25% to 8% (w/v), 0.3% to 5% (w/v), 0.35% to 3% (w/v) and 0.4% to 2% (w/v).

9. The method according to one or more of items 1 to 8, wherein the nuclease inhibitor is an RNase inhibitor or a DNase inhibitor, optionally wherein the extraction composition comprises two or more nuclease inhibitors, such as (i) two or more RNase inhibitors, (ii) two or more DNase inhibitors or (iii) one or more RNase inhibitors and one or more DNase inhibitors.

10. The method according to item 9, wherein a reverse transcription reaction and/or an amplification reaction can be performed in the presence of the comprised nuclease inhibitor.

11. The method according to item 9 or 10, wherein the nuclease inhibitor is an RNAase inhibitor.

12. The method according to item 11, wherein the RNase inhibitor is a proteinaceous RNase inhibitor.

13. The method according to item 12, wherein the RNase inhibitor is RNasin.

14. The method according to one or more of items 1 to 13, wherein the extraction composition comprises the reducing agent and wherein the reducing agent is capable of destroying disulfide bonds and denaturing proteins.

15. The method according to one or more of items 1 to 14, wherein the reducing agent assists in liquefying the biological sample.

16. The method according to one or more of items 1 to 15, wherein the reducing agent is selected from Tris(carboxyethyl)phosphine (TCEP), Dithiothreitol (DTT), N-acetyl cysteine, THPP (Tris(hydroxypropyl)phosphine), 1-thioglycerol and beta-mercaptoethanol.

17. The method according to item 16, wherein the extraction composition comprises Tris(carboxyethyl)phosphine (TCEP).

18. The method according to one or more of items 1 to 17, having one or more of the following characteristics:
(aa) wherein the extraction composition comprises the reducing agent in a concentration that lies in a range of 0.3 mM to 50 mM, optionally selected from the ranges of 0.5 mM to 25 mM, 1 mM to 20 mM, 1.5 mM to 15 mM and 2 mM to 10 mM or 2 mM to 5 mM; and/or
(bb) wherein the admixture comprising the biological sample in contact with the extraction composition comprises the reducing agent in a concentration that lies in a range of 0.1 mM to 15 mM, optionally selected from the ranges of 0.2 mM to 10 mM, 0.25 mM to 8 mM, 0.3 mM to 5 mM, 0.35 mM to 2 mM and 0.4 mM to 1.5 mM.

19. The method according to one or more of items 1 to 18, wherein the extraction composition comprise a reducing agent selected from Tris(carboxyethyl)phosphine (TCEP), Dithiothreitol (DTT), N-acetyl cysteine, THPP (Tris(hydroxypropyl)phosphine) and 1-thioglycerol in a concentration that lies in the range of 1 mM to 10 mM or 2 mM to 5 mM.

20. The method according to one or more of items 1 to 19, wherein the extraction composition is a liquid composition.

21. The method according to item 20, wherein the extraction solution is selected from the following embodiments:
(i) the extraction solution comprises
   (a) at least one non-ionic surfactant,
   (b) at least one proteinaceous RNase inhibitor, and
   (c) at least one reducing agent;
(ii) the extraction solution comprises
   (a) at least one polyoxyethylene-based non-ionic surfactant,
   (b) at least one proteinaceous RNase inhibitor, and
   (c) at least one reducing agent selected from Tris(carboxyethyl)phosphine (TCEP), Dithiothreitol (DTT), N-acetyl cysteine, THPP (Tris(hydroxypropyl)phosphine) and 1-thioglycerol;
(iii) the active ingredients of the extraction solution essentially consists of
   (a) a non-ionic surfactant, preferably a polyoxyethylene-based non-ionic surfactant,
   (b) a proteinaceous RNase inhibitor, and
   (c) a reducing agent, preferably selected from Tris(carboxyethyl)phosphine (TCEP), Dithiothreitol (DTT), N-acetyl cysteine, THPP (Tris(hydroxypropyl)phosphine) and 1-thioglycerol;
(iv) the extraction solution comprises
   (a) at least one polysorbate,
   (b) at least one proteinaceous RNase inhibitor, and
   (c) Tris(carboxyethyl)phosphine (TCEP);
(v) the active ingredients of the extraction solution essentially consists of
   (a) at least one polysorbate,
   (b) at least one proteinaceous RNase inhibitor, and
   (c) Tris(carboxyethyl)phosphine (TCEP).

22. The method according to item 20 or 21, wherein the extraction solution has a pH in the range of 6.0 to 9.0, optionally 6.0 to 8.5, 6.3 to 8.0 or 6.5 to 7.5.

23. The method according to any one of items 20 to 22, wherein the extraction solution is unbuffered.

24. The method according to any one of items 1 to 23, wherein the extraction composition does not comprise one or more, two or more, three or more or all of the following components:
an ionic surfactant;
a chaotropic salt;
chloride ions in a concentration exceeding 10 mM, wherein preferably the extraction solution does not comprise chloride ions;
an aliphatic C1-C5 alcohol; and/or
a proteinase enzyme.

25. The method according to any one of items 1 to 24, having one or more of the following characteristics:
(aa) the admixture is incubated for 1 to 60 min, 1 to 30 min, 1 to 20 min, 1 to 15 min, 1 to 10 min, 1.5 to 5 min or 2 to 3 min; and/or
(bb) preparing the admixture comprises agitating the biological sample in contact with the extraction composition, optionally wherein the admixture is aspirated and dispensed and/or vortexed for agitation;
(cc) the steps of contacting the biological sample with the extraction composition and incubating the admixture are carried out at ambient temperature and/or ice, optionally wherein all preparation steps prior to using the prepared biological sample for amplification based detection of the target nucleic acid are carried out at ambient temperature and/or ice.

26. The method according to any one of items 1 to 25, wherein the method for preparing the biological sample for amplification based detection of the target nucleic acid is characterized by one or more of the following features:
(aa) it does not involve heating the biological sample in contact with the extraction composition to a temperature ≥ 75° C., ≥ 70° C., ≥ 65° C., ≥ 60° C., ≥ 55° C., ≥ 50° C., ≥ 45° C. or ≥ 40° C. for at least 2 min prior to subjecting at least an aliquot or all of the prepared biological sample to an enzymatic reaction selected from reverse transcription and amplification;
(bb) it does not involve heating the biological sample in contact with the extraction composition to a temperature that would denature a comprised proteinaceous RNase inhibitor prior to subjecting at least an aliquot or all of the prepared biological sample to an enzymatic reaction selected from reverse transcription and amplification;
(cc) it does not involve centrifuging the prepared biological sample prior to subjecting at least an aliquot or all of the prepared biological sample to an enzymatic reaction selected from reverse transcription and amplification;
(dd) it does not involve removing cellular components from the prepared biological sample prior to performing an enzymatic reaction selected from reverse transcription and amplification; and/or
(ee) it does not comprise purifying the target nucleic acid prior to performing an enzymatic reaction selected from reverse transcription and amplification.

27. The method according to any one of items 1 to 26, wherein the biological sample that is contacted with the extraction composition is a pathogen heat-inactivated biological sample optionally comprised in a medium.

28. The method according to item 27, wherein the method comprises heating the biological sample in the absence of the extraction composition at a temperature suitable to inactivate pathogens prior to contacting the pathogen heat-inactivated biological sample with the extraction composition.

29. The method according to item 27 or 28, characterized by one or more of the following features:
(aa) heating for inactivating pathogens potentially comprised in the biological sample prior to contacting the heat-inactivated biological sample with the extraction composition comprises heating the biological sample at a temperature that inactivates pathogens;
(bb) heating for inactivating pathogens potentially comprised in the biological sample prior to contacting the heat-inactivated biological sample with the extraction composition comprises heating the biological sample to ≥50° C., ≥55° C. or ≥60, preferably ≥75° C., ≥80° C. or ≥ 85° C., more preferably ≥ 90° C. or ≥ 95° C.; and/or
(cc) heating for inactivating pathogens potentially comprised in the biological sample prior to contacting the heat-inactivated biological sample with the extraction composition comprises heating the biological sample in the collection container used for collecting the biological sample from the donor, optionally wherein
   (i) the biological sample is comprised in a medium in the collection container;
   (ii) the collection container has not been opened after collection of the biological sample and prior to heating for inactivating pathogens potentially comprised in the biological sample.

30. The method according to any one of items 27 to 29, wherein after heating, the pathogen heat-inactivated biological sample is contacted within ≤ 2 h, ≤ 1 h, ≤ 0.5 h or ≤ 20 min with the extraction composition.

31. The method according to any one of items 27 to 29, wherein the time span between heating the biological sample for providing the pathogen heat-inactivated biological sample and contacting the obtained pathogen heat-inactivated biological sample with the extraction composition is > 2 h, optionally wherein the time-span is within a range of > 2 h and ≤ 150 h, ≥ 3 h and ≤ 100 h or ≥ 4 h and ≤ 75 h.

32. The method according to item 30 or 31, wherein the pathogen heat-inactivated biological sample is put on hold, stored or transported prior to contacting the pathogen heat-inactivated biological sample with the extraction composition.

33. The method according to any one of items 1 to 32, having at least one of the following characteristics:
(aa) the at least one target nucleic acid is selected from RNA and/or DNA;
(bb) the at least one target nucleic acid is a pathogen-derived nucleic acid wherein the pathogen is selected from the group consisting of a virus, a bacterium, a protozoan, a viroid and a fungus;
(cc) the at least one target nucleic acid is a viral nucleic acid derived from a virus, preferably a RNA virus;
(dd) the at least one target nucleic acid is a viral RNA derived from an RNA virus;
(ee) the at least one target nucleic acid is derived from a coronavirus, in particular a coronavirus infectious for humans;
(ff) the target nucleic acid is provided by two or more target nucleic acids derived from the same pathogen.

34. The method according to item 33, wherein the at least one target nucleic acid is derived from a severe acute respiratory syndrome-related coronavirus, preferably severe acute respiratory syndrome coronavirus 2 (SARS-CoV-2) or severe acute respiratory syndrome coronavirus (SARS-CoV or SARS-CoV-1) or Middle East Respiratory Syndrome (MERS).

35. The method according to item 34, wherein the at least one target nucleic acid is a severe acute respiratory syndrome coronavirus 2 (SARS-CoV-2) derived nucleic acid.

36. The method according to item 35, wherein the one or more target nucleic acids are derived from SARS-CoV-2, optionally wherein the target nucleic acid sequences are derived from the SARS-CoV-2 genes N, N1, N2, RdRP, E and Orf1b.

37. The method according to any one of items 1 to 36, wherein the biological sample has one or more of the following characteristics:
(aa) it is a bodily sample;
(bb) it is a human sample;
(cc) it is a respiratory specimen, optionally collected from the upper or lower respiratory tract;
(dd) it is an oral sample, a nasal sample, a nasopharyngeal sample, an oropharyngeal sample, or a throat sample;
(ee) it is selected from the group consisting of saliva, sputum, spittle, mucus, drool, bronchoalveolar lavage, pharynx secretions, nasal secretions, nasopharyngeal secretions, salivary secretions, a swab or smear sample derived from mouth, nose and/or throat and a combination of the foregoing.

38. The method according to item 37, wherein the biological sample is selected from nasopharyngeal, oropharyngeal and nasal samples, preferably selected from a nasopharyngeal, oropharyngeal or nasal swab, smear or wash/aspirate samples, more preferably selected from swab or smear samples.

39. The method according to item 37, wherein the biological sample is selected from saliva, sputum and mucus.

40. The method according to any one of items 1 to 38, wherein the biological sample is contained in a medium.

41. The method according to item 40, having one or more of the following characteristics:
(aa) the biological sample is collected from the subject and directly placed into the medium or the biological sample is collected from the subject and after a delay, which optionally comprises storing and/or transporting the sample, is contacted with the medium to provide a biological sample contained in medium that is contacted with the extraction composition;
(bb) the medium is a transport medium, optionally a transport medium for swab and/or smear samples;
(cc) the medium is an aqueous solution;
(dd) the medium is a saline solution suitable to keep the osmotic pressure in cells comprised in the biological sample when the medium is in contact with the biological sample;
(ee) the medium stabilizes the at least one target nucleic acid against degradation;
(ff) the medium stabilizes cells and/or viral particles comprised in the biological sample; and/or
(gg) an aliquot of the medium containing the biological sample is contacted with the extraction composition.

42. The method according to item 40 or 41, wherein the medium has at least one of the following characteristics:
(aa) it comprises Hank’s balanced salt solution;
(bb) it is a salt containing solution;
(cc) it is a physiological salt solution;
(dd) it is a solution comprising 0.7% to 1.2% (w/v) or 0.8% to 1% (w/v) alkali metal salts;
(ee) it is a 0.9% (w/v) sodium chloride solution;
(ff) it is a phosphate buffer, optionally a PBS buffer.

43. The method according to any one of items 40 to 42, wherein the medium is a salt containing solution and wherein the total salt concentration in the medium lies in a range of 50 mM to 250 mM, such as 75 mM to 225 mM, 100 mM to 200 mM, 120 mM to 175 mM or 125 mM to 150 mM.

44. The method according to any one of items 40 to 43, wherein the medium comprises or consists of Hank’s balanced salt solution, Universal Transport Medium (UTM), Viral Transport Medium (VTM) or a medium having a total salt concentration in a range +/- 30% or +/- 20% compared to one or more of the aforementioned media.

45. The method according to one or more of items 40 to 44, wherein the method comprises
obtaining the biological sample contained in medium and optionally agitating the sample;
contacting at least an aliquot of the obtained biological sample contained in medium with the extraction composition thereby providing an admixture;
incubating the admixture to provide the prepared biological sample for amplification based detection of the target nucleic acid.

46. The method according to any one of items 1 to 45, wherein after incubation of the admixture comprising the biological sample, the extraction composition and optionally medium, the method further comprises
subjecting at least an aliquot or all of the prepared biological sample to an enzymatic reaction, preferably a reverse transcription and/or amplification reaction, and performing the enzymatic reaction.

47. The method according to item 46, wherein the enzymatic reaction comprises an amplification reaction, optionally wherein the amplification reaction has one or more of the following characteristics (i) it is a reverse transcription amplification reaction; (ii) it is a reverse transcription PCR; (iii) it is an isothermal amplification reaction; (iv) it is a polymerase chain reaction (PCR); (v) it is a quantitative PCR; (vi) it is a quantitative reverse transcription PCR; (vii) it is a digital PCR.

48. The method according to item 46 or 47, wherein the enzymatic reaction is a reverse-transcription amplification reaction, preferably a quantitative reverse-transcription polymerase chain reaction.

49. The method according to any one of items 46 to 48, wherein the steps of
contacting the biological sample with the extraction composition,
incubating the admixture comprising the biological sample in contact with the extraction composition, and
performing an enzymatic reaction, preferably a reverse-transcription amplification reaction or DNA amplification reaction,
are performed within the same reaction vessel.

50. The method according to any one of items 46 to 49, wherein the prepared biological sample that is subjected to the enzymatic reaction provides at least 20%, at least 30%, at least 40% or at least 45% of the total reaction volume of the enzymatic reaction, optionally wherein the prepared biological sample that is subjected to the enzymatic reaction provides up to 60% or up to 50% of the total reaction volume of the enzymatic reaction.

51. The method according to any one of items 1 to 50, wherein the method is for preparing a biological sample for amplification based detection of at least one pathogenic RNA target nucleic acid comprised in the biological sample without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture,
optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid;
subjecting at least an aliquot or all of the prepared biological sample to a reverse transcription and amplification reaction and performing the reaction.

52. The method according to any one of items 1 to 51, wherein the method is for preparing a biological sample for amplification based detection of at least one pathogenic RNA target nucleic acid without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture, wherein the admixture comprises
   (i) the non-ionic surfactant originating from the extraction solution in a concentration that lies in a range of 0.1% to 10% (w/v), optionally 0.2% to 5% (w/v) or 0.3% to 3% (w/v), and
   (ii) the reducing agent originating from the extraction solution in a concentration that lies in a range of 0.1 mM to 15 mM, optionally 0.2 mM to 10 mM, 0.3 mM to 5 mM or 0.4 mM to 1.5 mM;
optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid;
subjecting at least an aliquot or all of the prepared biological sample to a reverse transcription and amplification reaction and performing the reaction.

53. The method according to any one of items 1 to 52, wherein the method is for preparing a biological sample for amplification based detection of at least one pathogenic RNA target nucleic acid comprised in the biological sample without prior nucleic acid purification, comprising
contacting the biological sample with an extraction solution comprising
   (a) at least one non-ionic surfactant,
   (b) at least one proteinaceous RNase inhibitor, and
   (c) at least one reducing agent,
   to prepare an admixture, optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid;
subjecting at least an aliquot or all of the prepared biological sample to a reverse transcription and amplification reaction and performing the reaction,
wherein at least the steps of
contacting the biological sample with the extraction solution to prepare the admixture,
incubating the admixture, and
performing the reverse-transcription amplification reaction,
are performed within the same reaction vessel.

54. The method according to any one of items 1 to 53, wherein the method is for preparing a biological sample for amplification based detection of at least one pathogenic RNA target nucleic acid without prior nucleic acid purification, comprising
contacting the biological sample contained in medium with an extraction solution comprising
   (a) at least one non-ionic surfactant,
   (b) at least one proteinaceous RNase inhibitor, and
   (c) at least one reducing agent,
   to prepare an admixture, wherein the admixture comprises
   (i) the non-ionic surfactant originating from the extraction solution in a concentration that lies in a range of 0.1% to 10% (w/v), optionally 0.2% to 5% (w/v) or 0.3% to 3% (w/v), and
   (ii) the reducing agent originating from the extraction solution in a concentration that lies in a range of 0.1 mM to 15 mM, optionally 0.2 mM to 10 mM, 0.3 mM to 5 mM or 0.4 mM to 1.5 mM;
   optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture for at least 1 min, preferably at least 1.5 min or at least 2 min, to provide the prepared biological sample for amplification based detection of the at least one pathogenic RNA target nucleic acid;
subjecting at least an aliquot or all of the prepared biological sample to a reverse transcription and amplification reaction and performing the reaction;

wherein the prepared biological sample that is subjected to the reverse transcription and amplification reaction provides at least 30%, at least 40% or at least 45% of the total reaction volume of the reverse transcription and amplification reaction, and
wherein at least the steps of
   contacting the biological sample with the extraction solution to prepare the admixture,
   incubating the admixture, and
   performing the reverse-transcription amplification reaction,
   are performed within the same reaction vessel.

55. The method according to any one of items 1 to 54, wherein the method is for preparing a respiratory biological sample for amplification based detection of at least one pathogenic RNA target nucleic acid comprised in the biological sample without prior nucleic acid purification, comprising
contacting the respiratory biological sample contained in medium with an extraction solution comprising
   (a) at least one non-ionic surfactant,
   (b) at least one proteinaceous RNase inhibitor, and
   (c) at least one reducing agent,
   to prepare an admixture,
optionally wherein the method comprises heating the respiratory biological sample contained in medium in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid;
subjecting at least an aliquot or all of the prepared biological sample to a reverse transcription and amplification reaction and performing the reaction for detecting the presence or absence of the at least one pathogenic RNA target nucleic acid;

wherein the prepared biological sample that is subjected to the reverse transcription and amplification reaction provides at least 30% or at least 40% of the total reaction volume of the reverse transcription and amplification reaction, which preferably is a quantitative RT-PCR reaction, and
wherein at least the steps of
   contacting the respiratory biological sample contained in medium with the extraction solution to prepare the admixture,
   incubating the admixture, and
   performing the reverse-transcription amplification reaction,
   are performed within the same reaction vessel, and
wherein the reverse transcription and amplification reaction comprises primers suitable for amplifying one or more, preferably two or more, target nucleic acids derived from a severe acute respiratory syndrome-related coronavirus, preferably severe acute respiratory syndrome coronavirus 2 (SARS-CoV-2).

56. A method for amplification based detection of at least one target nucleic acid comprised in a biological sample without prior purification of the target nucleic acid, comprising
(A) preparing the biological sample for amplification based detection of the target nucleic acid, wherein preparing comprises
   contacting the biological sample with an extraction composition comprising
      (a) at least one surfactant,
      (b) at least one nuclease inhibitor, and
      (c) optionally at least one reducing agent, and
   incubating the admixture comprising the biological sample in contact with the extraction composition to provide the prepared biological sample;
(B) subjecting at least an aliquot or all of the prepared biological sample to an amplification reaction and amplifying the at least one target nucleic acid, optionally wherein a reverse transcription reaction is performed in order to reverse transcribe RNA to cDNA prior to amplification.

57. The method according to item 56, wherein the at least one target nucleic acid is derived from a pathogen.

58. A method for detecting the presence or absence of a pathogen in a biological sample based on amplifying at least one target nucleic acid derived from the pathogen, comprising
(A) preparing the biological sample for amplification based detection of the target nucleic acid, wherein preparing comprises
   contacting the biological sample with an extraction composition comprising
      (a) at least one surfactant,
      (b) at least one nuclease inhibitor, and
      (c) optionally at least one reducing agent, and
   incubating the admixture comprising the biological sample in contact with the extraction composition to provide the prepared biological sample;
(B) subjecting at least an aliquot or all of the prepared biological sample to an amplification reaction and amplifying the at least one target nucleic acid, optionally wherein a reverse transcription reaction is performed in order to reverse transcribe RNA to cDNA prior to amplification.

59. The method according to any one of items 56 to 58, wherein (A) preparing the biological sample for amplification based detection of the target nucleic acid is performed as defined in any one of items 1 to 55 and wherein preferably, in (A) the biological sample is contacted with an extraction composition as defined in any one of items 2 to 24.

60. The method according to any one of items 56 to 59, wherein the at least one target nucleic acid is as defined in any one of items 33 to 36, wherein preferably the one or more target nucleic acids are RNA targets and wherein the amplification reaction is a reverse transcription amplification reaction.

61. The method according to any one of items 56 to 59, wherein one or more SARS-CoV-2 target nucleic acids are reverse transcribed and amplified for detecting the presence of absence of SARS-CoV-2 in the biological sample, wherein the target nucleic acid sequences are derived from the SARS-CoV-2 genes N, N1, N2, RdRP, E and Orf1b.

62. The method according to any one of items 56 to 61, characterized by one or more, or two or more of the following features
(aa) the biological sample is a biological sample as defined in any one of items 37 to 39;
(bb) the biological sample is comprised in a medium as defined in any one of items 40 to 44, optionally wherein preparing in (A) comprises
   obtaining the biological sample contained in medium and optionally agitating the sample;
   contacting at least an aliquot of the obtained biological sample contained in medium with the extraction composition thereby providing an admixture;
   incubating the admixture to provide the prepared biological sample for amplification based detection of the target nucleic acid;
(cc) the method comprises heating the biological sample in the absence of the extraction composition at a temperature suitable to inactivate pathogens prior to contacting the pathogen heat-inactivated biological sample with the extraction composition, wherein if such heating step is carried out, the heating of the biological sample is preferably performed as defined in item 29, optionally wherein the pathogen heat-inactivated biological sample is further processed as defined in any one of items 30 to 32.

63. The method according to any one of items 56 to 62, wherein
(aa) the prepared biological sample that is subjected to the amplification reaction provides at least 20%, at least 30%, at least 40% or at least 45% of the total reaction volume of the amplification reaction, optionally wherein the prepared biological sample that is subjected to the amplification reaction provides up to 60% or up to 50% of the total reaction volume of the amplification reaction; and/or
(bb) wherein at least the steps of
   contacting the biological sample with the extraction solution to prepare the admixture,
   incubating the admixture, and
   performing the reverse-transcription amplification reaction,
   are performed within the same reaction vessel.

64. The method according to any one of items 56 to 63, wherein the biological sample is a respiratory biological sample contained in medium and the method is for amplification based detection of at least one pathogenic RNA target nucleic acid comprised in the biological sample without prior nucleic acid purification,
wherein the extraction composition used in (A) is an extraction solution comprising (a) a non-ionic surfactant, (b) a proteinaceous RNase inhibitor, and (c) a reducing agent, optionally wherein the extraction solution consists essentially of or consists of the aforementioned active ingredients comprised in liquid;
wherein in (B) a reverse transcription and amplification reaction, preferably a quantitative reverse transcription PCR, is performed for detecting the presence or absence of the at least one pathogenic RNA target nucleic acid;

wherein the prepared biological sample that is subjected to the reverse transcription and amplification reaction provides at least 25%, at least 30% or at least 40% of the total reaction volume of the reverse transcription and amplification reaction, and
wherein at least the steps of
   contacting the respiratory biological sample contained in medium with the extraction solution to prepare the admixture,
   incubating the admixture comprising the extraction solution, the biological sample, and medium to provide the prepared biological sample, and
   performing the reverse-transcription amplification reaction,
   are performed within the same reaction vessel, and
wherein the reverse transcription and amplification reaction comprises primers suitable for amplifying one or more, preferably two or more, target nucleic acids derived from a severe acute respiratory syndrome-related coronavirus, preferably severe acute respiratory syndrome coronavirus 2 (SARS-CoV-2).

65. A kit for performing a method as defined in any one of items 1 to 64 or items 71 to 79, comprising

(a) an extraction composition as defined in any one of items 1 to 24.

66. The kit according to item 65, comprising one or more and preferably all of the following components:
(b) a DNA polymerase;
(c) a reverse transcriptase;
(d) an amplification reaction buffer comprising a Mg²⁺ source, a buffering agent and optionally further additives;
(e) nucleotides, preferably a dNTP mix; and
(f) primers for amplifying the at least one target nucleic acid,
optionally wherein components (b) to (e) or (b) to (f) are comprised in a single composition.

67. The kit according to item 66, wherein the kit comprises one or more of the following additives which are preferably comprised in the amplification reaction buffer (d):
an ammonium salt, optionally selected from ammonium sulfate and ammonium chloride;
polyethylene glycol;
N,N,N-trimethylglycine;
serum albumin;
a metal ion chelator, optionally EGTA;
glycerol;
fish gelatine;
PVP (polyvinylpyrrolidone);
DMSO; and
formamide.

68. The kit according to item 66 or 67, wherein the amplification reaction buffer (d), which preferably is a PCR reaction buffer, comprises a magnesium salt as Mg²⁺ source, optionally wherein the magnesium salt is selected from magnesium chloride or a chloride free magnesium salt such as magnesium sulfate and wherein preferably, the amplification reaction buffer (d) has any one of the characteristics as defined in any one of items 73 to 77.

69. The kit according to any one of items 65 to 68, wherein all components (b) to (e) are comprised in a single composition providing a amplification master mix, wherein preferably, the amplification master mix has any one of the characteristics as defined in any one of items 73 and 78 or 79.

70. The kit according to any one of items 65 to 69, comprising a digestion solution comprising a proteolytic enzyme and a reducing agent, optionally wherein the proteolytic enzyme is a protease, preferably proteinase K and the reducing agent is selected from Tris(carboxyethyl)phosphine (TCEP), Dithiothreitol (DTT) and beta-mercaptoethanol, preferably Tris(carboxyethyl)phosphine (TCEP).

71. The method according to any one of items 56 to 64, wherein subjecting at least an aliquot or all of the prepared biological sample to an amplification reaction in (B) comprises contacting the prepared biological sample with the components used for performing the amplification or reverse transcription amplification reaction thereby providing an amplification reaction admixture and wherein the prepared amplification reaction admixture comprises:
(a) the prepared biological sample;
(b) a DNA polymerase;
(c) optionally a reverse transcriptase, which is included in case a reverse transcription amplification is performed;
(d) an amplification reaction buffer comprising a Mg²⁺ source, a buffering agent and optionally further additives;
(e) nucleotides, preferably a dNTP mix, optionally wherein the nucleotides comprise modified nucleotides or dUTP; and
(f) primers for amplifying the one or more target nucleic acids and optionally probes.

72. The method according to item 71, wherein the method comprises contacting the prepared biological sample with an amplification master mix comprising components (b) to (e) and separately provided primers for amplifying the one or more target nucleic acids; and optionally probes which is advantageous in case a quantitative amplification is performed.

73. The method according to item 71 or 72, wherein the ionic strength of the amplification reaction buffer (d) or the amplification master mix comprising components (b) to (e) is reduced to thereby compensate the introduction of ions, in particular ions derived from alkali metal salts and/or chlorides, into the amplification reaction admixture due to the prepared biological sample that may contain the medium.

74. The method according to any one of items 71 to 73, wherein the amplification reaction buffer (d) has one or more, preferably two or more, more preferably three of more of the following characteristics:
(aa) the amplification reaction buffer (d) does not comprise sodium chloride in a concentration ≥ 30 mM, optionally wherein it does not comprise sodium chloride in a concentration ≥ 20 mM, ≥ 15 mM, ≥ 10 mM or ≥ 5 mM and wherein preferably, the amplification reaction buffer (d) contains no sodium chloride;
(bb) the amplification reaction buffer (d) does not comprise potassium chloride in a concentration ≥ 30 mM, such as ≥ 20 mM, ≥ 15 mM, ≥ 10 mM or ≥ 5 mM and wherein preferably, the amplification reaction buffer (d) contains no potassium chloride;
(cc) the amplification reaction buffer (d) does not comprise potassium chloride or sodium chloride;
(dd) the alkali metal chloride concentration in the amplification reaction buffer (d) is ≤ 30 mM, ≤ 20 mM, ≤ 15 mM or ≤ 10 mM, wherein preferably, the amplification reaction buffer (d) does not contain alkali metal chlorides;
(ee) the alkali metal salt concentration in the amplification reaction buffer (d) is ≤ 30 mM, such as ≤ 20 mM, ≤ 15 mM or ≤ 10 mM and wherein preferably, the amplification reaction buffer (d) does not contain alkali metal salts.

75. The method according to any one of items 71 to 74, wherein the amplification reaction buffer (d) comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)propanesulphonic acid, N-(2-hydroxyethyl)piperazine-N′-(2-ethanesulphonic acid), piperazine-1,4-bis(2-ethanesulphonic acid), N-cyclohexyl-2-aminoethanesulphonic acid and 2-(N-morpholino)ethanesulphonic acid. Preferably, the buffering agent is selected from tris(hydroxymethyl)aminomethane and 3-(N-morpholino)-propanesulphonic acid.

76. The method according to any one of items 71 to 75, wherein the pH of the amplification reaction buffer (d) is adjusted with an acid that does not comprise chloride, optionally wherein the pH is adjusted with an organic acid, preferably a carboxylic acid.

77. The method according to any one of items 71 to 76, wherein the amplification reaction buffer (d) is characterized in that:
(i) it does not comprise potassium chloride or sodium chloride;
(ii) it comprises a buffering agent that does not comprise chloride ions, optionally wherein the buffering agent is selected from tris(hydroxymethyl)aminomethane and 3-(N-morpholino)-propanesulphonic acid, and
(iii) the pH of the amplification reaction buffer (d) is adjusted with an organic acid, preferably a carboxylic acid.

78. The method according to any one of items 72 to 78, wherein the amplification master mix comprising components (b) to (e) has one or more, preferably two or more or more preferably three or more of the following characteristics:
(aa) it does not comprise sodium chloride in a concentration ≥ 50 mM, ≥ 20 mM, ≥ 15 mM or ≥ 10 mM and wherein preferably, it contains no sodium chloride;
(bb) it does not comprise potassium chloride in a concentration ≥ 100 mM, ≥ 75 mM, ≥ 60 mM or ≥ 50 mM, wherein optionally it contains no potassium chloride;
(cc) the alkali metal chloride concentration in the amplification master mix is ≤ 100 mM, ≤ 75 mM, ≤ 60 mM, ≤ 50 mM or ≤ 45 mM;
(dd) The alkali metal salt concentration in the amplification master mix is ≤ 100 mM, ≤ 75 mM, such as ≤ 60 mM, ≤ 50 mM or ≤ 45 mM;
(ff) the chloride ion concentration is ≤ 250 mM, preferably ≤ 200 mM, ≤ 175 mM or ≤ 150 mM; optionally wherein said amplification master mix is provided in concentrated form as 3x or 4x amplification master mix.

79. The method according to any one of items 72 to 79, wherein the amplification master mix comprising components (b) to (e) may have one or both of the following characteristics:
(aa) it comprises a buffering agent that does not comprise chloride ions, optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)propanesulphonic acid, N-(2-hydroxyethyl)piperazine-N′-(2-ethanesulphonic acid), piperazine-1,4-bis(2-ethanesulphonic acid), N-cyclohexyl-2-aminoethanesulphonic acid and 2-(N-morpholino)ethanesulphonic acid and preferably is selected from tris(hydroxymethyl)aminomethane and 3-(N-morpholino)propanesulphonic acid;
(bb) the pH is adjusted with an organic acid, preferably a carboxylic acid, optionally wherein the carboxylic acid selected from acetic acid, formic acid, propionic acid and butyric acid, preferably acetic acid.

80. Use of a kit according to any one of items 65 to 69 or 70 in a method as defined in any one of items 1 to 64 or items 71 to 79.

As is apparent from the above disclosure and the provided examples, the present invention provides a streamlined workflow for the preparation of crude biological samples for amplification based detection of target nucleic acids and pathogens without prior nucleic acid purification which inter alia enables an immediate and fast real-time PCR run. The present invention enables a straightforward workflow. Several advantageous embodiments have been described above and are illustrated in the examples. An aliquot may be taken from a primary sample, such as a nasopharyngeal, an oropharyngeal or a nasal swab, comprised in transport media, such as Universal Transport Media (UTM™), and contacted with the extraction solution of the present invention that is particularly suitable to prepare viral nucleic acids, including viral RNA, without degradation. The admixture comprising the biological sample in transport media and the extraction solution of the invention is then combined with the components of the amplification reaction, which preferably is a reverse transcription amplification. Other options are also disclosed herein. As disclosed herein, a routine real-time PCR can be performed using the prepared biological sample without prior purification which provides reliable and sensitive results. Advantageously any cycler can be used and the overall rapid workflow of the invention allows to deliver results in under one hour. Thus, the present invention significantly simplifies and accelerates PCR analysis compared to standard extraction-based quantitative PCR processes, which e.g. require three hours and more to obtain a result. This enables laboratories to significantly increase the frequency of pathogen tests. The level of detection that can be achieved with the method of the present invention is similar to or better than regular PCR workflows and its performance compares to standard public health protocols of the U.S. Centers of Disease Control (CDC), the World Health Organization (WHO) and others that use the gold standard for sample extraction. Furthermore, the present invention is compatible with standard laboratory automation equipment, standard assay and transport media and allows to combine the reagents for sample preparation and target detection in one kit. Furthermore, significant cost savings are possible by reducing plastic and reagent use as well as laboratory utilization. Overall, the present invention which is based on the use of the extraction composition according to the present invention in order to prepare the crude biological sample for amplification based detection of the target nucleic acids removes key testing bottlenecks for pathogen detection, such as SARS-CoV-2 and other RNA viruses, by significantly simplifying and accelerating standard extraction-based PCR processes.

Furthermore, the methods disclosed hereon allow the parallel detection of different pathogens in a multiplex format and also allow the genotyping of different variants of a pathogen, such as different virus variants. These are also advantageous applications of the methods according to the present invention.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

As used in the subject specification and claims, the singular forms “a”, “an” and “the” include plural aspects unless the context clearly dictates otherwise. The terms “include,” “have,” “comprise” and their variants are used synonymously and are to be construed as non-limiting. Further components and steps may be present. Throughout the specification, where compositions are described as comprising components or materials, it is additionally contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

The present application claims priority of the following applications EP 20 200 426.3 of Oct. 6, 2021, EP 20 200 425.5 of Oct. 6, 2020, US 63/088,423 of Oct. 6, 2021 and EP 20 214 412.7 of Dec. 16, 2020 the content of which is herein incorporated by reference in its entirety.

### EXAMPLES

It should be understood that the following examples are for illustrative purpose only and are not to be construed as limiting this invention in any manner.

The following examples demonstrate the superior performance of the direct PCR system according to the present invention.

Abbreviations:

| | |
|---|---|
| QN | QuantiNova |
| Patho | QuantiNova Pathogen Reaction Mix |
| PCR | polymerase chain reaction |
| RT | reverse transcriptase enzyme |
| NADB | QuantiTect Nucleic Acid Dilution Buffer (from QuantiTect Virus Kit) |
| NaCl | Sodium chloride |
| KCl | Potassium chloride |
| HCl | Hydrochloric acid |
| NaAc | Sodium acetate |
| KAc | Potassium acetate |
| UTM | Universal Transport Medium |
| VTM | Viral Transport Medium (Centers for Disease Control and Prevention; preparation and composition of VTM published as SOP#: DSR-052-05, including attachments #1 and #2, herein incorporated by reference) |
| PBS | Phosphate Buffered Saline |
| HBSS | Hank’s Balanced Salt Solution |
| FCS | fetal calve serum |
| IC | internal control |
| SDS | Sodium dodecyl sulfate |
| STAT | QIAstat-Dx instrument |
| DTT | Dithiothreitol |
| TCEP | Tris(2-carboxyethyl)phosphine |
| ACC | N-Acetyl-Cysteine |
| QN IC RNA | Internal control RNA from the QuantiNova Pathogen Kit (QIAGEN) |
| FAM | 6-Carboxyfluorescein |
| Ct | cycle threshold |
| BSA | bovine serum albumin |
| ES | extraction solution |
| RSV | human respiratory virus |
| Flu A | Influcenza A virus |
| Flu B | Influenza B virus |
| NTC | non-template control |
| LoD | level of detection |
| HR | hit rate |

If not otherwise mentioned, experiments were performed with the QuantiNova Pathogen + IC Kit (QIAGEN, Hilden), herein incorporated by reference. The QuantiNova Pathogen Master Mix used for preparing the PCR reaction admixture comprises the PCR components presented in Table 1 as is also indicated in the QuantiNova Pathogen + IC Kit Handbook (QIAGEN, May 2016):

TABLE 1

Core components of the QuantiNova Pathogen Master Mix as indicated in the QuantiNova Pathogen + IC Kit Handbook (QIAGEN, May 2016)

| | |
|---|---|
| Component | Description |
| QuantiNova DNA Polymerase | Modified Taq polymerase (recombinant 94 kDa DNA polymerase) |
| HotStartRT-Script Reverse Transcriptase | Modified form of a recombinant 77 kDa reverse transcriptase |
| QuantiNova Pathogen Buffer (also referred to herein as QN reaction buffer or QN PCR buffer) | Tris-HCl |
| | KCl |
| | NH₄Cl |
| | MgCl₂ |
| | additives enabling fast cycling |
| dNTP mix | dATP, dCTP, dGTP, dTTP |

In order to improve the PCR reaction conditions and enable the direct amplification of pathogen target nucleic acids from crude biological samples (to thereby avoid prior nucleic acid isolation), modified versions of the QuantiNova Pathogen Master Mix were prepared. These modified versions of the QuantiNova Pathogen Master Mix were prepared by modifying the salt containing QN reaction buffer as is detailed in the below examples, whereby the composition of the master mix is changed. Unless indicated otherwise in the below examples, the amplification protocol of the QuantiNova Pathogen + IC Kit was followed as it is disclosed in the 2016 handbook (QIAGEN, Hilden). All reaction volumes were 20 µl in total following the manufacturer’s instructions. A maximum of 12 µl sample input is possible. When less than 12 µl sample were added, volume was adjusted with water unless indicated otherwise. Amplification took place for 40 cycles.

In initial experiments PCR and RT-PCR were evaluated separately to detect effects on the reverse transcriptase or DNA polymerase, respectively.

Human genomic DNA was used as template material with the IC assay from the Investigator QuantiPlex Pro Kit (QIAGEN, Hilden) for the PCR. For RT-PCR the Internal Control RNA from the QuantiNova Pathogen Kit (QIAGEN) was used.

All targets and assays are listed in the respective experiment and suitable primers were included in the PCR reaction admixture to allow amplification of the targets.

As targets were used either appropriate in vitro transcripts or – especially for the described sample lysis/preparation tests – iMS2 phages or inactivated virus particles (NATtrol™ SARS-Related Coronavirus 2 (SARS-CoV-2) Stock, #NATSARS(COV2)-ST; SARS-Related Coronavirus 2 (SARS-CoV-2) Isolate: USA-WA1/2020 Culture Fluid (Heat Inactivated), # 0810587CFHI; Zeptosens, Buffalo, USA) as mentioned in the respective example.

SARS-CoV-2 Assays performed in the following experiments rely on target sequences for the SARS-CoV-2 genes N1 and N2 published by the US CDC (retrieved on Sep. 30, 2020 at https://www.cdc.gov/coronavirus/2019-ncov/lab/rt-per-panel-primer-probes.html, last updated May 29, 2020) as well as RdRP, E and Orf1b as published by the Charité (Corman, VM et al., Euro Surveill, Jan. 23, 2020).

### Example 1: Effect of Non-Ionic Surfactants on PCR and RT-PCR Amplification (I)

To increase the sensitivity of the RT-PCR reaction, the access to the target RNA has to be as complete as possible. It is known in the art that surfactants will improve the lysis of viral particles. However, due to their “solubilization abilities”, surfactants will also have a negative impact on all proteins including the enzymes used for reverse transcription and amplification, such as the reverse transcriptase and DNA polymerase and therefore presumably interfere with optimal amplification conditions for RNA and DNA target nucleic acids.

To evaluate possible effects on these enzymes, the non-ionic surfactant Tween20 (polysorbate 20) was tested in increasing amounts in the amplification reaction admixture.

### Results:

Surprisingly, no negative effect on both the reverse transcriptase and the Taq polymerase was observed with the non-ionic polysorbate. This makes this type of surfactant (non-ionic) an ideal candidate for a possible “extraction solution” which will improve the lysis of the biological sample and e.g. contained virus particles in order to render the target nucleic acids accessible without having an inhibitory effect on the amplification reaction.

### Example 2: Effect of Non-Ionic Surfactants on PCR and RT-PCR Amplification (II)

To demonstrate that non-ionic surfactants in general are ideal components for sample lysis without inhibiting the amplification reaction (e.g. reverse transcription PCR or PCR) different non-ionic surfactants were tested.

In the following experiment increasing amounts of different non-ionic surfactants belonging to the group of polyoxyethylene fatty acid esters and polyoxyethylene fatty alcohol ether were tested. For this purpose, Tween20 (polysorbate 20), Tween60 (polysorbate 60), and Brij58 (polyoxyethylene(20) cetyl ether) were added in different final concentrations to the PCR (left column, blue) and RT-PCR (right column, red) reaction to test for any inhibitory effects on the RT-PCR or PCR reaction.

For Tween60 and Brij58 higher concentrations were not tested to ensure good viscosity and solubility.

### Results:

Example 2 demonstrates the compatibility of different non-ionic surfactants with the amplification reaction making non-ionic surfactants ideal candidates for surfactants used in a lysis system that allows subsequent direct amplification without the need for prior target nucleic acid purification.

Even with very high concentrations of Tween20 up to 25% (w/v) in the amplification reaction admixture, only a minimal inhibition was observed.

### Example 3: Effect of RNase Inhibition (I)

Sample type: oropharyngeal swab in UTM
Target: in vitro transcript; 5000 copies
Assay: N2 (US-CDC)

Real life biological samples contain high amounts of nucleases due to lysed human cells and the FCS that is often comprised as part of the transport medium. As is demonstrated below, it is therefore advantageous to include a nuclease inhibitor into the extraction solution in order to increase the sensitivity for target nucleic acid detection. RNA is particularly prone to nuclease degradation and RNA targets are very important for pathogen detection, such as coronavirus detection.

To demonstrate the beneficial effect of stability of target RNA by an upstream “extraction solution” containing either an RNase inhibitor (from the QlAseq UPX3-Trancriptome Kit (QIAGEN): 0.5 U in final RT-PCR reaction) or water, an “extraction solution” comprising the RNase inhibitor was added to real life biological samples (negative for SARS-CoV-2) spiked with an in vitro transcript and the mixture was additionally heated for 5 min at 95° C. as described in several publications (e.g. Foomsgard and Rosenstierne (2020)) followed by incubation on ice.

### Results:

The direct comparison with and w/o a nuclease inhibitor, here a RNase inhibitor in view of the RNA target, clearly shows the benefit of an upstream “extraction solution” with an RNase-inhibiting substance when targeting an RNA for pathogen detection.

When detecting a DNA target nucleic acid, the “extraction solution” may comprise a DNase inhibitor suitable for DNase inhibition. Furthermore, the “extraction solution” may comprise an RNase inhibitor and a DNase inhibitor in order to improve the detection of both types of target nucleic acids.

### Example 4: Effect of RNase Inhibition (II)

Sample type: UTM
Target: in vitro transcript; 5, 50, 500 copies
Assay: N2 (US CDC)

The RT-PCR was performed with a modified version of the QN Pathogen Master Mix that was prepared in accordance with the previous examples. The PCR reaction buffer used to set up the amplification master mix contained no KCl and no NaCl (subsequently referred to as PCR reaction buffer w/o KCl/NaCl). It was free of alkali metal salts. The pH was adjusted with acetic acid. For the ease of simplicity, this set-up that was also used in the following examples (and Example 3) is also referred to subsequently as modified QN Pathogen Master Mix w/o KCl/NaCl and pH adjusted with acetic acid.

To demonstrate the general benefit of an upstream “extraction solution” containing a RNase inhibitor (from the QlAseq UPX3-Trancriptome Kit (QIAGEN): 0.5 U in final RT-PCR reaction), different lots of UTM were incubated for 5, 10 and 30 min on ice in the presence of an “extraction solution” containing the RNase inhibitor or in water. The effect was tested with 5, 50, 500 copies of an in vitro RNA target representing the N2-gene, respectively.

### Results:

The results of this Example confirm the initial findings of the previous Example. Adding an “extraction solution” with an RNase inhibitor to the sample improves the sensitivity dramatically (compare signals of the UTM lots with and w/o RNase inhibitor) when a crude sample is directly used for virus detection in an amplification reaction.

### Example 5: Combining Positive Effects to an “Extraction Solution”

Sample type: oropharyngeal swab in UTM
Target: in vitro transcript (500, 5000 copies)
Assays: N2 (US-CDC)

RT-PCR was performed with a modified QN Pathogen Mix w/o KCl/NaCl and pH adjusted with acetic acid (see above).

The previous examples showed positive effects with regard to sensitivity for detecting an RNA target nucleic acid when an RNase inhibitor is used for preparing the biological sample for the amplification reaction, here a reverse transcription PCR. As noted above, it is also desirable to include a lysis-promoting reagent like a surfactant to improve the access to the target nucleic acid improving the sensitivity even more. Non-ionic surfactants such as Tween20 have already been demonstrated for not having any negative effects on the PCR performance (see prior Examples). In the following, it was therefore analysed that there is also no disadvantageous effect on a proteinaceous RNase inhibitor which constitutes a preferred RNase inhibitor because of the strong RNase inhibition capacity.

Based on these previous findings an “extraction solution” was composed to improve sample lysis without a negative effect on the subsequent PCR reaction. “Extraction solution” (final concentration in PCR):
0.5 U RNase Inhibitor (QIAGEN, from QlAseq UPX3-Transcriptomics Kit): 40 U
0.15% Tween20: 0.3 µl of a 10% Tween20 stock solution were added to each PCR reaction with a total volume of 20 µl.

In addition, heating can have a positive effect on lysis. Therefore the samples were either incubated (1) on ice or (2) heated at 45° C. for 5 min (non-denaturing condition for RNase inhibitor) or (3) heated at 95° C. for 5 min (denaturing condition for RNase inhibitor).

### Results:

These results again clearly showed the dramatic effect of an RNase inhibitor in the “extraction solution” on the sensitivity of the RT-PCR.

Even more, the data also showed that heating under non-denaturing conditions has practically no effect on the Ct-values when an RNase inhibitor is present (compare results on ice with results at 45° C. heating). On the other hand, heating at 95° C., which resulted in denaturation and therefore inactivation of the proteinaceous RNase inhibitor, led to increased Ct-values, thus indicating a disadvantageous effect of high temperatures when using a RNase inhibitor.

### Example 6: Suitability of Different RNase Inhibitors

Sample type: oropharyngeal swab in UTM
Target: in vitro transcript
Assay: N2 (US CDC)

RT-PCR was performed with a modified QN Pathogen Mix w/o KCl/NaCl and pH adjusted with acetic acid (see above).

To demonstrate that the positive effect of a RNase inhibitor is especially favorable and that the effect is independent of a special RNase inhibitor, three different RNase inhibitors, each having a final concentration of 0.5 U in the PCR, were compared against a standard protocol without RNase inhibitor. To quantify the results, real life samples spiked with the target nucleic acid were used.

The sample was spiked with the target, an “extraction solution” as described in the previous examples was added and the mixture was heated at 45° C. and 85° C. for 5 min each and directly applied to the RT-PCR reaction. A non-heated sample which was directly applied to the PCR without a previous incubation step was used as control.

### Results:

This example demonstrated that the advantageous effect of the RNase inhibitor is independent of a specific RNase inhibitor and that different RNase inhibitors may be used. As disclosed herein, proteinaceous RNase inhibitors are particularly advantageous and different types of proteinaceous RNase inhibitors are commercially available and thus readily available.

As already outlined in the previous experiment, a heating step prior to the amplification reaction showed no positive effect with regard to sensitivity under conditions where no denaturation of the RNase inhibitors was achieved.

### Example 7: Advantages of Using Reducing Agents

Sample type: oropharyngeal swab in UTM
Target: in vitro transcript
Assay: N2 (US CDC)

In this example it was tested whether agents disturbing the tertiary structure of enzymes by breaking internal disulfide bonds could support the inhibition of RNases and improve RNA stability during sample preparation and therefore the detection sensitivity in a direct amplification protocol, where accordingly, the components of the extraction solution are transferred into the amplification reaction. In standard RNA sample preparation and purification methods, e.g. RNeasy (QIAGEN), where beta-mercaptoethanol is added to the lysis buffer, the reducing agent is removed during the subsequent purification protocol.

In a direct amplification, disulfide-breaking agents must be identified which selectively impair the destructive enzymes (e.g. nucleases such as in particular RNases) and at the same time do not influence the active structure of the reverse transcriptase and/or the DNA polymerase and thus the enzymes that are subsequently used for target detection by amplification.

Different typical disulfide reducing agents were thus tested for their influence on (RT-)PCR efficiency. RT-PCR was performed with a modified QN Pathogen Mix w/o KCl/NaCl and pH adjusted with acetic acid (see above).

### Results:

None of the tested reducing agents showed a negative effect on the performance of the PCR and RT-PCR reaction at the tested concentrations in the reaction mixture.

Because disulfide reducing agents like TCEP, N-acetyl-L-cysteine, DTT, or beta-mercaptoethanol have a positive effect on RNA stability due to their ability to disturb (disulfide bond-containing) nucleases and no negative effect was observed in the amplification reaction (PCR and reverse transcription PCR), TCEP or a similar disulfide reducing agent is preferably included into the “extraction solution” used for biological sample preparation.

### Example 8: Individual Effects of Different Additives in the “Extraction Solution”

In a subsequent experiment the effects of reducing agents and some other additives on the amplification reaction were tested. Positive patient samples were diluted in a negative patient sample.
Sample type: oropharyngeal swab in UTM
Target: in vitro transcript (500, 5000 copies)
Assay: N2 (US CDC)

RT-PCR was performed with a modified QN Pathogen Mix w/o KCl/NaCl and pH adjusted with acetic acid (see above).

For each amplification reaction, 9 µl of sample were mixed with different versions of 1 µl of 10x “extraction solution”, thereby providing 10 µl prepared biological sample for the amplification reaction.

0.5 U RNase inhibitor (from the QlAseq UPX3-Trancriptome Kit (QIAGEN)) plus the following additives in the concentrations present in the “extraction solution” as indicated in Table 2.

TABLE 2

Agents added to the RNase inhibitor in the concentrations present in the “extraction solution”
No additive
0.3 mM DTT
0.6 mM DTT
0.9 mM DTT
0.3 mM ACC
0.6 mM ACC
0.9 mM ACC
0.3 mM TCEP
0.6 mM TCEP
0.9 mM TCEP
0.1% Tween20
0.5% Tween20
1% Tween20
betaine 100 mM
betaine 200 mM
betaine 350 mM
2 mg/ml BSA/ 0.2%
3 mg/ml BSA/ 0.3%
4 mg/ml BSA/ 0.4%

### Results:

None of the tested additives showed a significant change in the sensitivity of the detection reaction. Thus, they did not negatively affect the RNase inhibitor or the amplification reaction and are thus suitable as components for the “extraction solution” to support the preparation of the biological sample for direct amplification without prior purification.

### Example 9: Synergistic Effects of TCEP and Tween20

Sample type: oropharyngeal swab in UTM (positive sample 1:160 diluted in two different negative sample)
Target: SARS-CoV-2
Assay: N2 (US CDC)

RT-PCR was performed with a modified QN Pathogen Mix w/o KCl/NaCl and pH adjusted with acetic acid (see above).

To investigate if the components tested in Example 8 work somehow synergistically, an “extraction solution” comprising an RNase inhibitor (ES) was modified with a combination of a reducing agent (here TCEP) and a non-ionic surfactant (here: Tween20). The composition of the tested “extraction solutions” is shown in Table 3.

TABLE 3

Compositions of the “extraction solution” tested in Example 8
RNase inhibitor 0.5 U
RNase inhibitor 0.5 U + 0.6 mM TCEP+0.5% Tween 20
RNase inhibitor 0.5 U + 0.9 mM TCEP+1% Tween 20

### Results:

The combination of a reducing agent (here: TCEP) and a non-ionic surfactant (here: Tween20) surprisingly showed in combination lower Ct-values. Higher concentrations of the reducing agent and the non-ionic surfactant decreased the threshold by up to one cycle which provides an important improvement by a factor of two. Therefore, it is advantageous to use an extraction composition that comprises (a) an RNase inhibitor, preferably a proteinaceous RNase inhibitor, (b) a non-ionic surfactant and (c) a reducing agent. TCEP is a particularly suitable reducing agent in view of its stability characteristics.

### Example 10: Effect of Heating on PCR Result With and Without “Extraction Solution”

To improve access to the target RNA, surfactants or more complex solutions were added to the sample (see above: “extraction solution”) to support pathogen lysis.

Heating a sample containing a pathogen, especially a virus, preferably at >90° C. has the undisputable benefit of destroying and therefore inactivating the pathogen so the sample can be handled under standard biosafety conditions circumventing the need for time- and cost-intensive preventive measures. Furthermore, an initial heating step can support the pathogen lysis, thereby improving release of the target nucleic acid and therefore will increase sensitivity.

However, previous results with SARS-CoV-2, a RNA virus, have already shown that heating the samples decreases signal intensity (increase in Ct-values) and therefore decreases sensitivity (see above). It is assumed that the observed decreased sensitivity results from the instability of the RNA. E.g. RNA is prone to (auto)hydrolysis especially in the presence of divalent cations like magnesium and calcium. Such cations are often present in the media containing the biological sample, such as HBSS (for the composition see above), which is a major ingredient in commonly used universal transport media, e.g. UTM and VTM.

Even more, heating the biological sample in the presence of a lysis-supporting reagent like a surfactant results in an even higher loss of sensitivity compared to heating alone. This is presumably due to a more complete release of RNases from the contained cells after destruction of intracellular compartments and regulatory mechanisms. All in all, based on current knowledge the inactivation of the target pathogen to be detected especially of an RNA virus is at the cost of target integrity and in consequence of signal intensity.

To have a deeper look into these effects DNA and RNA templates were spiked in UTM medium and UTM medium with 15,000 Jurkat cells per reaction to simulate the effect of RNase release during lysis due to heating.

Samples were either heated for 10 min at 95° C. or not heated and the “extraction solution”, containing a non-ionic surfactant, a disulfide-bond breaking reducing agent (TCEP) and a proteinaceous RNase inhibitor, was added before amplification. Samples without “extraction solution” added were used as reference.
Sample type: UTM with and w/o Jurkat cells
Target: in vitro transcript
Assay: N2 (US CDC)

RT-PCR was performed with a modified QN Pathogen Mix w/o KCl/NaCl and pH adjusted with acetic acid (see above).

### Result:

The initial heating step again resulted in increased Ct-values when the sample was directly added to the (RT-)PCR reaction.

However, very unexpected, the addition of the “extraction solution” according to the present invention, here containing a non-ionic surfactant, a disulfide-bond breaking reducing agent (TCEP) and a proteinaceous RNase inhibitor, had a beneficial effect on the results leading to signal intensities practically identical to those without heating. It was very surprising that the addition of a theoretically lysis-promoting solution (non-ionic surfactant) is able to revert the detrimental effect of heating.

Unexpectedly - “uncoupling” the heating and preparation of the sample to release the target as described herein, i.e. heating the biological sample first in the absence of the “extraction solution” is the crucial point. This initial heating step advantageously leads to pathogen inactivation and the following addition of the “extraction solution” prior to amplification prevents the subsequent degradation of the target nucleic acid due to inhibition of the RNases. Therefore, the “extraction solution” is added after the heating step for pathogen inactivation has been completed.

This embodiment of the present invention advantageously combines the important benefit of pathogen inactivation with the advantage of additional sample lysis without impairing signal intensity in the subsequent amplification reaction.

### Example 11: Effect of Heating Plus “Extraction Solution” on PCR With Real-Life Samples

To demonstrate that this advantageous effect is also present with real-life samples, negative samples from different donors were spiked with targets and analyzed. To increase the load of a potential RNase, 1,200 Jurkat cells per reaction were added.

Samples were heated for 5 and 15 minutes respectively and stored at ambient temperature for 4 hours or 3 days. Non-heated samples and samples processed immediately after heating were used as references. Afterwards, the extraction solution was added and incubated with the sample for 2 minutes at room temperature immediately before the (RT-)PCR reaction according to the standard protocol:
Sample type: nasopharyngeal swabs in UTM with and w/o Jurkat cells
Target: in vitro transcript
Assay: N2 (US CDC)

### Results:

Comparison of the Ct-values after 4 hours and 3 days with the starting point (0 hours) showed only neglectable differences below two cycles and these are within the usual range of fluctuation when working with real-life samples. The decreasing Ct-values of donor 25 after 3 days is presumably an outlier considering all the other data.

These results demonstrated two surprising and completely unexpected effects:
1) The apparently detrimental heating step stabilizes the samples for up to three days and allows long-term storage without the need for freezing the biological sample or addition of a stabilizing agent.
2) The detrimental effect of heating observed in previous experiments can be completely reverted by addition of the “extraction solution” according to the present invention after heating and prior to the amplification reaction. In advantageous embodiments, the “extraction solution” comprises a non-ionic surfactant, a disulfide-bond breaking reducing agent (TCEP) and a proteinaceous RNase inhibitor.

A possible explanation for these beneficial effects may be that the heating step somehow impairs RNases and damages virus particles and cells but does not release the RNA either from the virus particle or from the nucleocapsid protein and so protects the target nucleic acid.

The subsequent addition of the “extraction solution” then releases the target nucleic acid (due to the comprised surfactant) and at the same time inhibits the pre-damaged nucleases (TCEP and RNase inhibitor). Therefore, this embodiment wherein the sample is pre-heated is particularly advantageous.

### Example 12: Crude Samples Contain High Amounts of Salts

After swabbing, the swabs with the patient’s sample were put into a transport medium like UTM (Universal Transport Medium, Copan) or VTM. Even if the exact composition of UTM is not published, it is known that UTM as well as VTM contain HBSS as the major fraction.

TABLE 4

Composition of HBSS (retrieved on Sep. 30, 2020 at https://www.aatbio.com/resources/buffer-preparations-and-recipes/hbss-hanks-balanced-salt-solution)

| | | |
|---|---|---|
| Component | Amount | Concentration |
| NaCl (mw: 58.44 g/mol) | 8 g | 0.14 M |
| KCl (mw: 74.55 g/mol) | 400 mg | 0.005 M |
| CaCl₂ (mw: 110.98 g/mol) | 140 mg | 0.001 M |
| MgSO₄-7H₂O (mw: 246.47 g/mol) | 100 mg | 0.0004 M |
| MgCl₂-6H₂O (mw: 203.303 g/mol) | 100 mg | 0.0005 M |
| Na₂HPO₄-2H₂O (mw: 177.99 g/mol) | 60 mg | 0.0003 M |
| KH₂PO₄ (mw: 136.086 g/mol) | 60 mg | 0.0004 M |
| D-Glucose (Dextrose) (mw: 180.156 g/mol) | 1 g | 0.006 M |
| NaHCO₃ (mw: 84.01 g/mol) | 350 mg | 0.004 M |

As demonstrated in the literature also discussed above, this – for an enzymatic amplification reaction – high ionic strength inhibits the reverse transcriptase as well as the DNA polymerase and leads to a dramatic decrease of sensitivity.

### Example 13: Salt as the Major Inhibitory Factor (I)

To investigate the inhibitory effect of the high salt concentrations on PCR, increasing volumes of UTM and PBS (1, 2, 5 µl) were added to each (RT-)PCR reaction.

### Results:

The results clearly demonstrate the strong inhibitory effect of salts (and other components) on the RT-PCR reaction. Whereas the DNA-polymerase was fine even with 5 µl of UTM or PBS added, the RT shows already a slight increase in Ct-values even with 2 µl added and with 5 µl PBS the amplification was dramatically impaired.

### Example 14: Salt as the Major Inhibitory Factor (II)

Example 14 was set-up to verify the putative inhibitory effect of salt on the PCR reaction. A modified QN reaction buffer according to the present invention was prepared wherein the alkali metal salt concentration was reduced compared to the standard QN reaction buffer. The master mix prepared using this modified QN reaction buffer was compared to the standard QN master mix when increasing amounts of a 0.9% NaCl solution were added to prepare the amplification reaction admixture.

To distinguish effects of the solutions on the reverse transcriptase from effects on the DNA polymerase a DNA template as well as a RNA template were used in each reaction.

### Results:

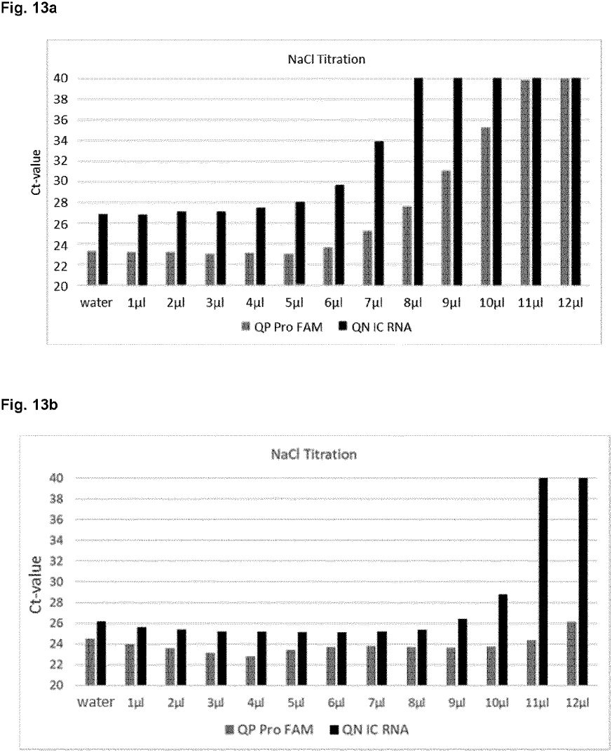

As already shown in Example 13, with the standard QN reaction buffer, a delay in signals (increasing Ct-values) was observed when more than ⅘ µl of a 0.9% NaCl solution were included into the PCR reaction admixture (see FIG. 13a). In contrast, using the modified PCR reaction buffer having a reduced alkali metal salt concentration to set up the master mix, the reaction can cope with up to 9 µl of a 0.9% NaCl solution and - surprisingly - even with no or small amounts added the impact on the results was neglectable (see FIG. 13b). Even more, moderate addition of the salt solution to the reaction resulted in a slight increase in sensitivity (lower Ct-values) under the tested conditions (see FIG. 13b). This beneficial effect was presumably seen because a minimal ionic strength is beneficial to the RT and/or PCR reaction and the original conditions were restored in part due to the addition of the NaCl solution to the PCR reaction admixture that was prepared using the modified PCR reaction buffer with reduced alkali metal salt concentration.

Therefore, when processing biological samples contained in saline media, in particular media that contain alkali metal salts such as sodium chloride and/or potassium chloride, it is beneficial to use a amplification reaction buffer, respectively master mix according to the present invention, wherein the alkali metal salt concentration is reduced to thereby compensate the introduction of alkali metal salts into the amplification reaction admixture due to the medium that contains the biological sample. Therefore, for a direct amplification method (including PCR and RT-PCR) that processes crude samples comprising the biological sample contained in salt-containing media, it is beneficial to use an amplification master mix according to the present invention, wherein the alkali metal salt concentration is reduced compared to standard amplification master mixes. Therefore, in one embodiment, the amplification reaction buffer, respectively the amplification master mix, does not comprise sodium chloride and/or potassium chloride. In a preferred embodiment, the amplification reaction buffer, respectively the amplification master mix, contains neither sodium chloride nor potassium chloride. In a further embodiment, no alkali metal salts are contained in the amplification reaction buffer, respectively the amplification master mix, according to the present invention.

### Example 15: Salt as a Major Inhibitory Component of Crude Samples

Using a modified QN reaction buffer as described in Example 14 (reduced alkali metal salt concentration), the effect of high salt input on a (RT-)PCR reaction was determined by adding increasing amounts of high salt solutions into the reaction mix: 0.9% NaCl solution (see example 14), UTM and PBS. To distinguish effects of the solutions on the RT from effects on the polymerase, a DNA as well as a RNA template was used in each reaction.

### Results:

The results again clearly show the inhibitory effect of the added salt(s) contained in standard transport media on the RT and PCR reaction. However, when using a modified amplification reaction buffer, respectively modified amplification master mix, according to the present invention, wherein the alkali metal salt concentration is reduced compared to the prior art, there is nearly no effect even up to 9 µl of the high salt solutions included into the 20 µl amplification reaction admixture as demonstrated from the PBS and 0.9% NaCl results. Larger volumes resulted in increasing inhibition until the amplification completely failed.

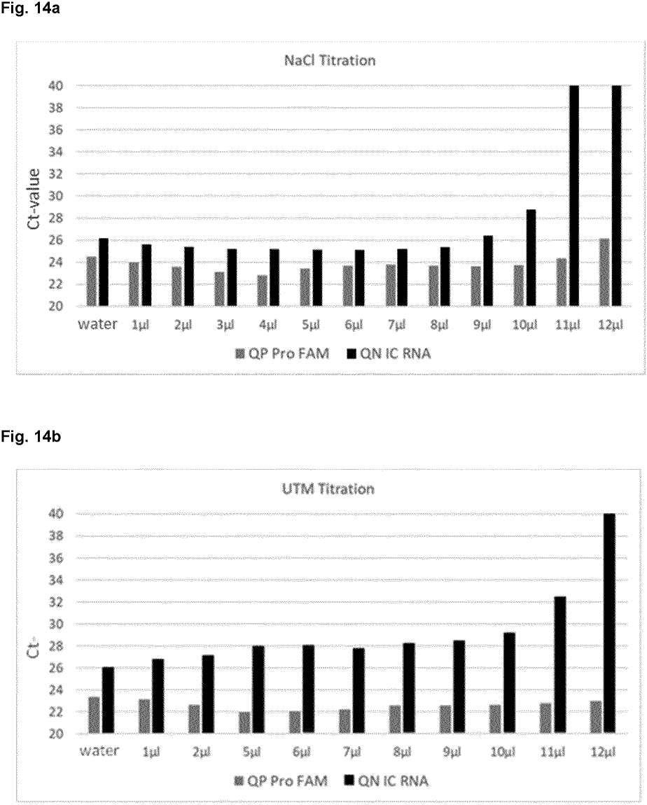

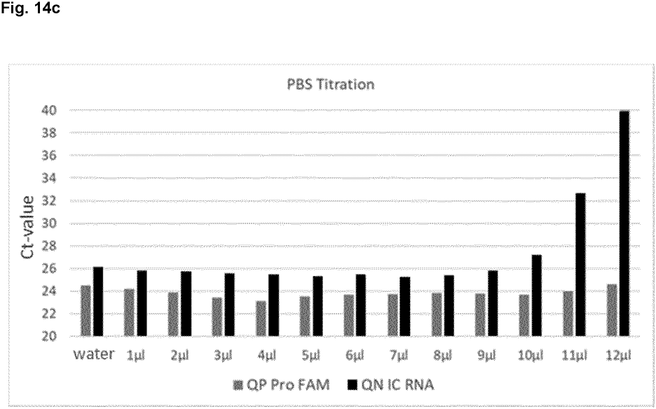

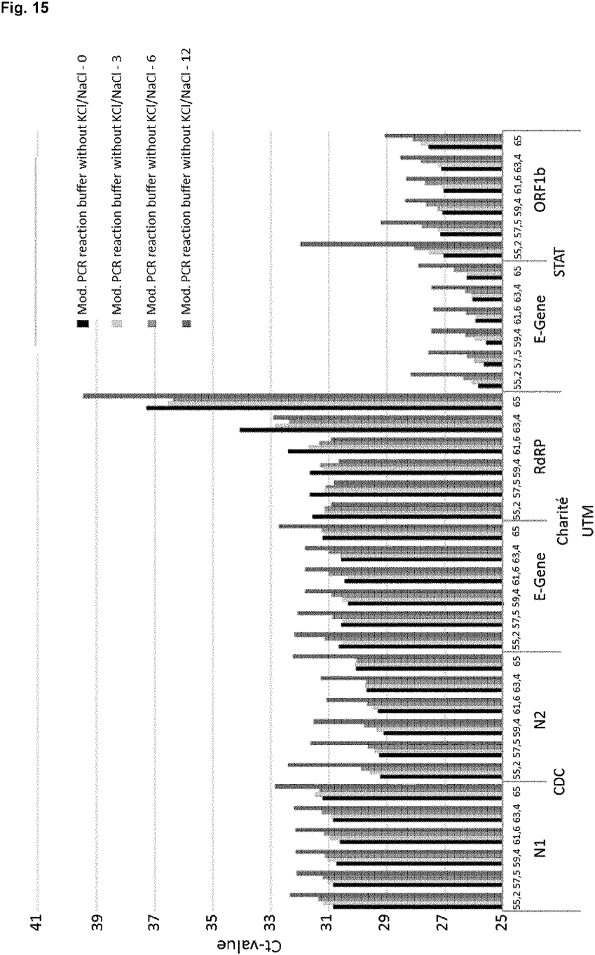

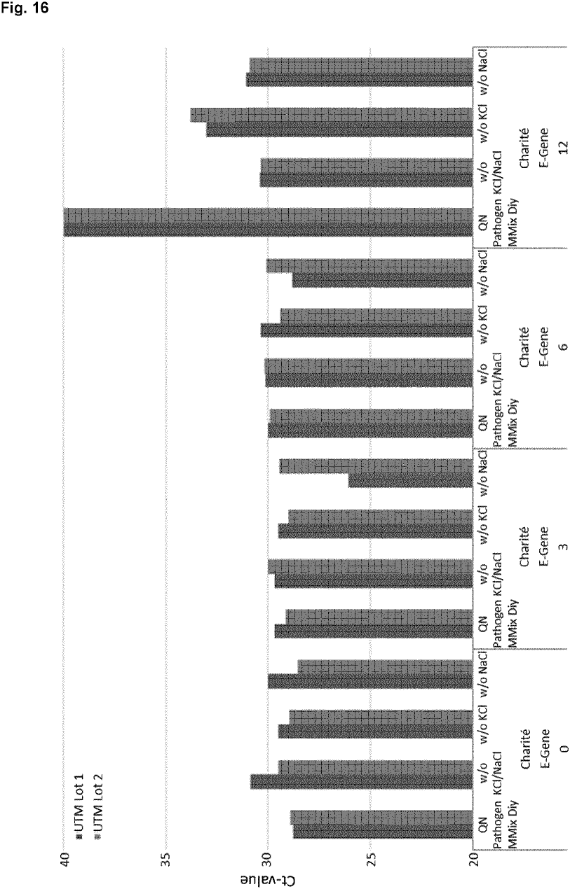

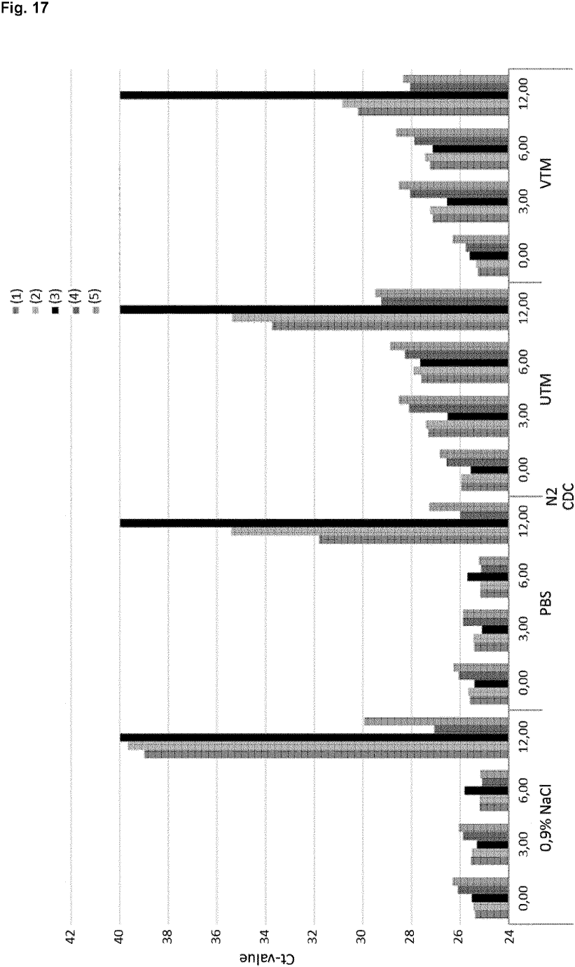

Surprisingly the DNA polymerase was essentially not inhibited as can be seen from FIGS. 14a-14c. On the contrary, a moderate addition (3-6 µl) of the high salt solutions improved the reactions by mimicking the original conditions. The reverse transcriptase was strongly inhibited at higher salt concentrations.

### Example 16: Robustness of a NaCl- and KCl-Depleted PCR Reaction Buffer With Different Sample Input and Primer Annealing Conditions

Sample type: oropharyngeal swab in UTM
Target: in vitro transcripts; 500 copies
Assays: SARS-CoV-2 genes N, RdRP, E, Orf1b

As discussed above, the prior art QN reaction buffer comprises KCl. KCl is a standard component in PCR reaction buffers because it neutralizes the charge present on the backbone of the DNA and helps in the annealing of the primer and stabilizes the primer-template binding. It is considered essential for successful amplification. The previous examples demonstrated that an amplification reaction buffer, respectively amplification master mix, according to the present invention which has a reduced alkali metal salt concentration compared to the standard prior art is beneficial when aiming at performing a direct amplification using the crude biological samples comprised in transport media without prior purification of the nucleic acids.

In Example 16, a modified amplification reaction buffer according to the present invention was prepared without KCl and without NaCl to demonstrate that using such PCR reaction buffer for setting up the amplification master mix can increase the sensitivity of the reverse transcription amplification. The further components corresponded to the QN reaction buffer.

It is well known that the ionic strength of a solution influences the annealing behavior of primers to the template and therefore a temperature gradient was applied to investigate the effect on the new reaction buffer in combination with a crude sample input.

### Results:

The results clearly demonstrate that an amplification reaction buffer without KCl and NaCl dramatically improves the sensitivity of the detection reaction. Up to 6 µl transport medium – mimicking a crude sample – can be applied with a neglectable increase in Ct-values.

When 12 µl of the transport medium were added an increase in Ct-values was observed. However, for the CDC N1 and STAT Orf1b gene this increase is only in the range of 1 Ct value demonstrating the strong tolerance of the NaCl and KCl-depleted PCR reaction buffer against transport media with high ionic strength. Therefore, in a preferred embodiment, the PCR reaction buffer used to prepare the amplification master mix does not comprise NaCl and KCl, and preferably contains no alkali metal salts.

Furthermore, Example 16 shows that the annealing temperature has nearly no effect on the results. This is a great advantage because it allows the use of existing cycling protocols without the need for additional annealing temperature optimization.

### Example 17: Robustness Against Different Lots of Transport Media

Besides HBSS (see above) FCS is another core ingredient in the UTM / VTM transport media. Because FCS is somehow undefined in its composition it is important to have a robust solution which can cope with the variations in the transport media composition due to a varying FCS composition.

Therefore, two different lots of UTM were tested using amplification reaction buffers according to the invention (1) without KCl and NaCl; (2) without KCl (but comprising NaCl) and (3) without NaCl (but comprising KCl) for setting up the amplification master mix. The standard QN pathogen master mix was run in parallel.

### Results:

No significant differences were observed when comparing two different lots of UTM. This demonstrated the surprising robustness of the amplification reaction buffers according to the present invention, wherein the amount of alkali metal salts is reduced and which in embodiments comprise no alkali metal salts.

### Example 18: Effect of Chloride on the Amplification Reaction

The previous data demonstrated the robustness against high loads of solutions with high ionic strength. Nevertheless, when adding larger amounts, a certain inhibition is still observed (see e.g. Example 14).

Common amplification reaction buffers are buffered with Tris and the correct pH is adjusted with HCl introducing additional chloride ions. It was suspected that the reverse transcriptase could be inhibited by chloride ions and it was therefore tested if an “additional robustness” could be achieved by replacing the HCl by a different buffering agent free of chloride ions. For this purpose, a carboxylic acid, here acetic acid, was tested for adjusting the pH.

The high salt solutions in 0.9% NaCl, PBS, UTM, and VTM were added to different amplification master mixes which either contained or did not contain KCl and NaCl. The pH was adjusted by acetic acid or HCl. The set-up is described in the figure legend.

### Results:

It became obvious that the amplification master mix prepared with the PCR reaction buffer (3) cannot cope with the high amounts of salts after addition of 12 µl of the respective solution and completely failed. Most striking, without any alkali metal salt in the PCR reaction buffer (see (4) and (5)) it was possible to get Ct-values well below 30 even with all solutions when 12 µl were added.

Comparison between the acetates (PCR reaction buffers (1) and (2) - first two bars) and chlorides (PCR reaction buffers (1) - (3) - last three bars) also demonstrated that the overall ionic strength is the main cause for inhibition. However, the replacement of HCl by acetic acid led to a significant decrease in Ct-values with NaCl and PBS when 12 µl of the indicated solutions were added. With UTM and VTM this effect is much smaller indicating an inhibitory effect due to other components in this complex media.

### Example 19: Detection of SARS-CoV-2 Targets From Human Samples With an “Extraction Solution” by Direct PCR

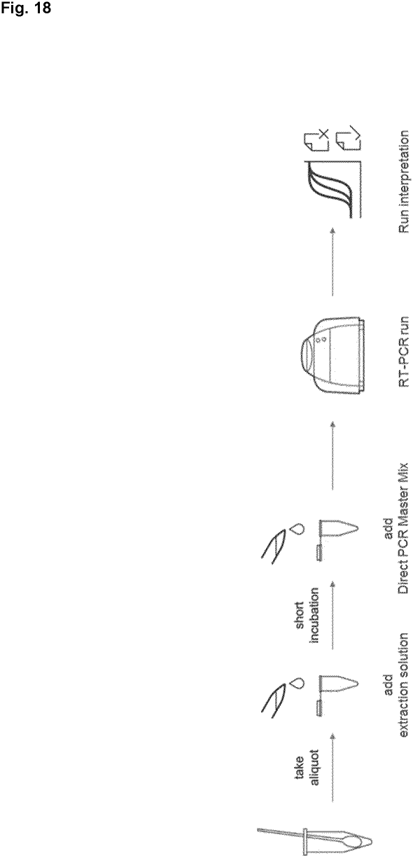

FIG. 18 illustrates an exemplary advantageous workflow of the method according to the invention. This workflow can be advantageously used for the detection of numerous pathogens from human biological samples collected with nasal, nasopharyngeal or oropharyngeal swabs stored in non-fixation transport media like UTM, VTM, PBS or NaCl is presented. As disclosed herein, the method according to the invention is rapid, does not require prior nucleic acid purification and allows the detection of the pathogen target nucleic acids with high sensitivity. It is particularly suited to amplify and thus detect RNA target nucleic acids and can thus be advantageously used for the detection of RNA viruses, such as SARS-CoV-2, in human biological samples.

As is illustrated in FIG. 18, the biological sample (e.g. swab sample) is collected from the subject and placed in medium. This can be a common transport medium or other saline-solution or buffer as described herein. It is also within the scope of the present invention to collect the biological sample (e.g. swab) as dry sample, i.e. without medium, for shipping. In this case, the dry sample is then placed in medium when processing the biological sample. The method thus preferably starts with a biological sample that is contained in medium. The biological sample is agitated in the medium (e.g. vortexing the swab containing sample) and an aliquot of the medium containing the biological sample is then transferred to a reaction vessel (e.g. PCR tube or well). The extraction solution according to the invention is then added to the aliquot of the biological sample contained in medium. It is also within the scope of the present invention to transfer aliquot of the biological sample contained in medium to a reaction vessel that already contains the extraction solution according to the present invention. The resulting admixture is incubated to prepare the biological sample for amplification. As disclosed herein, the incubation time may be short, thereby supporting that the method according to the present invention can be performed rapidly. Afterwards, the prepared biological sample is contacted with the components necessary for performing the amplification reaction, which is a reverse transcription amplification reaction in case of RNA targets. While the prepared biological sample may be transferred to a new reaction vessel comprising the amplification reagents, it is a particular advantage of the present invention that the method can be performed in a single reaction vessel (“one pot”). Therefore, in a preferred embodiment, the components necessary for performing the amplification reaction are added to the same reaction vessel containing the incubated and thus prepared biological sample. To further ease the handling and reduce pipetting errors, all components necessary for the amplification reaction may advantageously be included in a direct PCR master mix that contains besides the template all components used for the amplification reaction (including the polymerase(s), nucleotides, primers, probes, potential IC controls etc.). The direct PCR master mix may then be contacted with the incubated admixture and thus the prepared biological sample. The so prepared amplification reaction admixture is then ready for performing the amplification reaction. As is illustrated in FIG. 18, the amplification reaction is in a core embodiment of the present invention a RT-PCR. Advantageously, the reverse transcription and PCR amplification may take place in a single tube.

The following procedure provides a detailed exemplary workflow according to the present invention for the direct detection of SARS-CoV-2 targets from human samples with an “extraction solution” by a PCR system:

1. Prepare the amplification reaction admixture by mixing the Direct PCR Master Mix, the sample and the “extraction solution” of the invention (comprising a proteinaceous RNase inhibitor, a non-ionic surfactant and a reducing agent) according to Table 5 and described in further detail below:

TABLE 5

Reaction mix setup

| | Component | Volume added to PCR tube / well of a PCR plate [µl] | Final concentration |
|---|---|---|---|
| Direct PCR Master Mix | Master Mix, 4x | 5 | 1x |
| | SARS-CoV-2 Assay, 20x | 1 | 1x |
| | RNA IC Template + Assay, 10x | 2 | 1x |
| | Human Sampling IC Assay, 20x | 1 | 1x |
| | Rox reference dye (ABI instruments only) | 1 / 0.1* | 1x |
| | RNase-free water | Fill up to 10 | - |
| Prepared sample (steps 3 and 4) | Sample | 8 | - |
| | Extraction solution | 2 | 1x |
| Total reaction volume | | 20 | - |

*To be used as a 200x concentrate for low ROX dye cyclers and as a 20x concentrate for high ROX dye cyclers

2. Vortex the swab-containing sample vigorously.

### Steps 3 and 4 - Variant A

3a. Dispense 2 µl of the “extraction solution” according to the invention in each PCR tube or well of a PCR plate.

4a. Add 8 µl of the swab sample to the individual PCR tube or well containing the “extraction solution” (steps 3a and 4a may also be reversed). Mix by pipetting up and down at least two times for thorough contacting.

### Steps 3 and 4 - Variant B

3b. Dispense 8 µl of the sample in each PCR tube or well of a PCR plate and heat the sample at 85° C.-100° C. for 5 to 15 min (e.g. 95° C., 5 min). As shown in the examples above, this optional heating step prior to contacting the biological sample with the “extraction solution” allows to inactivate viruses and thereby increases the safety when handling biological samples potentially contaminated with viruses.

4b. Add 2 µl of the “extraction solution” according to the invention to the individual PCR tube or well containing the heat inactivated biological sample. Mix by pipetting up and down at least two times for thorough contacting.

5. Incubate at room temperature for 2 min.

Note: If multiple samples are processed in parallel, the incubation time should start after adding the last sample to the “extraction solution”.

6. Add 10 µl of the Direct PCR Master Mix prepared in step 1.

7. Seal the plate/tube thoroughly to prevent cross contamination. In case an adhesive film is used, make sure to apply pressure uniformly across the entire plate, in order to obtain a tight seal across individual wells. Mix gently by vortexing for 10-30 seconds with medium pressure. Place the plate in different positions while vortexing to ensure an equal contact with the vortex platform. Centrifuge the plate/tube briefly to collect liquid at the bottom of the plate/tube.

8. Program the real-time cycler, e.g. according to Table 6.

Note: Data acquisition should be performed during the annealing/extension step.

TABLE 6

Cycling conditions

| Step | Time | Temperature [°C] | Ramp rate |
|---|---|---|---|
| RT-step | 10 min | 50 | maximal / fast mode |
| PCR initial heat activation | 2 min | 95 | maximal / fast mode |
| 2-step cycling | | | |
| Denaturation | 5 sec | 95 | maximal / fast mode |
| Combined annealing/extension | 30 sec | 58 | maximal / fast mode |
| Number of cycles | 40 | | |

9. Place the tubes or plates in the real-time cycler and start the cycling program.

Table 7 illustrates possible outcomes of the direct detection method of SARS-CoV-2 targets from human biological samples (e.g. swab samples) prepared for direct amplification using an “extraction solution” according to the present invention, also indicating the status of common controls used in a pathogen assay.

TABLE 7

Possible outcomes of a direct detection method of SARS-CoV-2 targets from human samples

| | | | | |
|---|---|---|---|---|
| Viral RNA Assay | Internal Control | Sampling Control | Status | Result |
| + | + | + | valid | positive |
| + | + | - | valid | positive |
| + | - | - | valid | positive |
| + | - | + | valid | positive |
| - | + | + | valid | negative, not detected |
| - | + | - | inconclusive | repeat test using a new sample |
| - | - | + | PCR inhibited | repeat test using a lower input volume |
| - | - | - | PCR inhibited | repeat test using a lower input volume |

### Example 20: Workflows for Viral Detection From Different Sample Materials

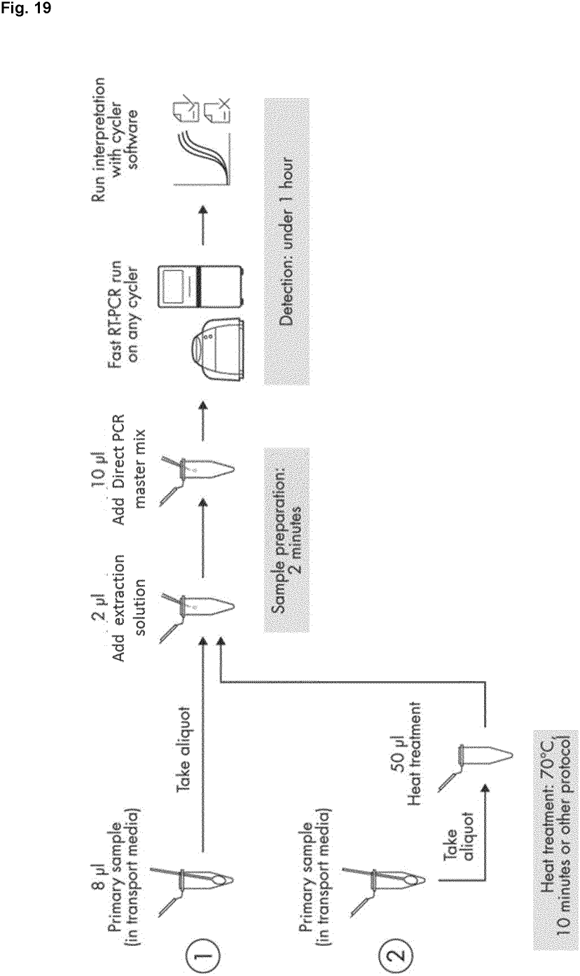

The invention outlined in the above examples can be utilized in convenient workflows. The protocol described schematically in FIG. 19 is based on buffer conditions and protocol steps that were chosen based on the previous examples (Examples 1 - 19). The workflows allow the detection of viruses in transport media using swab samples.

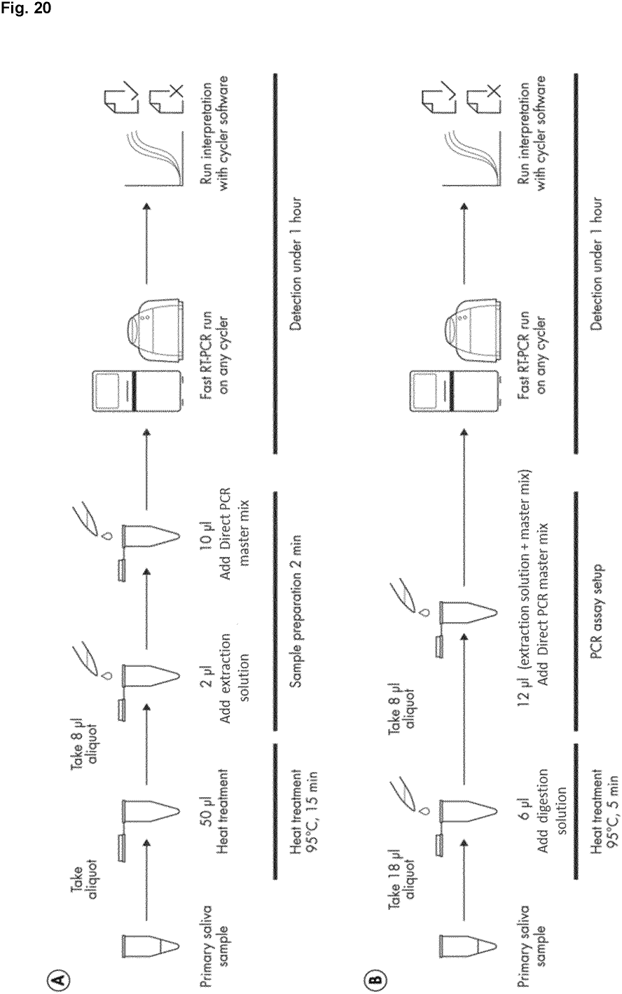

The chemistry and protocol were further developed and optimized for use with oral samples, such as in particular saliva or gargle samples, as shown schematically in FIG. 20. As shown in the following examples, the preparation of such samples for amplification based detection can be further improved by contacting the sample with a digestion solution that comprises a proteolytic enzyme and a reducing agent. The digestion solution can be applied to the sample e.g. in a ratio of 1:4 to 1:1. In embodiments it is applied in a ratio of 1:3 or 1:2. The sample in contact with the digestion solution is heated. Heating is preferably at a temperature of at least 90° C., or at least 95° C. Short heating times are sufficient, e.g. 5 min -10 min, preferably 5 min. The proteolytic enzyme is in advantageous embodiments a protease, such as preferably proteinase K. As reducing agent, TCEP may be used. In the following examples, a digestion solution comprising proteinase K and TCEP was used, unless indicated differently.

For oral samples, such as in particular saliva and gargle samples, two core protocol options are shown in the following examples: 1) use of the digestion solution to stabilize and lyse the sample in combination with heating for 5 min at 95° C. or 2) heating only for 15 min at 95° C. For further details, please refer to the following examples.

### Example 21: Multiplex Detection of Various Pathogens in Different Transport Media

To reduce experimental effort and increase diagnostic speed, parallel detection of multiple targets in one amplification reaction is desirable. For instance, this may include detection of different amplicons of the same pathogen and/or the parallel detection of different pathogens.

To demonstrate the suitability of the present invention for multiplex detection, several inactivated respiratory viruses were added to different transport media and detected in parallel using assays for the different targets according to the workflow shown in FIG. 19.

Sample types: sampled healthy donor in different transport media
eSwab
physiological NaCl-solution
PBS
UTM
VTM

Targets: inactivated viruses
SARS-CoV-2 (SC2)
Flu A
Flu B
RSV

Assay: N1 / N2 (US CDC)

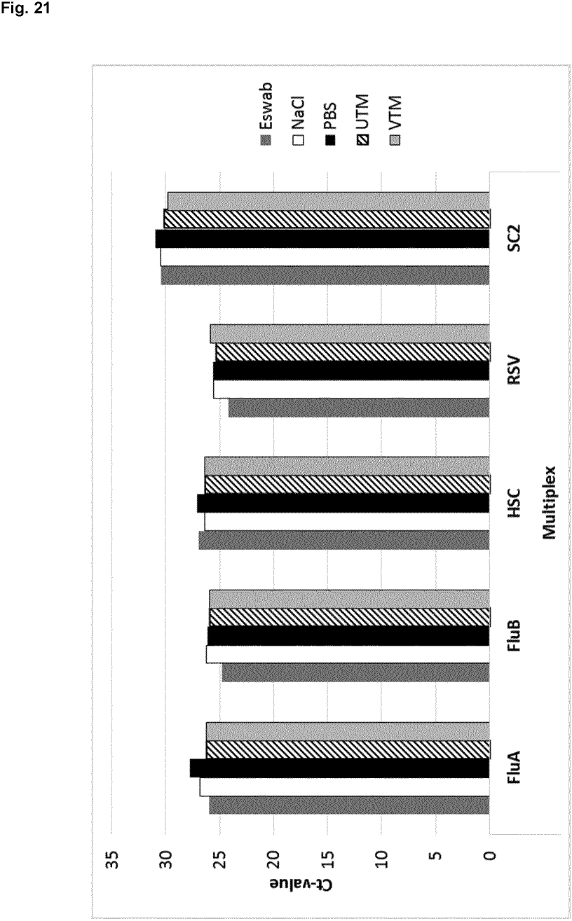

RT-PCR was performed with the modified QN Pathogen Master Mix without NaCl / KCl, pH adjusted with acetic acid as described above. All targets were amplified in parallel. The individual signals for each target are given in FIG. 21.

### Results:

The results clearly show that it is possible to detect a set of different respiratory viruses in parallel. The results also demonstrate that the workflow of the present invention can be applied to every common transport medium without significant effects on the sensitivity of the system represented by the homogeneous Ct-values.

### Example 22: Genotyping of Virus Variants

During the COVID-19 pandemic, a number of new variants of SARS-CoV-2 have evolved, some of which are more contagious than the original wild-type strain, requiring stricter distance and quarantine requirements. Therefore, it is important to know not only whether a person is infected, but also which strain the person is infected with. To avoid expensive and time-consuming sequencing analysis, PCR detection and identification of the different viral variants is essential.

This example demonstrates the successful detection of two different viral variants of SARS-CoV-2 (T478K and E484K) and clear differentiation from the wild-type strain. Dilution series using a standard of inactivated virus following the workflow shown in FIG. 19 with a primer/probe combination specific for T478K and E484K, respectively, is shown. A wild-type transcript (10^{^}7 copies) was used as reference.
Sample type: UTM
Target: in vitro transcripts

### Results:

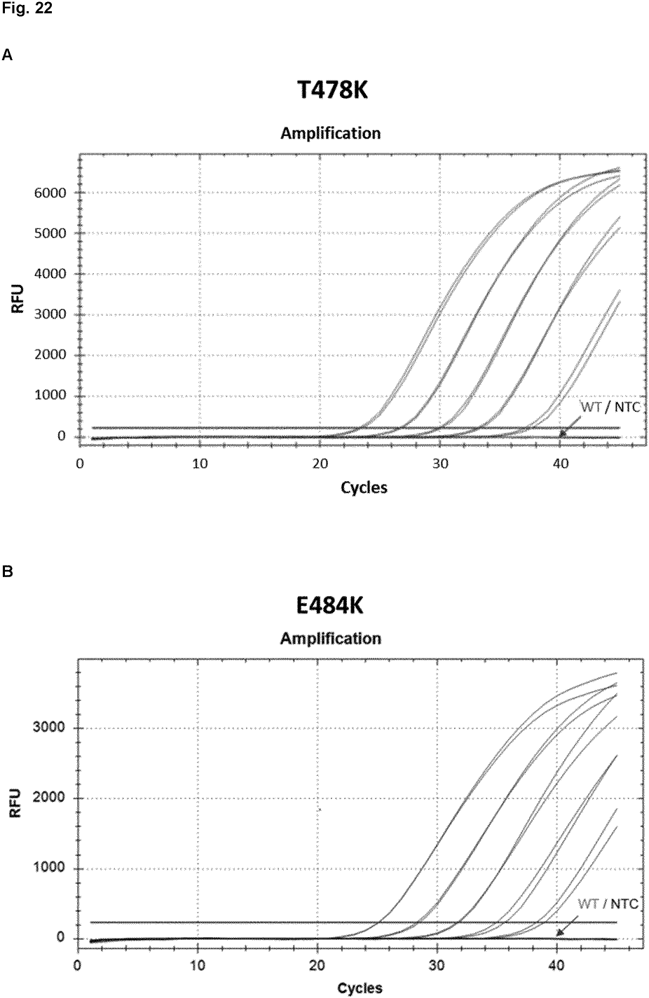

Both variants could be detected according to the different dilutions whereas no wild type signal was detectable (FIG. 22, baseline, identical to the NTC).

This clearly shows that different virus variations could be detected and discriminated from the wild type (and therefore other virus variants) and thus the invention allows rapid genotyping and identification of the different virus strains.

### Example 23: Effect of TCEP on Saliva Samples

For detection of viral nucleic acids comprised in saliva samples, an additional step to lyse and stabilize the viral sample was included to further improve the processing of this sample type since saliva comprises a higher content of enzymes and other proteins compared to swab samples.

As already outlined in Examples 7 and 8 above, addition of a reducing agent like TCEP, DTT, or beta-mercaptoethanol has been proven to have a positive effect on RNA stability due to their ability to disturb (disulfide bond containing) nucleases and no negative effect was observed in amplification reactions within the tested concentration ranges.

However, to determine to which extent additional reducing agents can be added to the amplification reaction a broader concentration range was tested.
Sample type: UTM
Target: in vitro transcript SARS-CoV-2 with sampling and internal control
Assay: N1 / N2 (US CDC)

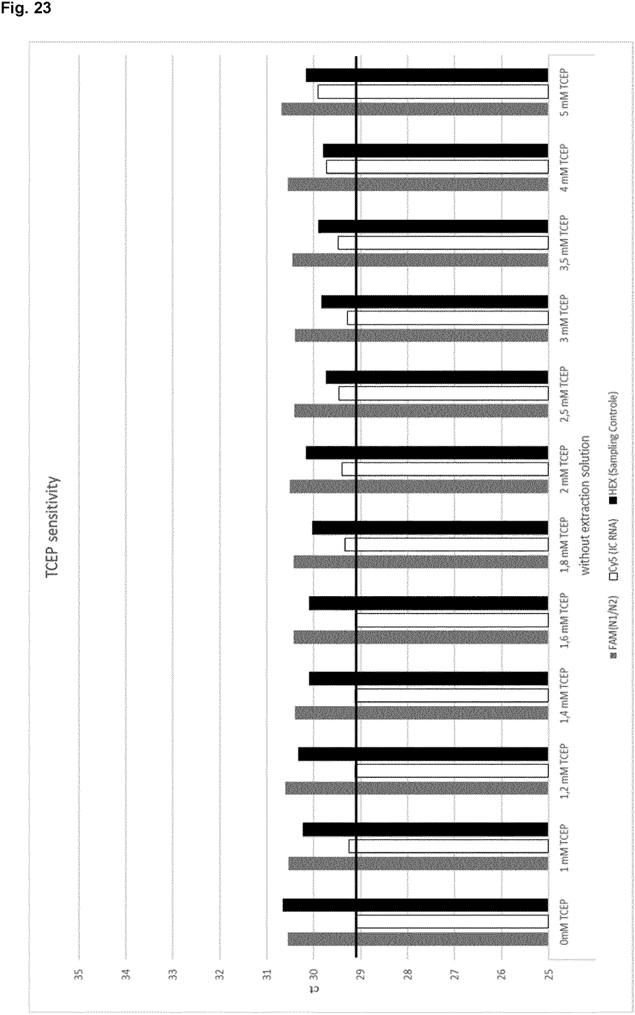

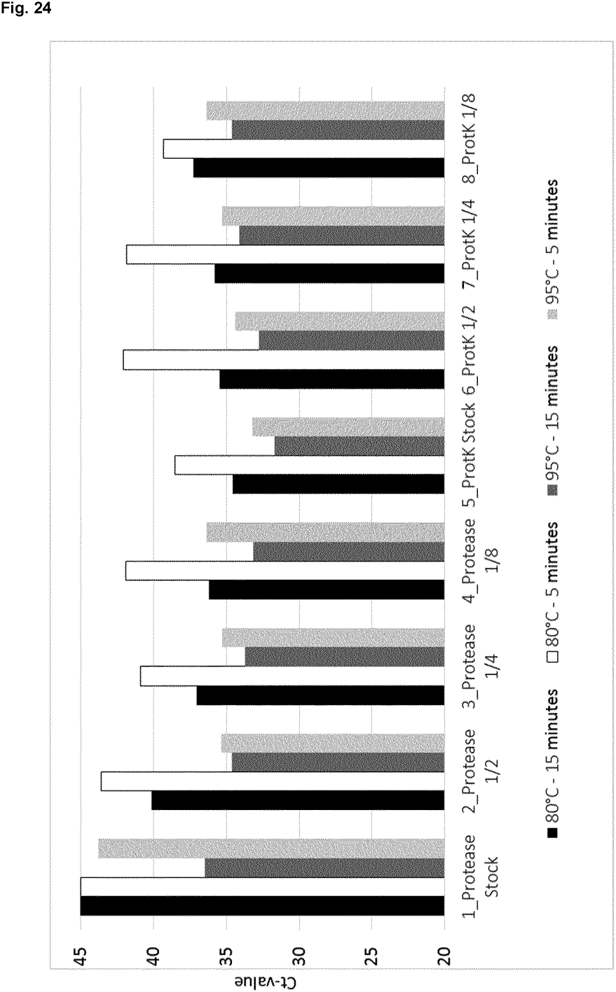

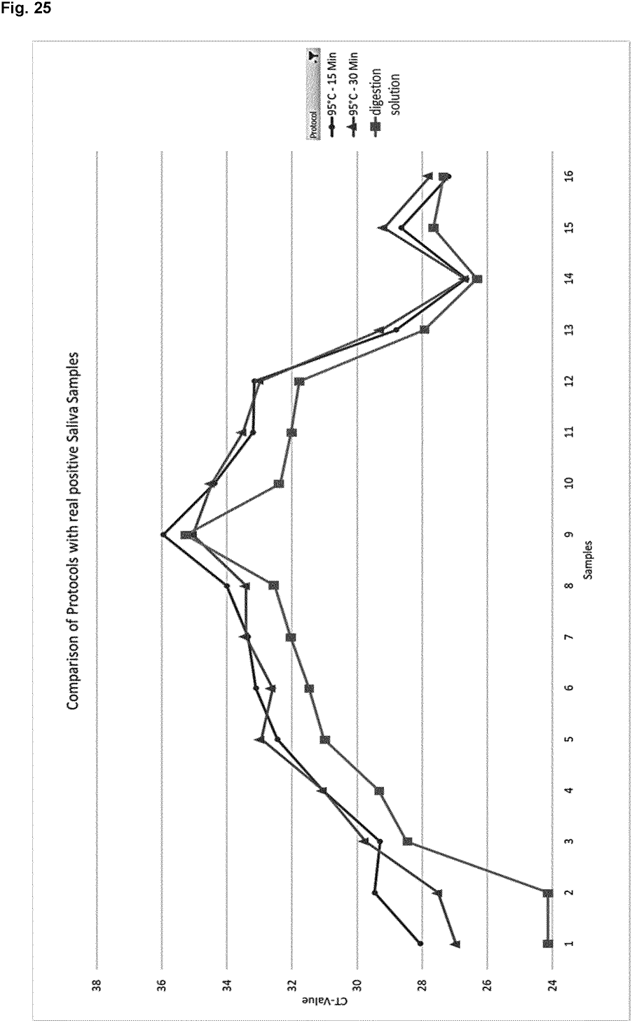

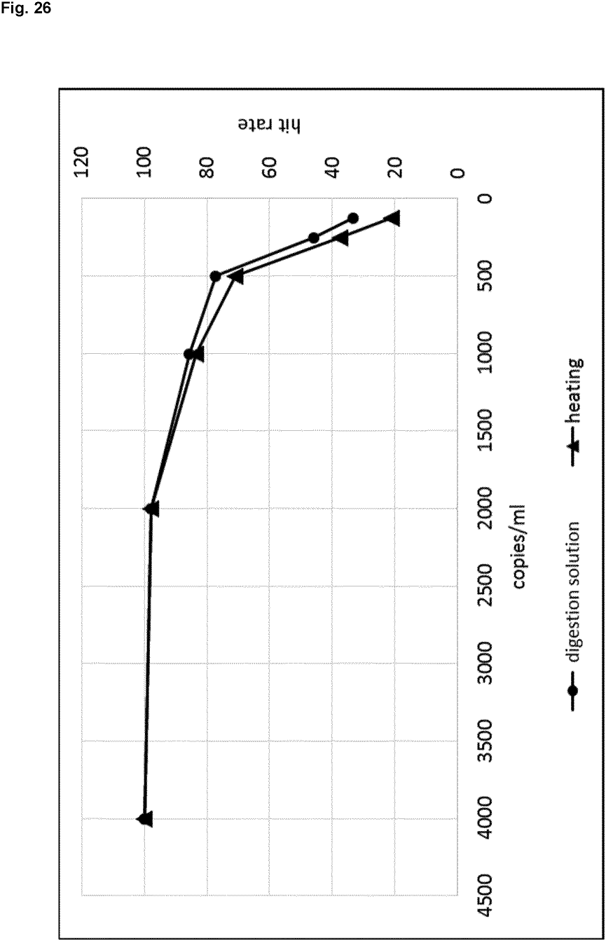

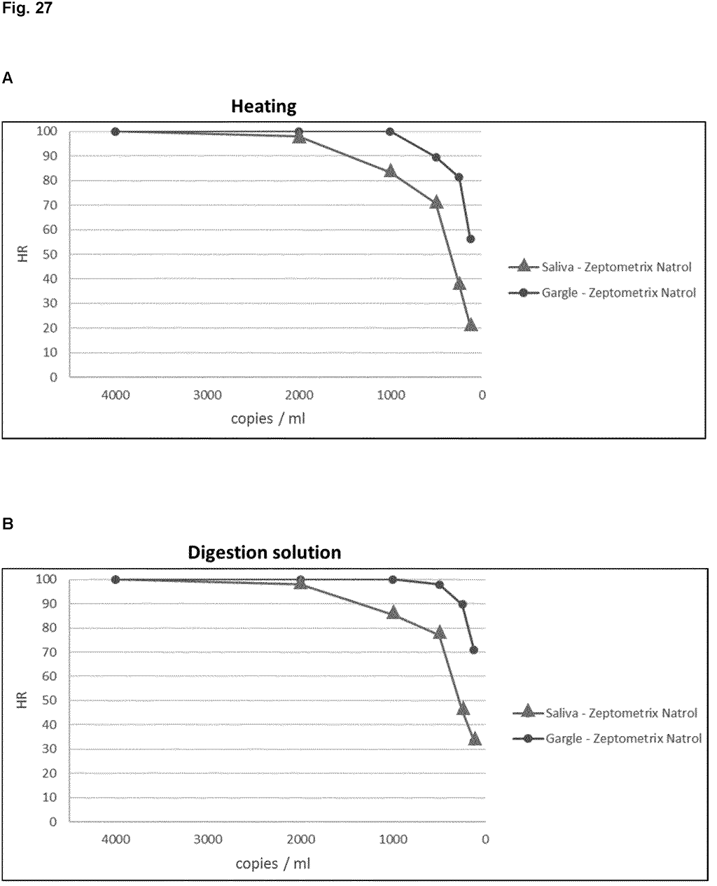

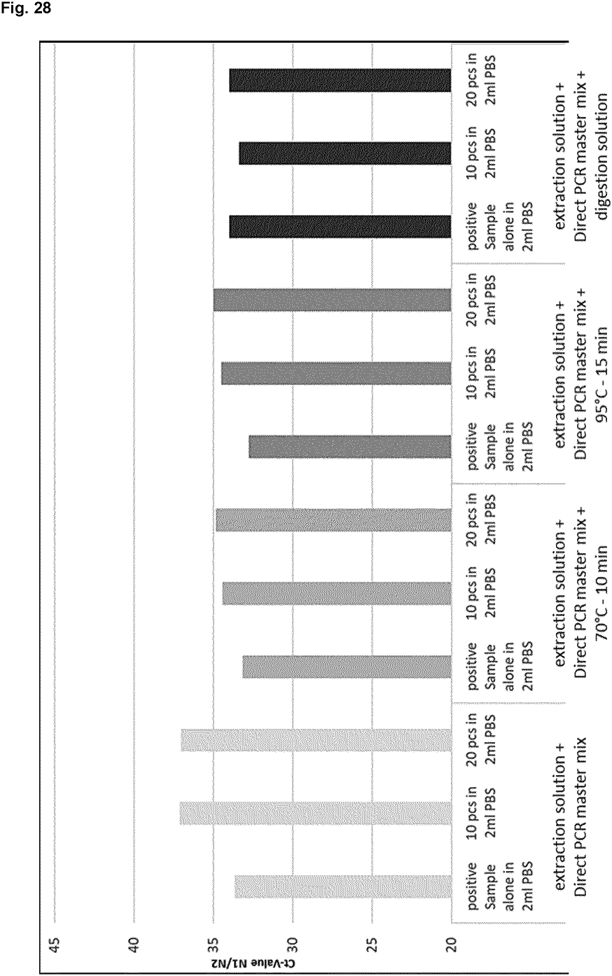

RT-PCR was performed without the extraction buffer/solution step. Increasing amounts of TCEP were added from 0 to 5 mM (see FIG. 23).

### Results:

The experiment showed that a final TCEP concentration in the RT-PCR reaction up to 1.6 mM has no effect on the amplification reaction and only a moderate effect up to a concentration of 3 mM.

Thus, an additional solution containing a reducing reagent such as TCEP can be included in the preparation of the sample without compromising the amplification as long as the total amount of the reducing agent in the amplification reaction does not disturb the amplification reaction. As shown, for TCEP, the total amount of TCEP in the amplification reaction is preferably below 2 mM.

### Example 24: Effect of Proteinases/TCEP on Saliva Samples

Addition of a reducing agent improves the (RNA) stability of the sample. For better lysis of protein rich samples, such as saliva samples, a digestion step with a proteolytic enzyme, such as a prote(in)ase, was included. To determine the effects of an additional protein digestion, different amounts of QIAGEN Proteinase K (Cat. No. / ID: 19131) and QIAGEN Protease (Cat. No. / ID: 19157) were added in the presence of TCEP and tested in combination with a heating step (https://www.qiagen.com/us/products/discovery-and-translational-research/lab-essentials/enzymes/qiaqen-protease-and-proteinase-k/).

24 µl saliva were added to 4 µl of a Prote(in)ase / TCEP / water mixture (see Table 8) and heated for 5 min or 15 min at 80° C. or 95° C., respectively. The internal control to be sensitive to inhibition was therefore chosen as the target control.

TABLE 8

Composition of the Proteinase / TCEP / water mixtures

| | Protease / ProtK [µl] | Water [µl] | TCEP [µl] (16.8 mM) |
|---|---|---|---|
| Protease Stock | 20 | 0 | 6.7 |
| Protease ½ | 10 | 10 | |
| Protease ¼ | 5 | 15 | |
| Protease ⅛ | 2.5 | 17.5 | |
| ProtK Stock | 20 | 0 | |
| ProtK ½ | 10 | 10 | |
| ProtK ¼ | 5 | 15 | |
| ProtK ⅛ | 2.5 | 17.5 | |

Sample type: saliva
Target: in vitro transcript
Assay: Internal control

### Results:

The comparison of QIAGEN Protease and Proteinase K at different concentrations showed higher Ct-values for the internal control when using the QIAGEN Protease compared to the Proteinase K. With Proteinase K, better results were thus obtained compared to QIAGEN Protease. The Ct-values decrease and thus improve with reduced amounts of QIAGEN Protease. Only when the subsequent heating step was shortened to 5 min at 80° C. the effect was less clear which could be explained by incomplete inactivation of the enzymes. The Ct-values were lower and thus improved with higher heating temperatures (95° C.). As demonstrated, different heating periods (5 min-15min) can be used. The use of short heating times, such as 5-10 min, preferably 5 min, is advantageous in view of the reduced preparation time.

### Example 25: Comparison of Different Protocols for Saliva

As shown below, it is possible to inactivate / lyse more difficult to process oral samples, such as saliva samples, either with a digestion solution and 5 min heating or by a heating step only prior to contacting the so pre-lysed/inactivated sample with an extraction solution as disclosed herein. For this, saliva samples from 16 SARS-CoV-2 positive donors were used to compare the digestion solution-assisted 5 min 95° C. heating step with heating only for 15 min and 30 min at 95° C., respectively.
Sample type: UTM
Target: saliva samples, positive for SARS-CoV-2
Assay: N1 / N2 (US CDC)

### Results:

All positive donors were also correctly diagnosed positive with the PCR protocols according to the invention. In almost all cases, the digestion solution comprising a proteolytic enzyme and a reducing agent (such as proteinase K and TCEP) in combination with a short heating step showed the most sensitive results compared to heating alone. This experiment also showed that a longer heating step - 30 min vs. 15 min - did not further improve the results.

### Example 26: Improved Level of Detection (LoD) for Saliva Samples

Sampling with nasal and oropharyngeal swabs is often considered uncomfortable for the subject and requires trained personnel for proper sampling. Therefore, other types of sampling are becoming more prevalent. For example, these include saliva (spitting), gargling with saline or the so-called “lollipop” test, in which the test person simply takes a swab in the mouth and sucks for some time until the swab is saturated.

As noted, the use of saliva differs from swabs due to the high content of enzymes and other proteins compared to swab specimens in the transport medium. The present disclosure provides two workflows - as illustrated in FIG. 20 - that establish a simple and straightforward workflow analogous to the workflow for swabs in transport medium:
1) Including a heat inactivation / lysis step to inactivate and lyse the virus and at the same time inactivate enzymes which interfere with the subsequent amplification reaction (FIG. 20(A))
2) Adding a digestion solution comprising a proteolytic enzyme in combination with a short heating step (FIG. 20(B)).

State of the art for saliva samples is the so called “Yale protocol”, published by the Yale School of Public Health which got an Emergency Use Authorization (EUA). According to this protocol a LoD between 3 to 12 copies/µl (3,000 to 12,000 copies/ml) was determined (Yale School of Public Health, Department of Epidemiology of Microbial Diseases SalivaDirect assay EUA Summary — Updated Apr. 9, 2021; page 8/9).

We tested a RT-PCR reaction according to the invention with the digestion solution comprising a proteolytic enzyme and a reducing agent (here: proteinase K and TCEP) / 5 min 95° C. (“digestion solution”) or alternatively with a 15 min heating step (“heating”) following the workflow described in FIG. 20 to determine the LoD. LoD was calculated by the 95% confidence interval based on the lowest detectable copy number.

Hit rate is based on 48 replicates for each protocol
Sample type: saliva
Target: Zeptometrix Natrol (Zeptrometrix, Buffalo NY, USA)
Assay: N1 / N2 (US CDC)

### Results:

Based on the results above, a level of detection was calculated for both protocols:
a) Heating: 2,112 copies/ml
b) Digestion solution: 2,038 copies/ml

Thus, a LoD of about 2,000 copies/ml could be achieved which is about 1,000 copies/ml more sensitive than the current state of the art, the “Yale protocol”, when using a protocol according to the invention. This reduces the level of detection by about 1,000 copies/ml compared to the LoD given for the “Yale protocol” demonstrating a significant improvement compared to the state of the art protocols.

### Example 27: Other Sample Types — Saliva and Gargle Samples

These protocols according to the invention, which improve the processing of saliva samples as shown in the above examples, can also be used for other sample types. In this experiment we compared saliva and gargle samples with the two protocol variations described in FIGS. 20 (A) and (B).
Sample type: Saliva and gargle samples
Target: Zeptometrix Natrol (Zeptrometrix, Buffalo NY, USA)
Assay: N1 / N2 (US CDC)

A Natrol standard dilution series was added to samples from 24 donors (4,000, 2,000, 1,000, 500, 250, 125 copies/ml each) and analyzed in duplicates.

### Results:

Based on the results above, a level of detection was calculated for both protocols:
c) Heating:
   a. Saliva: 2,122 copies/ml
   b. Gargle: 1,302 copies/ml
d) Digestion solution:
   a. Saliva: 2,038 copies/ml
   b. Gargle: 916 copies/ml

Again, for saliva a LoD of about 2,000 copies/ml could be achieved which is about 1,000 copies/ml more sensitive than the current state of the art, the “Yale protocol”. For gargle samples the LoD was even better especially when using the digestion solution in combination with 5 min 95° C. (<1,000 copies/ml).

### Example 28: Other Sample Types — Lollipop Test

These protocols according to the invention, which improve the processing of saliva and gargle samples as shown in the above examples, can also be used for so-called “lollipop tests”. This means that a swab is used like a lollipop and soaked with saliva before being placed in transport solution, e.g. PBS, for further detection. Because it is simple and convenient, this method of sampling has found wide acceptance in schools and kindergartens, where multiple “lollipops” have often been placed in a tube for simultaneous testing to reduce the amount of testing required. If a pool is positive, the members of the pool are tested again individually.

Four different protocols were tested:
1) extraction solution + Direct PCR master mix according to the invention (no heat treatment); see FIG. 19 above
2) Heating: 70° C., 10 mins
3) Heating: 95° C., 15 mins; see FIG. 20(A) above
4) Digestion solution according to the invention; see FIG. 20(B) above

Sample type: lollipop swabs in PBS, pool with 10 or 20 individuals; one known positive swab in each pool
Target: SARS-CoV-2
Assay: N1 / N2 (US CDC)

### Results:

The results demonstrate that all protocol variants correctly gave a positive result for each pool. Whereas “extraction solution + Direct PCR master mix” alone and the heating steps showed a slight increase in Ct-values and therefore a slight loss in sensitivity, the addition of the digestion solution to the workflow comprising the extraction solution + Direct PCR master mix according to the invention led to identical results for the positive donor and the different pools.

42. A method for obtaining a prepared biological sample for amplification based detection of at least one target nucleic acid present in the biological sample without prior target nucleic acid purification, the method comprising:
contacting the biological sample with an extraction composition comprising
(a) at least one surfactant,
(b) at least one nuclease inhibitor, and
(c) optionally at least one reducing agent,
to obtain an admixture comprising the biological sample in contact with the extraction composition, and
incubating the admixture comprising the biological sample in contact with the extraction composition to provide the prepared biological sample for amplification based detection of the target nucleic acid.

43. The method according to claim 42, wherein the surfactant is a non-ionic surfactant,
wherein the non-ionic surfactant is a polyoxyethylene-based non-ionic surfactant,
optionally, wherein the extraction composition comprises a polyoxyethylene fatty acid ester as the non-ionic surfactant that comprises
a fatty acid derived from laureate, palmitate, stearate and oleate, and
a polyoxyethylene component containing from 4 to 100 (CH₂CH₂O) units.

44. The method according to claim 42, wherein the at least one nuclease inhibitor is a proteinaceous RNase inhibitor.

45. The method according to claim 42, wherein the extraction composition comprises (c) the reducing agent, and wherein the reducing agent is selected from Tris(carboxyethyl)phosphine (TCEP), Dithiothreitol (DTT), N-acetyl cysteine, THPP (Tris(hydroxypropyl)phosphine), 1-thioglycerol and beta-mercaptoethanol.

46. The method according to claim 42, wherein the extraction composition is an extraction solution that is selected from the following embodiments (i) to (v):
(i) the extraction solution comprises
(a) at least one non-ionic surfactant,
(b) at least one proteinaceous RNase inhibitor, and
(c) at least one reducing agent;
(ii) the extraction solution comprises
(a) at least one polyoxyethylene-based non-ionic surfactant,
(b) at least one proteinaceous RNase inhibitor, and
(c) at least one reducing agent selected from Tris(carboxyethyl)phosphine (TCEP), Dithiothreitol (DTT), N-acetyl cysteine, THPP (Tris(hydroxypropyl)phosphine) and 1-thioglycerol;
(iii) active ingredients of the extraction solution consist essentially of
(a) a non-ionic surfactant,
(b) a proteinaceous RNase inhibitor, and
(c) a reducing agent;
(iv) the extraction solution comprises
(a) at least one polysorbate,
(b) at least one proteinaceous RNase inhibitor, and
(c) Tris(carboxyethyl)phosphine (TCEP),
(v) active ingredients of the extraction solution consist essentially of
(a) a polysorbate,
(b) a proteinaceous RNase inhibitor, and
(c) Tris(carboxyethyl)phosphine (TCEP).

47. The method according to claim 42, wherein the method comprises heating the biological sample in the absence of the extraction composition at a temperature suitable to inactivate pathogens prior to contacting the pathogen heat-inactivated biological sample with the extraction composition, optionally, wherein heating for inactivating pathogens comprised in the biological sample prior to contacting the heat-inactivated biological sample with the extraction composition comprises heating the biological sample to ≥ 80° C.

48. The method according to claim 42, having at least one of the following characteristics (aa) to (ff):
(aa) the at least one target nucleic acid is selected from RNA and/or DNA;
(bb) the at least one target nucleic acid is a pathogen-derived nucleic acid, wherein the pathogen is selected from the group consisting of a virus, a bacterium, a protozoan, a viroid and a fungus;
(cc) the at least one target nucleic acid is a viral nucleic acid derived from a virus;
(dd) the at least one target nucleic acid is a viral RNA derived from an RNA virus;
(ee) the at least one target nucleic acid is derived from a coronavirus;
(ff) the target nucleic acid is provided by two or more target nucleic acids derived from the same pathogen.

49. The method according to claim 48, wherein the one or more target nucleic acids are derived from SARS-CoV-2, optionally wherein the target nucleic acid sequences are derived from the SARS-CoV-2 genes N, N1, N2, RdRP, E and/or Orf1b.

50. The method according to claim 42, wherein the biological sample is a respiratory specimen.

51. The method according to claim 42, wherein the biological sample is contained in a medium, optionally wherein the medium has at least one of the following characteristics:
(aa) it comprises Hank’s balanced salt solution;
(bb) it is a salt containing solution;
(cc) it is a physiological salt solution;
(dd) it is a solution comprising 0.7% to 1.2% (w/v) alkali metal salts;
(ee) it is a 0.9% (w/v) sodium chloride solution;
(ff) it is a phosphate buffer, optionally a PBS buffer;
(gg) the medium comprises or consists of Hank’s balanced salt solution, Universal Transport Medium (UTM), Viral Transport Medium (VTM) or has a total salt concentration in a range +/- 30% compared to one or more of (aa) to (ff).

52. The method according to claim 42, wherein after incubation of the admixture comprising the biological sample, the extraction composition and optionally medium, the method further comprises subjecting at least an aliquot or all of the prepared biological sample to an enzymatic reaction, and performing the enzymatic reaction.

53. The method according to claim 52, wherein the enzymatic reaction comprises an amplification reaction, optionally wherein the amplification reaction has one or more of the following characteristics:
(i) it is a reverse transcription amplification reaction;
(ii) it is a reverse transcription PCR;
(iii) it is an isothermal amplification reaction;
(iv) it is a polymerase chain reaction (PCR);
(v) it is a quantitative PCR;
(vi) it is a quantitative reverse transcription PCR; and
(vii) it is a digital PCR.

54. The method according to claim 52, wherein the prepared biological sample that is subjected to the enzymatic reaction provides at least 20% of the total reaction volume of the enzymatic reaction, optionally wherein the prepared biological sample that is subjected to the enzymatic reaction provides up to 60% of the total reaction volume of the enzymatic reaction.

55. The method according to claim 42, wherein the method is for preparing a biological sample for amplification based detection of at least one pathogenic RNA target nucleic acid present in the biological sample without prior nucleic acid purification, the method comprising:
contacting the biological sample with an extraction solution comprising
(a) at least one non-ionic surfactant,
(b) at least one proteinaceous RNase inhibitor, and
(c) at least one reducing agent,
to prepare an admixture,
optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid;
subjecting at least an aliquot or all of the prepared biological sample to a reverse transcription and amplification reaction and performing the reaction.

56. The method according to claim 42, wherein the method is for preparing a biological sample for amplification based detection of at least one pathogenic RNA target nucleic acid without prior nucleic acid purification, the method comprising:
contacting the biological sample with an extraction solution comprising
(a) at least one non-ionic surfactant,
(b) at least one proteinaceous RNase inhibitor, and
(c) at least one reducing agent,
to prepare an admixture, wherein the admixture comprises
(i) the non-ionic surfactant originating from the extraction solution in a concentration that lies in a range of 0.1% to 10% (w/v), and
(ii) the reducing agent originating from the extraction solution in a concentration that lies in a range of 0.1 mM to 15 mM;
optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid;
subjecting at least an aliquot or all of the prepared biological sample to a reverse transcription and amplification reaction and performing the reaction.

57. The method according to claim 42, wherein the method is for preparing a biological sample for amplification based detection of at least one pathogenic RNA target nucleic acid without prior nucleic acid purification, the method comprising:
contacting the biological sample contained in medium with an extraction solution comprising:
(a) at least one non-ionic surfactant,
(b) at least one proteinaceous RNase inhibitor, and
(c) at least one reducing agent,
to prepare an admixture, wherein the admixture comprises
(i) the non-ionic surfactant originating from the extraction solution in a concentration that lies in a range of 0.1% to 10% (w/v), and
(ii) the reducing agent originating from the extraction solution in a concentration that lies in a range of 0.1 mM to 15 mM;
optionally wherein the method comprises heating the biological sample in the absence of the extraction solution to inactivate pathogens potentially comprised in the biological sample prior to contacting at least an aliquot of the pathogen heat-inactivated biological sample with the extraction solution;
incubating the admixture for at least 1 minute to provide the prepared biological sample for amplification based detection of the at least one pathogenic RNA target nucleic acid; and
subjecting at least an aliquot or all of the prepared biological sample to a reverse transcription and amplification reaction and performing the reaction;
wherein the prepared biological sample that is subjected to the reverse transcription and amplification reaction provides at least 30% of the total reaction volume of the reverse transcription and amplification reaction, and
wherein at least the steps of contacting the biological sample with the extraction solution to prepare the admixture,
incubating the admixture, and
performing the reverse-transcription amplification reaction, are performed within the same reaction vessel.

58. The method according to claim 42, wherein the method is for preparing a respiratory biological sample for amplification based detection of at least one pathogenic RNA target nucleic acid comprised in the biological sample without prior nucleic acid purification, the method comprising:
contacting the respiratory biological sample contained in medium with an extraction solution comprising
(a) at least one non-ionic surfactant,
(b) at least one proteinaceous RNase inhibitor, and
(c) at least one reducing agent,
to prepare an admixture,
optionally wherein the method comprises heating the respiratory biological sample contained in medium in the absence of the extraction solution to inactivate pathogens potentially comprised in the biological sample prior to contacting at least an aliquot of the pathogen heat-inactivated biological sample with the extraction solution;
incubating the admixture to provide the prepared biological sample for amplification based detection of the at least one pathogenic RNA target nucleic acid;
subjecting at least an aliquot or all of the prepared biological sample to a reverse transcription and amplification reaction and performing the reaction for detecting the presence or absence of the at least one pathogenic RNA target nucleic acid;
wherein the prepared biological sample that is subjected to the reverse transcription and amplification reaction provides at least 30% of the total reaction volume of the reverse transcription and amplification reaction, and
wherein at least the steps of
contacting the respiratory biological sample contained in medium with the extraction solution to prepare the admixture,
incubating the admixture, and
performing the reverse-transcription amplification reaction,
are performed within the same reaction vessel, and
wherein the reverse transcription and amplification reaction comprises primers suitable for amplifying one or more target nucleic acids derived from a severe acute respiratory syndrome-related coronavirus.

59. The method according to claim 42, wherein the method comprises
contacting the biological sample with an extraction composition comprising
(a) at least one non-ionic surfactant, wherein the admixture comprising the biological sample in contact with the extraction composition comprises the surfactant in a concentration that lies in a range of 0.3% to 5% (w/v),
(b) at least one proteinaceous RNase inhibitor, and
(c) at least one reducing agent, and
incubating the admixture comprising the biological sample in contact with the extraction composition to provide the prepared biological sample for amplification based detection of the target nucleic acid.

60. The method of claim 59, wherein the method for preparing the biological sample for amplification based detection of the target nucleic acid does not involve heating the biological sample in contact with the extraction composition to a temperature that would denature the comprised proteinaceous RNase inhibitor during incubation for providing the prepared biological sample.

61. The method of claim 59, wherein the method does not involve heating the biological sample in contact with the extraction composition to a temperature ≥ 75° C. for at least 2 minutes prior to subjecting at least an aliquot or all of the prepared biological sample to an enzymatic reaction selected from reverse transcription and amplification.

62. The method according to claim 42, wherein the method is for preparing a biological sample for amplification based detection of at least one RNA target nucleic acid present in the biological sample without prior nucleic acid purification, the method comprising
contacting the biological sample with an extraction solution comprising
(a) at least one non-ionic surfactant,
(b) at least one proteinaceous RNase inhibitor, and
(c) at least one reducing agent, to prepare an admixture,
wherein the method comprises heating the biological sample in the absence of the extraction solution to inactivate pathogens potentially comprised in the biological sample prior to contacting at least an aliquot of the pathogen heat-inactivated biological sample with the extraction solution, wherein the biological sample is heated to a temperature ≥ 85° C.;
incubating the admixture to provide the prepared biological sample for amplification based detection of the at least one RNA target nucleic acid.

63. The method according to claim 42, wherein the method is for preparing a biological sample for amplification based detection of at least one RNA target nucleic acid present in the biological sample without prior nucleic acid purification, the method comprising
contacting the biological sample with an extraction solution comprising
(a) at least one non-ionic surfactant,
(b) at least one proteinaceous RNase inhibitor, and
(c) at least one reducing agent, to prepare an admixture,
wherein the method comprises heating the biological sample in the absence of the extraction solution to a temperature ≥ 85° C. prior to contacting at least an aliquot of the heat-treated biological sample with the extraction solution,
optionally wherein heating occurs in the presence of a digestion solution that has been contacted with the biological sample, wherein the digestion solution comprises a proteolytic enzyme and a reducing agent, and
incubating the admixture to provide the prepared biological sample for amplification based detection of the at least one RNA target nucleic acid.

64. The method according to claim 42, wherein after incubation in contact with the extraction composition, the method comprises performing a reverse transcription amplification reaction to detect the presence or absence of a RNA target nucleic acid in the prepared biological sample.

65. The method according to claim 64, further comprising contacting the prepared biological sample with one or more components necessary for performing the reverse transcription amplification.

66. The method according to claim 64, wherein the one or more components necessary for performing the reverse transcription amplification are premixed with or added at the same time as the extraction composition.

67. A method for detecting the presence or absence of a pathogen in a biological sample based on amplifying at least one target nucleic acid derived from the pathogen, comprising
(A) preparing the biological sample for amplification based detection of the target nucleic acid, wherein preparing comprises
contacting the biological sample with an extraction composition comprising
(a) at least one surfactant,
(b) at least one nuclease inhibitor, and
(c) optionally at least one reducing agent, and
incubating the admixture comprising the biological sample in contact with the extraction composition to provide the prepared biological sample; and
(B) subjecting at least an aliquot or all of the prepared biological sample to an amplification reaction and amplifying the at least one target nucleic acid, optionally wherein a reverse transcription reaction is performed in order to reverse transcribe RNA to cDNA prior to amplification.

68. The method according to claim 67, wherein
(aa) the prepared biological sample that is subjected to the amplification reaction provides at least 20% of the total reaction volume of the amplification reaction, optionally wherein the prepared biological sample that is subjected to the amplification reaction provides up to 60% of the total reaction volume of the amplification reaction; and/or
(bb) wherein at least the steps of
contacting the biological sample with the extraction solution to prepare the admixture,
incubating the admixture, and
performing the reverse-transcription amplification reaction, are performed within the same reaction vessel.

69. The method according to claim 67, wherein the biological sample is a respiratory biological sample contained in a medium and the method is for amplification based detection of at least one pathogenic RNA target nucleic acid comprised in the biological sample without prior nucleic acid purification,
wherein the extraction composition used in (A) is an extraction solution comprising (a) a non-ionic surfactant, (b) a proteinaceous RNase inhibitor, and (c) a reducing agent, optionally wherein the extraction solution consists essentially of or consists of the aforementioned active ingredients comprised in liquid,
wherein in (B) a reverse transcription and amplification reaction is performed for detecting the presence or absence of the at least one pathogenic RNA target nucleic acid,
wherein the prepared biological sample that is subjected to the reverse transcription and amplification reaction provides at least 25% of the total reaction volume of the reverse transcription and amplification reaction,
wherein at least the steps of
contacting the respiratory biological sample contained in a medium with the extraction solution to prepare the admixture,
incubating the admixture comprising the extraction solution, the biological sample, and the medium to provide the prepared biological sample, and
performing the reverse-transcription amplification reaction, are performed within the same reaction vessel, and
wherein the reverse transcription and amplification reaction comprises primers suitable for amplifying one or more target nucleic acids derived from a severe acute respiratory syndrome-related coronavirus.

70. The method according to claim 67, wherein steps (A) and (B) are completed in 2 hours or less.

71. The method according to claim 67, wherein for performing the amplification reaction in (B) the prepared biological sample is in contact with the components used for performing the amplification or reverse transcription amplification reaction thereby providing an amplification reaction admixture and wherein the prepared amplification reaction admixture comprises:
(a) the prepared biological sample;
(b) a DNA polymerase;
(c) optionally a reverse transcriptase, which is included in case a reverse transcription amplification is performed;
(d) an amplification reaction buffer comprising a Mg²⁺ source, a buffering agent and optionally further additives;
(e) nucleotides, optionally wherein the nucleotides comprise modified nucleotides or dUTP; and
(f) primers for amplifying the one or more target nucleic acids and optionally probes.

72. The method according to claim 71, wherein the prepared biological sample is in contact with an amplification master mix comprising components (b) to (e) and optionally separately provided primers for amplifying the one or more target nucleic acids; and optionally probes.

73. The method according to claim 71, wherein the ionic strength of the amplification reaction buffer (d) or the amplification master mix comprising components (b) to (e) is reduced to thereby compensate the introduction of ions, in particular ions derived from alkali metal salts and/or chlorides, into the amplification reaction admixture due to the prepared biological sample.

74. The method according to claim 71, wherein the amplification reaction buffer (d) has one or more of the following characteristics:
(aa) the amplification reaction buffer (d) does not comprise sodium chloride in a concentration ≥ 30 mM;
(bb) the amplification reaction buffer (d) does not comprise potassium chloride in a concentration ≥ 30 mM;
(cc) the amplification reaction buffer (d) does not comprise potassium chloride or sodium chloride;
(dd) the alkali metal chloride concentration in the amplification reaction buffer (d) is ≤ 30 mM;
(ee) the alkali metal salt concentration in the amplification reaction buffer (d) is ≤ 30 mM.

75. The method according to claim 71, wherein the amplification reaction buffer (d):
(i) does not comprise potassium chloride or sodium chloride;
(ii) comprises a buffering agent that does not comprise chloride ions, optionally wherein the buffering agent is selected from tris(hydroxymethyl)aminomethane and 3-(N-morpholino)-propanesulphonic acid, and
(iii) is pH-adjusted with an organic acid.

76. The method according to claim 72, wherein the amplification master mix comprising components (b) to (e) has one or more of the following characteristics:
(aa) it does not comprise sodium chloride in a concentration ≥ 50 mM;
(bb) it does not comprise potassium chloride in a concentration ≥ 100 mM;
(cc) the alkali metal chloride concentration in the amplification master mix is ≤ 100 mM;
(dd) the alkali metal salt concentration in the amplification master mix is ≤100 mM;
(ff) the chloride ion concentration is ≤ 250 mM; and
optionally, wherein said amplification master mix is provided in concentrated form as 3x amplification master mix.

77. The method according to claim 72, wherein the amplification master mix comprising components (b) to (e) have one or both of the following characteristics:
(aa) it comprises a buffering agent that does not comprise chloride ions,
optionally wherein the buffering agent is selected from the group consisting of tris(hydroxymethyl)aminomethane, N-(tri(hydroxymethyl)methyl)glycine, N,N-bis(2-hydroxyethyl)glycine, 3-(N-morpholino)propanesulphonic acid, N-(2-hydroxyethyl)piperazine-N′-(2-ethanesulphonic acid), piperazine-1,4-bis(2-ethanesulphonic acid), N cyclohexyl-2-aminoethanesulphonic acid and 2-(N-morpholino)ethanesulphonic acid; and
(bb) the pH is adjusted with an organic acid.

78. A kit for obtaining a prepared biological sample for amplification based detection of at least one target nucleic acid present in the biological sample without prior target nucleic acid purification, the kit comprising
(a) an extraction composition comprising at least one surfactant, at least one nuclease inhibitor, and optionally at least one reducing agent; and
one or more of the following components:
(b) a DNA polymerase;
(c) a reverse transcriptase;
(d) an amplification reaction buffer comprising a Mg²⁺ source, a buffering agent and optionally further additives;
(e) nucleotides; and
(f) primers for amplifying the at least one target nucleic acid,
optionally, wherein components (b) to (e) or (b) to (f) are comprised in a single composition.

79. The kit according to claim 78, wherein the kit comprises an amplification master mix comprising components (b) to (e) or (b) to (f).

80. The kit according to claim 78, wherein the ionic strength of the amplification reaction buffer (d) or the amplification master mix is reduced to thereby compensate the introduction of ions, in particular ions derived from alkali metal salts and/or chlorides, into the amplification reaction admixture due to the prepared biological sample.

81. The kit according to claim 78, further comprising a digestion solution comprising a proteolytic enzyme and a reducing agent, optionally wherein the proteolytic enzyme is a protease and the reducing agent is selected from Tris(carboxyethyl)phosphine (TCEP), Dithiothreitol (DTT) and beta-mercaptoethanol.
